(19) United States
(12) Patent Application Publication
(10) Pub. No.: US 2008/0039437 A1
(43) Pub. Date: Feb. 14, 2008

(54) New Pyridine Analogues III

(75) Inventors: Thomas Antonsson, Molndal (SE); Peter Bach, Molndal (SE); David Brown, Molndal (SE); Ruth Bylund, Molndal (SE); Fabrizio Giordanetto, Molndal (SE); Lotta Jakobsson, Molndal (SE); Johan Johansson, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/772,269

(22) Filed: Jul. 2, 2007

(30) Foreign Application Priority Data
Jul. 4, 2006 (SE) 0601463-3
Jan. 12, 2007 (SE) 0700059-9
Oct. 4, 2006 (SE) 0602091-1

(51) Int. Cl. A61K 31/4545; A61K 31/4439; C07D 413/02; C07D 403/02

(52) U.S. Cl. 514/210.2; 514/340; 546/271.4; 514/316; 546/194; 546/268.1

(57) ABSTRACT

The present invention relates to certain new pyridin analogues of Formula (I) 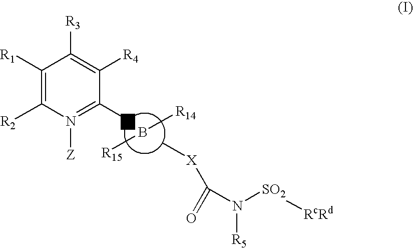
to processes for preparing such compounds, to their utility as P2Y₁₂ inhibitors and as anti-thrombotic agents etc, their use as medicaments in cardiovascular diseases as well as pharmaceutical compositions containing them.

### CROSS-REFERENCE TO RELATED APPLICATIONS

This application claims priority to Swedish Application No. 0700059-9 filed Jan. 12, 2007, to Swedish Application No. 0602091-1 filed Oct. 4, 2006, and to Swedish Application No. 0601463-3 filed Jul. 4, 2006, each which is incorporated herein by reference in its entirety.

### FIELD OF THE INVENTION

The present invention provides novel pyridine compounds, their use as medicaments, compositions containing them and processes for their preparation.

### BACKGROUND OF THE INVENTION

Platelet adhesion and aggregation are initiating events in arterial thrombosis. Although the process of platelet adhesion to the sub-endothelial surface may have an important role to play in the repair of damaged vessel walls, the platelet aggregation that this initiates can precipitate acute thrombotic occlusion of vital vascular beds, leading to events with high morbidity such as myocardial infarction and unstable angina. The success of interventions used to prevent or alleviate these conditions, such as thrombolysis and angioplasty is also compromised by platelet mediated occlusion or re-occlusion.

Haemostasis is controlled via a tight balance between platelet aggregation, coagulation and fibrinolysis. Thrombus formation under pathological conditions, like e.g. arteriosclerotic plaque rupture, is firstly initiated by platelet adhesion, activation and aggregation. This results not only in the formation of a platelet plug but also in the exposure of negatively charged phospholipids on the outer platelet membrane promoting blood coagulation. Inhibition of the build-up of the initial platelet plug would be expected to reduce thrombus formation and reduce the number of cardiovascular events as was demonstrated by the anti-thrombotic effect of e.g. Aspirin (BMJ 1994; 308: 81-106 Antiplatelet Trialists' Collaboration. Collaborative overview of randomised trials of antiplatelet therapy, I: Prevention of death, myocardial infarction, and stroke by prolonged antiplatelet therapy in various categories of patients).

Platelet activation/aggregation can be induced by a variety of different agonists. However, distinct intracellular signalling pathways have to be activated to obtain full platelet aggregation, mediated via G-proteins G_{q}, G_{12/13} and Gᵢ (Platelets, A D Michelson ed., Elsevier Science 2002, ISBN 0-12-493951-1; 197-213: D Woulfe, et al. Signal transduction during the initiation, extension, and perpetuation of platelet plug formation) In platelets, the G-protein coupled receptor P2Y₁₂ (previously also known as the platelet P_{2T}, P2T_{ac}, or P2Y_{cyc} receptor) signals via Gi, resulting in a lowering of intra-cellular cAMP and full aggregation (Nature 2001; 409: 202-207 G Hollopeter, et al. Identification of the platelet ADP receptor targeted by antithrombotic drugs.). Released ADP from dense-granules will positively feedback on the P2Y12 receptor to allow full aggregation.

Clinical evidence for the key-role of the ADP-P2Y₁₂ feedback mechanism is provided by the clinical use of clopidogrel, an thienopyridine prodrug which active metabolite selectively and irreversibly binds to the P2Y₁₂ receptor, that has shown in several clinical trials to be effective in reducing the risk for cardiovascular events in patients at risk (Lancet 1996; 348: 1329-39: CAPRIE Steering committee, A randomised, blinded, trial of clopidogrel versus aspirin in patients at risk of ischaemic events (CAPRIE); N Engl J Med 2001; 345 (7): 494-502): The Clopidogrel in Unstable Angina to prevent Recurrent Events Trial Investigators. Effects of clopidogrel in addition to aspirin in patients with acute coronary syndromes without ST-segment elevation.). In these studies, the clinical benefit of Clopidogrel treatment is associated with an increased rate of clinical bleeding. Published data suggest that reversible P2Y₁₂ antagonists could offer the possibility for high clinical benefit with a reduced bleeding risk as compared to thienopyridines (Sem Thromb Haemostas 2005; 31 (2): 195-204, van Giezen & R G Humphries. Preclinical and clinical studies with selective reversible direct P2Y₁₂ antagonists.

Accordingly it is an object of the present invention to provide potent, reversible and selective P2Y₁₂-antagonists as anti-thrombotic agents.

### SUMMARY OF THE INVENTION

We have now surprisingly found that certain pyridine compounds of Formula (I) or a pharmaceutically acceptable salt thereof are reversible and selective P2Y₁₂ antagonists, hereinafter referred to as the compounds of the invention. The compounds of the invention unexpectedly exhibit beneficial properties that render them particularly suitable for use in the treatment of diseases/conditions as described below. Examples of such beneficial properties are high potency, high selectivity, and an advantageous therapeutic window.

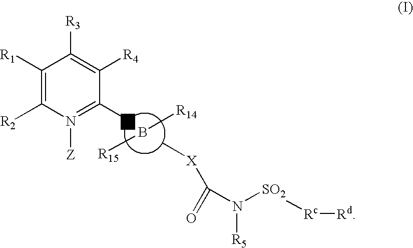

### DESCRIPTION OF EMBODIMENTS

According to the present invention there is provided a novel compound of formula (I) or a pharmaceutically acceptable salt thereof: 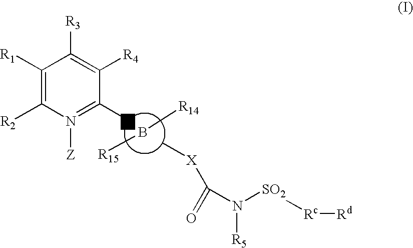
wherein

R₁ represents R₆OC(O), R₇C(O), R₁₆SC(O), R₁₇S, R₁₈C(S) or a group gII

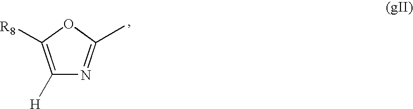

preferably R₁ represents R₆OC(O);

R₂ represents (C₁-C₁₂)alkyl optionally interrupted by oxygen and wherein the alkyl is substituted by one or more halogen (F, Cl, Br, I) atoms; further R₂ represents (C₁-C₁₂)alkoxy substituted by one or more halogen (F, Cl, Br, I) atoms;

R₃ represents H, CN, NO₂, halogen (F, Cl, Br, I), (C₁-C₁₂)alkyl optionally interrupted by oxygen and/or optionally substituted by OH, aryl, cycloalkyl, heterocyclyl or one or more halogen (F, Cl, Br, I) atoms; further R₃ represents (C₁-C₁₂)alkoxy optionally substituted by one or more halogen (F, Cl, Br, I) atoms; further R₃ represents (C₃-C₆)cycloalkyl, hydroxy(C₁-C₁₂)alkyl, (C₁-C₁₂)alkylC(O), (C₁-C₁₂)alkylthiOC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(3)}R^{b(3)} in which R^{a(3)} and R^{b(3)} independently represent H, (C₁-C₁₂)alkyl, (C₁-C₁₂)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₁₂)alkyl optionally interrupted by oxygen and/or optionally substituted by OH, COOH, (C₁-C₆)alkoxycarbonyl, aryl, cycloalkyl, heterocyclyl or one or more halogen (F, Cl, Br, I) atoms; further R₄ represents (C₃-C₆)cycloalkyl, hydroxy(C₁-C₁₂)alkyl, (C₁-C₁₂)alkylC(O), (C₁-C₁₂)alkylcycloalkyl, (C₁-C₁₂)alkoxy wherein the alkoxy group may optionally be substituted by one or more halogen (F, Cl, Br, I) atoms, OH and/or COOH and/or (C₁-C₆)alkoxycarbonyl; further R₄ represents (C₁-C₁₂)alkylthiOC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(4)}R^{b(4)} in which R^{a(4)} and R^{b(4)} independently represent H, (C₁-C₁₂)alkyl, (C₁-C₁₂)alkylC(O) or R^{a(4)} and R^{b(4)} together with the nitrogen atom represent piperidine, pyrrolidine, azetidine or aziridine;

Z represents O or is absent;

R₅ represents H or (C₁-C₁₂)alkyl;

R₆ represents (C₁-C₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₁₂)alkyl, aryl or heterocyclyl;

R₇ represents (C₁-C₁₂)alkyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₁₂)alkyl, aryl or heterocyclyl;

R₈ represents H, (C₁-C₁₂)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;

R₁₄ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl; further R₁₄ represents aryl, heterocyclyl, one or more halogen (F, Cl, Br, I) atoms, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl, a group of formula NR^{a(14)}R^{b(14)} in which R^{a(14)} and R^{b(14)} independently represent H, (C₁-C₁₂)alkyl, (C₁-C₁₂)alkylC(O), (C₁-C₁₂)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl; further R₁₅ represents aryl, heterocyclyl, one or more halogen (F, Cl, Br, I) atoms, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(15)}R^{b(15)} in which R^{a(15)} and R^{b(15)} independently represent H, (C₁-C₁₂)alkyl, (C₁-C₁₂)alkylC(O), (C₁-C₁₂)alkoxyC(O) or R^{a(15)} and R^{b(15)} together with the nitrogen atom represent piperidine, pyrrolidine, azetidine or aziridine;

R₁₆ represents (C₁-C₁₂)alkyl optionally interrupted by oxygen and/or optionally substituted by OH, aryl, cycloalkyl, heterocyclyl or one or more halogen (F, Cl, Br, I) atoms; further R₁₆ represents (C₃-C₆)cycloalkyl, hydroxy(C₂-C₁₂)alkyl, (C₁-C₁₂)alkoxy, (C₃-C₆)cycloalkoxy, aryl or heterocyclyl;

R₁₇ represents (C₁-C₁₂)alkyl optionally interrupted by oxygen and/or optionally substituted by OH, aryl, cycloalkyl, heterocyclyl or one or more halogen (F, Cl, Br, I) atoms; further R₁₇ represents (C₃-C₆)cycloalkyl, hydroxy(C₁-C₁₂)alkyl, (C₁-C₁₂)alkoxy, (C₃-C₆)cycloalkoxy, aryl or heterocyclyl;

R₁₈ represents (C₁-C₁₂)alkyl optionally interrupted by oxygen and/or optionally substituted by OH, aryl, cycloalkyl, heterocyclyl or one or more halogen (F, Cl, Br, I) atoms; further R₁₈ represents (C₃-C₆)cycloalkyl, hydroxy(C₁-C₁₂)alkyl, (C₁-C₁₂)alkoxy, (C₃-C₆)cycloalkoxy, aryl or heterocyclyl;

R^{c} is absent or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogen (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉, when present, represents H or (C₁-C₄)alkyl;

R^{d} represents (C₁-C₁₂)alkyl, (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, halogen substituted (C₁-C₁₂)alkoxy, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆)alkyl; further X may represent a group (—CH₂—)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) with the proviso that B is not piperazine, and further the B-ring/ring system is connected to X in another of its positions. The substituents R₁₄ and R₁₅ are connected to the B ring/ring system in such a way that no quarternary ammonium compounds are formed (by these connections).

Preferred values as well as embodiments of each variable group or combinations thereof are as follows. Such values or embodiments may be used where appropriate with any of the values, definitions, claims, aspects or embodiments defined hereinbefore or hereinafter. In particular, each may be used as an individual limitation on the broadest definition as well as any other of the embodiments of formula (I).

For the avoidance of doubt it is to be understood that where in this specification a group is qualified by “hereinbefore defined”, “defined hereinbefore” or “defined above” the said group encompasses the first occurring and broadest definition as well as each and all of the particular definitions for that group.

It will be understood that when formula I compounds contain a chiral centre, the compounds of the invention may exist in, and be isolated in, optically active or racemic form. The invention includes any optically active or racemic form of a compound of formula I which act as P2Y₁₂ receptor antagonists. The synthesis of optically active forms may be carried out by standard techniques of organic chemistry well known in the art, for example by, resolution of a racemic mixture, by chiral chromatography, synthesis from optically active starting materials or by asymmetric synthesis.

It will also be understood that the compounds of the formula I may exhibit the phenomenon of tautomerism, the present invention includes any tautomeric form of a compound of formula I which is a P2Y₁₂ receptor antagonist.

It will also be understood that in so far as compounds of the present invention exist as solvates, and in particular hydrates, these are included as part of the present invention.

It is also to be understood that generic terms such as “alkyl” include both the straight chain and branched chain groups such as butyl and tert-butyl. However, when a specific term such as “butyl” is used, it is specific for the straight chain or “normal” butyl group, branched chain isomers such as “t-butyl” being referred to specifically when intended.

In one embodiment alkyl is unsubstituted or substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “alkyl” includes both linear or branched chain groups, optionally substituted by one or more halogens (F, Cl, Br, I) or mixed halogen atoms.

One embodiment of alkyl when substituted by one or more halogen atoms (F, Cl, Br, I) is, for example, alkyl substituted by one or more fluorine atoms. Another embodiment of halogen substituted alkyl includes perfluoroalkyl groups such as trifluoromethyl.

The term “cycloalkyl” generally denotes a substituted or unsubstituted (C₃-C₆), unless other chain length specified, cyclic hydrocarbon.

In one embodiment cycloalkyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “alkoxy” includes both linear or branched chain groups, optionally substituted by one or more halogens (F, Cl, Br, I) or mixed halogen atoms.

The term aryl denotes a substituted or unsubstituted (C₆-C₁₄) aromatic hydrocarbon and includes, but is not limited to, phenyl, naphthyl, tetrahydronaphtyl, indenyl, indanyl, antracenyl, fenantrenyl, and fluorenyl.

In one embodiment aryl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “heterocyclyl” denotes a substituted or unsubstituted, 4- to 10-membered monocyclic or multicyclic ring system in which one or more of the atoms in the ring or rings is an element other than carbon, for example nitrogen, oxygen or sulfur, especially 4-, 5- or 6-membered aromatic or aliphatic hetorocyclic groups, and includes, but is not limited to azetidine, furan, thiophene, pyrrole, pyrroline, pyrrolidine, dioxolane, oxathiolane, oxazolane, oxazole, thiazole, imidazole, imidazoline, imidazolidine, pyrazole, pyrazoline, pyrazolidine, isothiazole, oxadiazole, furazan, triazole, thiadiazole, pyran, pyridine as well as pyridine-N-oxide, piperidine, dioxane, morpholine, dithiane, oxathiane, thiomorpholine, pyridazine, pyrimidine, pyrazine, piperazine, triazine, thiadiazine, dithiazine, azaindole, azaindoline, indole, indoline, naphthyridine, benzoxadiazole, dihydrobenzodioxin, benzothiophene, benzothiadiazole, imidazothiazole, 2,3-dihydrobenzofuran, isoxazole, 3-benzisoxazole, 1,2-benzisoxazole, dihydropyrazole groups, and shall be understood to include all isomers of the above identified groups. For the above groups, e.g. azetidinyl, the term “azetidinyl” as well as “azetidinylene”, etc., shall be understood to include all possible regio isomers. It is further to be understood that the term heterocyclyl may be embodified by one selection among the given possible embodiments for a variable and embodified by another (or the same) selection for another variable, eg. R₄ when selected as heterocyclyl may be a furan, when R^{d} (also when selected as heterocyclyl) may be a pyrrole.

In one embodiment heterocyclyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

In another embodiment of the invention the heterocyclyl group comprises an aromatic 5-membered or 6-membered heterocyclic ring containing one, two or three heteroatoms selected from nitrogen, oxygen and sulphur, and an aromatic 5-membered or 6-membered heterocyclic ring containing one, two or three heteroatoms selected from nitrogen, oxygen and sulphur which is fused to a benzene ring.

In an alternative embodiment of the invention the heterocyclyl group is a non-aromatic 5-membered or 6-membered heterocyclic ring containing one, two or three heteroatoms selected from nitrogen, oxygen and sulphur, fused to a benzene ring.

In a further embodiment of the invention the heterocyclyl group is a group chosen among furyl, pyrrolyl, thienyl, pyridyl, N-oxido-pyridyl, pyrazinyl, pyrimidinyl, pyridazinyl, imidazolyl, oxazolyl, isooxazolyl, thiazolyl, isothiazolyl, oxadiazolyl, 1,2,3-triazolyl, 1,2,4-triazolyl, benzfuranyl, quinolyl, isoquinolyl, benzimidazolyl, indolyl, benzdihydrofuranyl, benzodioxolyl (such as 1,3-benzodioxolyl), benzoxadiazole, dihydrobenzodioxin, benzothiophene, benzothiadiazole, imidazothiazole, 2,3-dihydrobenzofuran, isoxazole, dihydropyrazole and benzdioxanyl (such as 1,4-benzdioxanyl). More particular values include, for example, furyl, pyrrolyl, thienyl, pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole, dihydropyrazole and benzdioxanyl (such as 1,4-benzdioxanyl).

In an even further embodiment of the invention the heterocyclyl group is a group chosen among furyl, pyrrolyl, thienyl, pyridyl, N-oxido-pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole or dihydropyrazole.

In one embodiment of the invention R₁ represents R₆OC(O).

In a further embodiment of the invention R₁ is R₆OC(O) wherein R₆ can be methyl, ethyl, 2-hydroxyethyl, 2,2,2-trifluoroethyl, isopropyl, cyclo-propyl, isobutyl, n-butyl, cyclobutyl, n-propyl, tertbutyl, cyclo-pentyl, 2,2-dimethylpropyl, benzyl and 4-fluorobenzyl.

R₁ may also be embodified by the group gII, 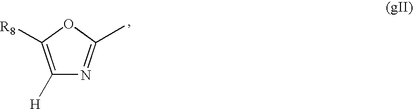
in which R₈ is selected from H, (C₁-C₆)alkyl, such as methyl or ethyl.

In another embodiment for the group R₈ this group can be chosen among hydrogen, methyl, ethyl, n-propyl and n-butyl.

Embodiments for R₂ include, for example, (C₁-C₄)alkyl substituted by one or more halogen (F, Cl, Br, I) atoms or mixed halogen atoms.

In another embodiment R₂ is (C₁-C₄)alkyl substituted with one or more fluoro atoms.

Another further embodiment for R₂ is (C₁-C₄)alkyl substituted with one or more fluoro atoms and optionally one or more chlorine atom.

In a further embodiment R₂ is (C₁-C₄)alkyl substituted with one or more fluoro atoms and one or more chlorine atom.

In an even further embodiment R₂ is methyl substituted with one or more fluoro atoms.

An alternative further embodiment for R₂ is methyl substituted with two fluoro atoms.

Another embodiment for R₂ is (C₁-C₄)alkoxy substituted with one or more fluoro atoms and optionally one or more chlorine atom.

A specific embodiment for R₂ is ethoxy substituted with one or more fluoro atoms.

Embodiments for R₃ include, for example, H, methyl, methylsulfinyl, hydroxymethyl, methoxy or amino unsubstituted or optionally substituted with one or two methyl groups.

Other embodiments for R₃ include H or amino unsubstituted or optionally substituted with one or two methyl groups.

Embodiments for R₄ include H, halogen such as chloro, methyl, cyano, nitro, amino unsubstituted or optionally substituted with one or two methyl groups and further includes 4-methoxy-4-oxobutoxy, 3-carboxy-propoxy and methylcarbonyl.

In one embodiment of the invention Z is absent.

In another embodiment of the invention Z represents O.

In one embodiment of the invention R₅ represents hydrogen or methyl. In another embodiment of the invention R₅ is hydrogen.

Further embodiments for R₈ include, hydrogen, methyl and ethyl.

Further embodiments for R₁₄ include, for example, hydrogen, methyl, amino, tert-butyloxycarbonyl, tert-butyloxycarbonyl-imino, 2-carboxyethyl and 3-tert-butoxy-3-oxo-propyl.

Other further embodiments for R₁₄ include, for example, hydrogen, methyl, tert-butyloxycarbonyl-imino, and amino.

In one embodiment of the invention R₁₅ represents H.

Embodiments for R^{d} includes alkyl, cycloalkyl, aryl or heterocyclyl, more particularly, aryl or aromatic heterocyclyl.

In one embodiment of the invention R^{d} is (C₁-C₆)alkyl, (C₃-C₆)cycloalkyl optionally substituted with alkyl, aryl or one or more halogen (F, Cl, Br, I) atoms or mixed halogen atoms.

Another embodiment for R^{d} include aryl such as phenyl and aromatic heterocyclyl such as thienyl.

Other embodiments of R^{d} include phenyl which optionally may be substituted.

In a special embodiment R^{d} represents aryl, heterocyclyl or (C₃-C₆)cycloalkyl, and anyone of these groups are optionally substituted with one or more halogen (F, Cl, Br, I) atoms or mixed halogen atoms, and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, halogen substituted (C₁-C₁₂)alkoxy, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

Even further embodiments for R^{d} include phenyl optionally substituted at the 2, 3, 4 or 5-positions as well as any combination thereof. Example of substituents are cyano, tetrazol-5-yl, methoxy, trifluoromethoxy, methyl, trifluoromethyl, fluoro, chloro, bromo, methylsulfonyl, nitro, 3-methyl-5-oxo-4,5-dihydro-1H-pyrazol-1-yl. Two adjacent positions (e.g. 2,3) may also be connected to form a ring. Example of such a substituent is 2-naphtyl. Further more specific values for heteroaryls are 2-chloro-5-thienyl, 3-bromo-5-chloro-2-thienyl, 2,1,3-benzoxadiazol-4-yl, 2,4-dimethyl-1,3-thiazol-5-yl, 2,3-dihydro-1,4-benzodioxin-6-yl, 5-chloro-3-methyl-1-benzothien-2-yl, 2,1,3-benzothiadiazol-4-yl, 2,5-dimethyl-3-furyl, 6-chloroimidazo[2,1-b][1,3]thiazol-5-yl, 2,3-dihydro-1-benzofuran-5-yl, 5-chloro-3-thienyl, 5-isoxazol-5-yl-2-thienyl, 5-isoxazol-3-yl-2-thienyl, 4-bromo-5-chloro-2-thienyl, 5-bromo-6-chloropyridin-3-yl, 5-bromo-2-thienyl, 5-pyridin-2-yl-2-thienyl, 2,5-dichloro-3-thienyl, 4,5-dichloro-2-thienyl, benzothien-3-yl, 2,5-dimethyl-3-thienyl, 3-thienyl, 2-thienyl, 5-methylisoxazol-4-yl, pyridin-3-yl, [1-methyl-5-(trifluoromethyl)-1H-pyrazol-3-yl]-2-thienyl, 5-chloro-1,3-dimethyl-1H-pyrazol-4-yl, 4-[(4-chlorophenyl)sulfonyl]-3-methyl-2-thienyl, 5-(methoxycarbonyl)-2-furyl and 4-(methoxycarbonyl)-5-methyl-2-furyl.

In one embodiment of the invention R^{c} represents an unsubstituted or monosubstituted or disubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogen (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e R^{c}R^{d} represents an aryl-(C₁-C₄)alkylene group with any substituents according to above.

In a preferred embodiment of the invention R^{c} represents an unsubstituted or monosubstituted or disubstituted (C₁-C₃)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogen (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e R^{c}R^{d} represents an aryl-(C₁-C₃)alkylene group with any substituents according to above.

In a further embodiment of the invention R^{c} is absent or represents an unsubstituted or monosubstituted or disubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogen (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl. In a further preferred embodiment of the invention R^{c} is absent or represents an unsubstituted or monosubstituted or disubstituted (C₁-C₃)alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogen (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl.

In a particular embodiment of the invention R^{c} is absent or represents a C₁-alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogen (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl.

In one embodiment of the invention R^{c} is absent.

In one embodiment of the invention R₁₉, when present, represents hydrogen.

In another embodiment of the invention R₁₉, when present, represents methyl.

In a most particular embodiment of the invention R^{c}R^{d} represents a benzyl group, or a benzyl group which is substituted according to what is described in connection to substitution of the aryl group.

In one embodiment of the invention X represents a single bond.

In another embodiment of the invention X represents single bond or methylene (—CH₂—). In yet another embodiment X represents imino (—NH—). In a further embodiment X represents methylene (—CH₂—).

Suitable values for the B ring/ring system include, for example, diazepanylene, piperidinylene, pyrrolidinylene and azetidinylene, wherein anyone of them may be presents in any of their isomeric forms (e.g. piperazin-tetrahydropyridazine-tetrahydropyrimidin).

A further embodiment of the B ring/ring system is when B is selected from the group consisting of piperidinylene and azetidinylene.

An alternative embodiment of the B ring/ring system is when B is piperidinylene.

Another alternative embodiment of the B ring/ring system is when B is azetidinylene.

Embodiments for the B ring/ring system include, for example, diazepanylene, piperidinylene, pyrrolidinylene and azetidinylene. Further embodiments include these groups which are substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₁₂)alkyl optionally substituted by one or more of halogen (F, Cl, Br, I) or mixed halogen atoms, OH, aryl, cycloalkyl and heterocyclyl.

In an alternative to the embodiment for the B ring/ring system above, the embodiments include piperidinylene, pyrrolidinylene or azetidinylene groups which optionally are substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₆)alkyl optionally substituted by one or more of halogen (F, Cl, Br, I) or mixed halogen atoms, OH, aryl, cycloalkyl and heterocyclyl.

A 2nd embodiment of formula I is defined by:

R₁ represents R₆OC(O), R₇C(O), R₁₆SC(O), R₁₇S, R₁₈C(S) or a group gII,

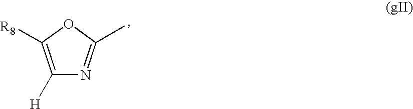

R₂ represents (C₁-C₆)alkyl optionally interrupted by oxygen and wherein the alkyl is substituted by one or more halogen (F, Cl, Br, I) atoms; further R₂ represents (C₁-C₆)alkoxy substituted by one or more halogen (F, Cl, Br, I) atoms;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen atoms; further R₃ represents (C₁-C₆)alkoxy optionally substituted by one or more halogen (F, Cl, Br, I) atoms; further R₃ represents (C₃-C₆)cycloalkyl, hydroxy(C₁-C₆)alkyl, (C₁-C₆)alkylC(O), (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₆)alkyl optionally interrupted by oxygen and/or optionally substituted by OH, COOH, (C₁-C₆)alkoxycarbonyl, aryl, cycloalkyl, heterocyclyl or one or more halogen atoms; further R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by one or more halogen (F, Cl, Br, I) atoms, OH and/or COOH and/or (C₁-C₃)alkoxycarbonyl; further R₄ represents (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

Z represents O or is absent;

R₅ represents H or (C₁-C₆)alkyl;

R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₆)alkyl, aryl or heterocyclyl;

R₇ represents (C₁-C₆)alkyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₆)alkyl, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents aryl, heterocyclyl, one or more halogen (F, Cl, Br, I) atoms, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₅ represents aryl, heterocyclyl, one or more halogen (F, Cl, Br, I) atoms, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;

R₁₆ represents (C₁-C₆)alkyl optionally interrupted by oxygen and/or optionally substituted by OH, aryl, cycloalkyl, heterocyclyl or one or more halogen (F, Cl, Br, I) atoms; further R₁₆ represents (C₃-C₆)cycloalkyl, hydroxy(C₂-C₆)alkyl, (C₁-C₆)alkoxy, (C₃-C₆)cycloalkoxy, aryl, or heterocyclyl;

R₁₇ represents (C₁-C₆)alkyl optionally interrupted by oxygen and/or optionally substituted by OH, aryl, cycloalkyl, heterocyclyl or one or more halogen (F, Cl, Br, I) atoms; further R₁₇ represents (C₃-C₆)cycloalkyl, hydroxy(C₁-C₆)alkyl, (C₁-C₆)alkoxy, (C₃-C₆)cycloalkoxy, aryl or heterocyclyl;

R₁₈ represents (C₁-C₆)alkyl optionally interrupted by oxygen and/or optionally substituted by OH, aryl, cycloalkyl, heterocyclyl or one or more halogen (F, Cl, Br, I) atoms; further R₁₈ represents (C₃-C₆)cycloalkyl, hydroxy(C₁-C₆)alkyl, (C₁-C₆)alkoxy, (C₃-C₆)cycloalkoxy, aryl or heterocyclyl;

R^{c} is absent or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogen (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉, when present, represents H or (C₁-C₄)alkyl;

R^{d} represents (C₁-C₆)alkyl, (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, halogen substituted (C₁-C₆)alkoxy, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆)alkyl; further X may represent a group (—CH₂—)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) with the proviso that B is not piperazine, and further the B-ring/ring system is connected to X in another of its positions. The substituents R₁₄ and R₁₅ are connected to the B ring/ring system in such a way that no quarternary ammonium compounds are formed (by these connections).

A 3rd embodiment of formula I is defined by:

R₁ represents R₆OC(O), R₁₆SC(O), or a group gII,

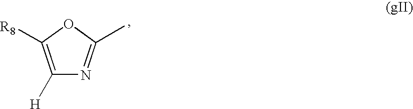

R₂ represents (C₁-C₆)alkyl optionally interrupted by oxygen and wherein the alkyl is substituted by one or more halogen (F, Cl, Br, I) atoms; further R₂ represents (C₁-C₆)alkoxy substituted by one or more halogen (F, Cl, Br, I) atoms;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen atoms; further R₃ represents (C₁-C₆)alkoxy optionally substituted by one or more halogen (F, Cl, Br, I) atoms; further R₃ represents (C₃-C₆)cycloalkyl, hydroxy(C₁-C₆)alkyl, (C₁-C₆)alkylC(O), (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₆)alkyl optionally interrupted by oxygen and/or optionally substituted by OH, COOH, aryl, cycloalkyl, heterocyclyl or one or more halogen atoms; further R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by one or more halogen (F, Cl, Br, I) atoms, OH and/or COOH and/or methoxycarbonyl; further R₄ represents (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

Z represents O or is absent;

R₅ represents H or (C₁-C₆)alkyl;

R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₆)alkyl, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₆)alkyl, (C₁-C₆)alkoxy, (C₃-C₆)cycloalkoxy, aryl or heterocyclyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents aryl, heterocyclyl, one or more halogen (F, Cl, Br, I) atoms, (C₃-C₆)cycloalkyl, hydroxy(C₁-C₆)alkyl, (C₁-C₆)alkoxy, (C₃-C₆)cycloalkoxy, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₅ represents aryl, heterocyclyl, one or more halogen (F, Cl, Br, I) atoms, (C₃-C₆)cycloalkyl, hydroxy(C₁-C₆)alkyl, (C₁-C₆)alkoxy, (C₃-C₆)cycloalkoxy, or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;

R₁₆ is ethyl;

R^{c} is absent or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogen (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉, when present, represents H or (C₁-C₄)alkyl;

R^{d} represents (C₁-C₆)alkyl, (C₃-C₈)cycloalkyl, aryl or heterocyclyl, and anyone of these groups optionally substituted with one or more halogen (F, Cl, Br, I) atoms and/or one or more of the following groups, CN, NO₂, (C₁-C₆)alkyl, (C₁-C₆)alkoxy, halogen substituted (C₁-C₆)alkyl, halogen substituted (C₁-C₆)alkoxy, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆)alkyl; further X may represent a group (—CH₂—)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) with the proviso that B is not piperazine, and further the B-ring/ring system is connected to X in another of its positions. The substituents R₁₄ and R₁₅ are connected to the B ring/ring system in such a way that no quarternary ammonium compounds are formed (by these connections).

A 4th embodiment of formula I is defined by:

R₁ represents R₆OC(O);

R₂ represents (C₁-C₄)alkyl substituted by one or more halogen (F, Cl, Br, I) atoms;

R₃ represents H;

R₄ represents CN or halogen (F, Cl, Br, I);

Z is absent;

R₅ represents H;

R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms;

R₁₄ represents H;

R₁₅ represents H;

R^{c} is absent or represents an unsubstituted (C₁-C₄)alkylene group;

R^{d} represents (C₁-C₆)alkyl, (C₃-C₈)cycloalkyl, aryl or heterocyclyl, and anyone of these groups optionally substituted with one or more halogen (F, Cl, Br, I) atoms and/or one or more of the following groups, CN, (C₁-C₆)alkyl, (C₁-C₆)alkoxy, halogen substituted (C₁-C₆)alkyl, halogen substituted (C₁-C₆)alkoxy;

X represents a single bond or methylene (—CH₂—); and

B is a monocyclic, 4 to 7-membered heterocyclic ring/ring system comprising one or more nitrogen and optionally one or more atoms selected from oxygen or sulphur, which nitrogen is connected to the pyridine-ring (according to formula I) with the proviso that B is not piperazine, and further the B-ring/ring system is connected to X in another of its positions. The substituents R₁₄ and R₁₅ are connected to the B ring/ring system in such a way that no quarternary ammonium compounds are formed (by these connections).

A 5th embodiment of formula I is defined by that:

R₁ is ethoxycarbonyl or isopropoxycarbonyl;

R₂ is chosen from a group consisting of fluoromethyl, chloromethyl, difluoromethyl, trifluoromethyl, pentafluoroethyl, 1-fluoroethyl, 2-fluoroethoxy, 2,2,2,-trifluoroethoxy, difluoromethoxy and 2,2-difluoroethoxy;

R₃ is H;

R₄ is chosen from chloro or cyano;

Z is absent;

R₅ is H;

R₆ is ethyl or isopropyl;

R₁₄ is H;

R₁₅ is H;

R^{c} is absent or is chosen from methylene (—CH₂—) or ethylene (—CH₂CH₂—);

R^{d} is chosen from a group consisting of n-butyl, 4-methylcyclohexyl, phenyl, 3-methylphenyl, 4-methylphenyl, 2-(trifluoromethoxy)phenyl, 4-(trifluoromethoxy)phenyl, 2-fluorophenyl, 3-fluorophenyl, 4-fluorophenyl, 2-chlorophenyl, 3-chlorophenyl, 4-chlorophenyl, 2,4-dichlorophenyl, 2-cyanophenyl, 3-cyanophenyl, 4-cyanophenyl, 3-methoxyphenyl, 2-naphtyl, 2,6-difluorophenyl, 4-fluoro-3-methylphenyl, 2-chloro-4-fluorophenyl, 2,3,6-trifluorophenyl, 2,4-difluorophenyl, 4-chloro-2-fluorophenyl, 5-fluoro-2-methylphenyl, 2-fluoro-5-methylphenyl, 3-methoxyphenyl, 3,4-difluorophenyl, 4-hydroxymethylphenyl and 5-chloro-2-thienyl;

X represents a single bond or methylene (—CH₂—);

B is chosen from the group consisting of 4-piperidin-1-ylene, 3-pyrrolidine-1-ylene and 3-azetidin-1-ylene, and the substituents R₁₄ and R₁₅ are connected to the B ring/ring system, in such a way that no quarternary ammonium compounds are formed (by these connections).

In a 6th embodiment of formula (I), formula (I) is defined as being any compound(s) of formula (Ia)-(Id):

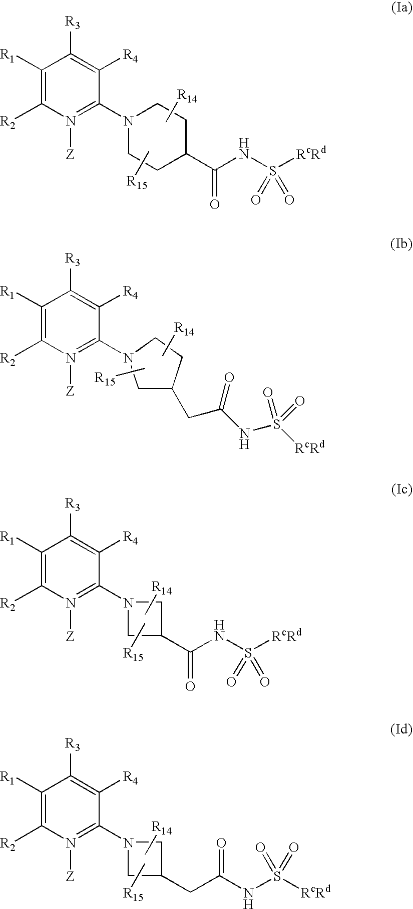

In the above Ia to Id the various values of Z and R (except R₅ being H) are as defined above and include the previously mentioned embodiments.

In a 7^{th} embodiment formula (I) is defined as being any compound(s) of formula (Iaa)-(Idd):

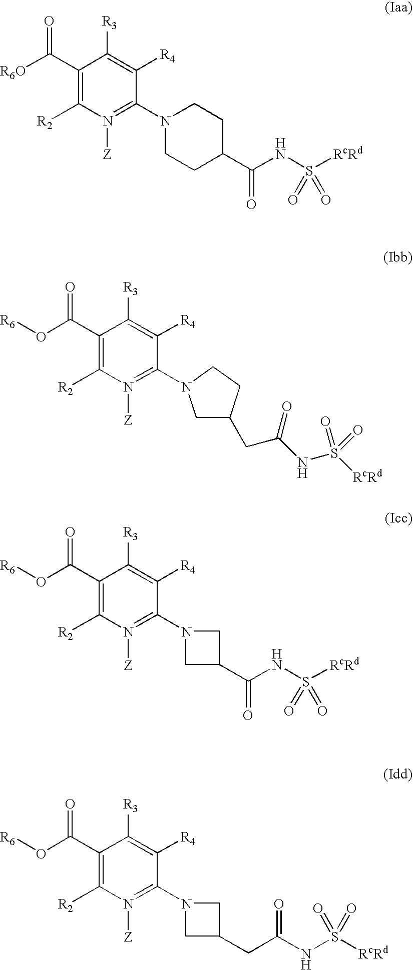

In the above Iaa to Idd the various values of Z and R (except R₅, R₁₄ and R₁₅, all being H) are as defined above and include the previously mentioned embodiments.

Examples of specific compounds according to the invention can be selected from:
ethyl 6-(4-{[(benzylsulfonyl)amino]carbonyl}piperidin-1-yl)-5-chloro-2-(difluoromethyl)nicotinate;
ethyl 6-(4-{[(benzylsulfonyl)amino]carbonyl}piperidin-1-yl)-5-cyano-2-(difluoromethyl)nicotinate;
ethyl 6-(4-{[(benzylsulfonyl)amino]carbonyl}piperidin-1-yl)-5-cyano-2-(trifluoromethyl)nicotinate;
ethyl 6-(3-{[(benzylsulfonyl)amino]carbonyl}azetidin-1-yl)-5-cyano-2-(difluoromethyl)nicotinate;
ethyl 6-(3-{[(benzylsulfonyl)amino]carbonyl}azetidin-1-yl)-5-cyano-2-(trifluoromethyl)nicotinate;
ethyl 6-(4-{[(benzylsulfonyl)amino]carbonyl}piperidin-1-yl)-5-cyano-2-(fluoromethyl)nicotinate;
ethyl 6-(3-{[(benzylsulfonyl)amino]carbonyl}azetidin-1-yl)-5-cyano-2-(fluoromethyl)nicotinate;
ethyl 5-cyano-2-(difluoromethyl)-6-{4-[({[(4-methylcyclohexyl)methyl]sulfonyl}amino)carbonyl]piperidin-1-yl}nicotinate;
ethyl 5-cyano-2-(difluoromethyl)-6-[3-({[(2-fluorobenzyl)sulfonyl]amino}carbonyl)azetidin-1-yl]nicotinate;
ethyl 5-cyano-2-(difluoromethyl)-6-[4-({[(2-fluorobenzyl)sulfonyl]amino}carbonyl)piperidin-1-yl]nicotinate;
ethyl 5-cyano-2-(difluoromethyl)-6-[4-({[(3-fluorobenzyl)sulfonyl]amino}carbonyl)piperidin-1-yl]nicotinate;
ethyl 5-cyano-2-(difluoromethyl)-6-[4-({[(4-fluorobenzyl)sulfonyl]amino}carbonyl)piperidin-1-yl]nicotinate;
ethyl 6-[4-({[(2-chlorobenzyl)sulfonyl]amino}carbonyl)piperidin-1-yl]-5-cyano-2-(difluoromethyl)nicotinate;
ethyl 6-[4-({[(3-chlorobenzyl)sulfonyl]amino}carbonyl)piperidin-1-yl]-5-cyano-2-(difluoromethyl)nicotinate;
ethyl 6-[4-({[(4-chlorobenzyl)sulfonyl]amino}carbonyl)piperidin-1-yl]-5-cyano-2-(difluoromethyl)nicotinate;
ethyl 5-cyano-2-(difluoromethyl)-6-[4-({[(3-methylbenzyl)sulfonyl]amino}carbonyl)piperidin-1-yl]nicotinate;
ethyl 5-cyano-2-(difluoromethyl)-6-[4-({[(4-methylbenzyl)sulfonyl]amino}carbonyl)piperidin-1-yl]nicotinate;
ethyl 5-cyano-6-[4-({[(2,4-dichlorobenzyl)sulfonyl]amino}carbonyl)piperidin-1-yl]-2-(difluoromethyl)nicotinate;
ethyl 5-cyano-2-(difluoromethyl)-6-[3-({[(3-fluorobenzyl)sulfonyl]amino}carbonyl)azetidin-1-yl]nicotinate;
ethyl 5-cyano-2-(difluoromethyl)-6-[3-({[(4-fluorobenzyl)sulfonyl]amino}carbonyl)azetidin-1-yl]nicotinate;
ethyl 6-[3-({[(2-chlorobenzyl)sulfonyl]amino}carbonyl)azetidin-1-yl]-5-cyano-2-(difluoromethyl)nicotinate;
ethyl 6-[3-({[(3-chlorobenzyl)sulfonyl]amino}carbonyl)azetidin-1-yl]-5-cyano-2-(difluoromethyl)nicotinate;
ethyl 6-[3-({[(4-chlorobenzyl)sulfonyl]amino}carbonyl)azetidin-1-yl]-5-cyano-2-(difluoromethyl)nicotinate;
ethyl 5-cyano-2-(difluoromethyl)-6-[3-({[(3-methylbenzyl)sulfonyl]amino}carbonyl)azetidin-1-yl]nicotinate;
ethyl 5-cyano-2-(difluoromethyl)-6-[3-({[(4-methylbenzyl)sulfonyl]amino}carbonyl)azetidin-1-yl]nicotinate;
ethyl 5-cyano-6-[3-({[(2,4-dichlorobenzyl)sulfonyl]amino}carbonyl)azetidin-1-yl]-2-(difluoromethyl)nicotinate;
ethyl 5-cyano-2-(difluoromethyl)-6-{3-[({[(4-methylcyclohexyl)methyl]sulfonyl}amino)carbonyl]azetidin-1-yl}nicotinate;
ethyl 5-cyano-6-[3-({[(3-cyanophenyl)sulfonyl]amino}carbonyl)azetidin-1-yl]-2-(difluoromethyl)nicotinate;
ethyl 5-cyano-6-[3-({[(4-cyanophenyl)sulfonyl]amino}carbonyl)azetidin-1-yl]-2-(difluoromethyl)nicotinate;
ethyl 5-cyano-2-(difluoromethyl)-6-{3-[({[4-(trifluoromethoxy)phenyl]sulfonyl}amino)carbonyl]azetidin-1-yl}nicotinate;
ethyl 5-cyano-2-(difluoromethyl)-6-{3-[({[2-(trifluoromethoxy)phenyl]sulfonyl}amino)carbonyl]azetidin-1-yl}nicotinate;
ethyl 5-cyano-6-[3-({[(2-cyanobenzyl)sulfonyl]amino}carbonyl)azetidin-1-yl]-2-(difluoromethyl)nicotinate;
ethyl 5-cyano-2-(difluoromethyl)-6-(3-{[(2-naphthylsulfonyl)amino]carbonyl}azetidin-1-yl)nicotinate;
ethyl 6-(3-{[(butylsulfonyl)amino]carbonyl}azetidin-1-yl)-5-cyano-2-(difluoromethyl)nicotinate;
ethyl 5-cyano-6-[4-({[(3-cyanophenyl)sulfonyl]amino}carbonyl)piperidin-1-yl]-2-(difluoromethyl)nicotinate;
ethyl 5-cyano-6-[4-({[(4-cyanophenyl)sulfonyl]amino}carbonyl)piperidin-1-yl]-2-(difluoromethyl)nicotinate;
ethyl 5-cyano-2-(difluoromethyl)-6-{4-[({[4-(trifluoromethoxy)phenyl]sulfonyl}amino)carbonyl]piperidin-1-yl}nicotinate;
ethyl 5-cyano-2-(difluoromethyl)-6-{4-[({[2-(trifluoromethoxy)phenyl]sulfonyl}amino)carbonyl]piperidin-1-yl}nicotinate;
ethyl 5-cyano-6-[4-({[(2-cyanobenzyl)sulfonyl]amino}carbonyl)piperidin-1-yl]-2-(difluoromethyl)nicotinate;
ethyl 5-cyano-2-(difluoromethyl)-6-(4-{[(2-naphthylsulfonyl)amino]carbonyl}piperidin-1-yl)nicotinate;
ethyl 6-(4-{[(butylsulfonyl)amino]carbonyl}piperidin-1-yl)-5-cyano-2-(difluoromethyl)nicotinate;
ethyl 6-(3-{2-[(benzylsulfonyl)amino]-2-oxoethyl}pyrrolidin-1-yl)-5-cyano-2-(trifluoromethyl)nicotinate;
ethyl 5-cyano-6-[3-(2-oxo-2-{[(2-phenylethyl)sulfonyl]amino}ethyl)pyrrolidin-1-yl]-2-(trifluoromethyl)nicotinate;
ethyl 6-[3-(2-{[(5-chloro-2-thienyl)sulfonyl]amino}-2-oxoethyl)pyrrolidin-1-yl]-5-cyano-2-(trifluoromethyl)nicotinate;
ethyl 5-cyano-6-[3-({[(4-fluorobenzyl)sulfonyl]amino}carbonyl)azetidin-1-yl]-2-(trifluoromethyl)nicotinate;
ethyl 5-cyano-6-[3-({[(3-fluorobenzyl)sulfonyl]amino}carbonyl)azetidin-1-yl]-2-(trifluoromethyl)nicotinate;
ethyl 5-cyano-6-[3-({[(2-fluorobenzyl)sulfonyl]amino}carbonyl)azetidin-1-yl]-2-(trifluoromethyl)nicotinate;
ethyl 5-cyano-6-[3-({[(4-methylbenzyl)sulfonyl]amino}carbonyl)azetidin-1-yl]-2-(trifluoromethyl)nicotinate;
ethyl 5-cyano-6-[3-({[(3-methylbenzyl)sulfonyl]amino}carbonyl)azetidin-1-yl]-2-(trifluoromethyl)nicotinate;
ethyl 6-[3-({[(4-chlorobenzyl)sulfonyl]amino}carbonyl)azetidin-1-yl]-5-cyano-2-(trifluoromethyl)nicotinate;
ethyl 6-[3-({[(2-chlorobenzyl)sulfonyl]amino}carbonyl)azetidin-1-yl]-5-cyano-2-(trifluoromethyl)nicotinate;
ethyl 6-[3-({[(3-chlorobenzyl)sulfonyl]amino}carbonyl)azetidin-1-yl]-5-cyano-2-(trifluoromethyl)nicotinate;
ethyl 5-cyano-6-[3-({[(2,4-dichlorobenzyl)sulfonyl]amino}carbonyl)azetidin-1-yl]-2-(trifluoromethyl)nicotinate;
ethyl 6-[3-({[(5-chloro-2-thienyl)sulfonyl]amino}carbonyl)azetidin-1-yl]-5-cyano-2-(trifluoromethyl)nicotinate;
ethyl 5-cyano-6-[4-({[(4-fluorobenzyl)sulfonyl]amino}carbonyl)piperidin-1-yl]-2-(trifluoromethyl)nicotinate;
ethyl 5-cyano-6-[4-({[(3-fluorobenzyl)sulfonyl]amino}carbonyl)piperidin-1-yl]-2-(trifluoromethyl)nicotinate;
ethyl 5-cyano-6-[4-({[(2-fluorobenzyl)sulfonyl]amino}carbonyl)piperidin-1-yl]-2-(trifluoromethyl)nicotinate;
ethyl 5-cyano-6-[4-({[(4-methylbenzyl)sulfonyl]amino}carbonyl)piperidin-1-yl]-2-(trifluoromethyl)nicotinate;
ethyl 5-cyano-6-[4-({[(3-methylbenzyl)sulfonyl]amino}carbonyl)piperidin-1-yl]-2-(trifluoromethyl)nicotinate;
ethyl 6-[4-({[(4-chlorobenzyl)sulfonyl]amino}carbonyl)piperidin-1-yl]-5-cyano-2-(trifluoromethyl)nicotinate;
ethyl 6-[4-({[(2-chlorobenzyl)sulfonyl]amino}carbonyl)piperidin-1-yl]-5-cyano-2-(trifluoromethyl)nicotinate;
ethyl 6-[4-({[(3-chlorobenzyl)sulfonyl]amino}carbonyl)piperidin-1-yl]-5-cyano-2-(trifluoromethyl)nicotinate;
ethyl 5-cyano-6-[4-({[(2,4-dichlorobenzyl)sulfonyl]amino}carbonyl)piperidin-1-yl]-2-(trifluoromethyl)nicotinate;
ethyl 6-[4-({[(5-chloro-2-thienyl)sulfonyl]amino}carbonyl)piperidin-1-yl]-5-cyano-2-(trifluoromethyl)nicotinate;
ethyl 5-cyano-6-[3-({[(2-fluorobenzyl)sulfonyl]amino}carbonyl)azetidin-1-yl]-2-(fluoromethyl)nicotinate;
ethyl 5-cyano-6-[3-({[(3-fluorobenzyl)sulfonyl]amino}carbonyl)azetidin-1-yl]-2-(fluoromethyl)nicotinate;
ethyl 5-cyano-6-[3-({[(4-fluorobenzyl)sulfonyl]amino}carbonyl)azetidin-1-yl]-2-(fluoromethyl)nicotinate;
ethyl 6-[3-({[(2-chlorobenzyl)sulfonyl]amino}carbonyl)azetidin-1-yl]-5-cyano-2-(fluoromethyl)nicotinate;
ethyl 6-[3-({[(3-chlorobenzyl)sulfonyl]amino}carbonyl)azetidin-1-yl]-5-cyano-2-(fluoromethyl)nicotinate;
ethyl 6-[3-({[(4-chlorobenzyl)sulfonyl]amino}carbonyl)azetidin-1-yl]-5-cyano-2-(fluoromethyl)nicotinate;
ethyl 5-cyano-2-(fluoromethyl)-6-[3-({[(3-methylbenzyl)sulfonyl]amino}carbonyl)azetidin-1-yl]nicotinate;
ethyl 5-cyano-2-(fluoromethyl)-6-[3-({[(4-methylbenzyl)sulfonyl]amino}carbonyl)azetidin-1-yl]nicotinate;
ethyl 5-cyano-6-[3-({[(2,4-dichlorobenzyl)sulfonyl]amino}carbonyl)azetidin-1-yl]-2-(fluoromethyl)nicotinate;
ethyl 5-cyano-2-(fluoromethyl)-6-{3-[({[(4-methylcyclohexyl)methyl]sulfonyl}amino)carbonyl]azetidin-1-yl}nicotinate;
ethyl 5-cyano-6-[4-({[(2-fluorobenzyl)sulfonyl]amino}carbonyl)piperidin-1-yl]-2-(fluoromethyl)nicotinate;
ethyl 5-cyano-6-[4-({[(3-fluorobenzyl)sulfonyl]amino}carbonyl)piperidin-1-yl]-2-(fluoromethyl)nicotinate;
ethyl 5-cyano-6-[4-({[(4-fluorobenzyl)sulfonyl]amino}carbonyl)piperidin-1-yl]-2-(fluoromethyl)nicotinate;
ethyl 6-[4-({[(2-chlorobenzyl)sulfonyl]amino}carbonyl)piperidin-1-yl]-5-cyano-2-(fluoromethyl)nicotinate;
ethyl 6-[4-({[(3-chlorobenzyl)sulfonyl]amino}carbonyl)piperidin-1-yl]-5-cyano-2-(fluoromethyl)nicotinate;
ethyl 6-[4-({[(4-chlorobenzyl)sulfonyl]amino}carbonyl)piperidin-1-yl]-5-cyano-2-(fluoromethyl)nicotinate;
ethyl 5-cyano-2-(fluoromethyl)-6-[4-({[(3-methylbenzyl)sulfonyl]amino}carbonyl)piperidin-1-yl]nicotinate;
ethyl 5-cyano-2-(fluoromethyl)-6-[4-({[(4-methylbenzyl)sulfonyl]amino}carbonyl)piperidin-1-yl]nicotinate;
ethyl 5-cyano-6-[4-({[(2,4-dichlorobenzyl)sulfonyl]amino}carbonyl)piperidin-1-yl]-2-(fluoromethyl)nicotinate;
ethyl 5-cyano-2-(fluoromethyl)-6-{4-[({[(4-methylcyclohexyl)methyl]sulfonyl}amino)carbonyl]piperidin-1-yl}nicotinate;
ethyl 6-(3-{2-[(benzylsulfonyl)amino]-2-oxoethyl}azetidin-1-yl)-5-cyano-2-(difluoromethyl)nicotinate;
ethyl 5-cyano-6-(3-{[(2-cyanobenzyl)sulfonyl]carbamoyl}azetidin-1-yl)-2-(trifluoromethyl)nicotinate;
ethyl 5-cyano-6-(3-{[(2,6-difluorobenzyl)sulfonyl]carbamoyl}azetidin-1-yl)-2-(fluoromethyl)nicotinate;
ethyl 5-cyano-2-(fluoromethyl)-6-(3-{[(4-fluoro-3-methylbenzyl)sulfonyl]carbamoyl}azetidin-1-yl)nicotinate;
ethyl 6-(3-{[(2-chloro-4-fluorobenzyl)sulfonyl]carbamoyl}azetidin-1-yl)-5-cyano-2-(fluoromethyl)nicotinate;
ethyl 5-cyano-2-(fluoromethyl)-6-(3-{[(2,3,6-trifluorobenzyl)sulfonyl]carbamoyl}azetidin-1-yl)nicotinate;
ethyl 5-cyano-6-(3-{[(2,4-difluorobenzyl)sulfonyl]carbamoyl}azetidin-1-yl)-2-(fluoromethyl)nicotinate;
ethyl 6-(3-{[(4-chloro-2-fluorobenzyl)sulfonyl]carbamoyl}azetidin-1-yl)-5-cyano-2-(fluoromethyl)nicotinate;
ethyl 5-cyano-6-(3-{[(2,6-difluorobenzyl)sulfonyl]carbamoyl}azetidin-1-yl)-2-(difluoromethyl)nicotinate;
ethyl 5-cyano-2-(difluoromethyl)-6-(3-{[(4-fluoro-3-methylbenzyl)sulfonyl]carbamoyl}azetidin-1-yl)nicotinate;
ethyl 6-(3-{[(2-chloro-4-fluorobenzyl)sulfonyl]carbamoyl}azetidin-1-yl)-5-cyano-2-(difluoromethyl)nicotinate;
ethyl 5-cyano-2-(difluoromethyl)-6-(3-{[(5-fluoro-2-methylbenzyl)sulfonyl]carbamoyl}azetidin-1-yl)nicotinate;
ethyl 5-cyano-6-(3-{[(2,4-difluorobenzyl)sulfonyl]carbamoyl}azetidin-1-yl)-2-(difluoromethyl)nicotinate;
ethyl 6-(3-{[(4-chloro-2-fluorobenzyl)sulfonyl]carbamoyl}azetidin-1-yl)-5-cyano-2-(difluoromethyl)nicotinate;
ethyl 5-cyano-6-(3-{[(2,6-difluorobenzyl)sulfonyl]carbamoyl}azetidin-1-yl)-2-(trifluoromethyl)nicotinate;
ethyl 5-cyano-6-(3-{[(4-fluoro-3-methylbenzyl)sulfonyl]carbamoyl}azetidin-1-yl)-2-(trifluoromethyl)nicotinate;
ethyl 6-(3-{[(2-chloro-4-fluorobenzyl)sulfonyl]carbamoyl}azetidin-1-yl)-5-cyano-2-(trifluoromethyl)nicotinate;
ethyl 5-cyano-6-(3-{[(5-fluoro-2-methylbenzyl)sulfonyl]carbamoyl}azetidin-1-yl)-2-(trifluoromethyl)nicotinate;
ethyl 5-cyano-6-(3-{[(2,3,6-trifluorobenzyl)sulfonyl]carbamoyl}azetidin-1-yl)-2-(trifluoromethyl)nicotinate;
ethyl 6-(3-{[(4-chloro-2-fluorobenzyl)sulfonyl]carbamoyl}azetidin-1-yl)-5-cyano-2-(trifluoromethyl)nicotinate;
ethyl 5-cyano-6-(4-{[(2,6-difluorobenzyl)sulfonyl]carbamoyl}piperidin-1-yl)-2-(difluoromethyl)nicotinate;
ethyl 5-cyano-2-(difluoromethyl)-6-(4-{[(4-fluoro-3-methylbenzyl)sulfonyl]carbamoyl}piperidin-1-yl)nicotinate;
ethyl 5-cyano-2-(fluoromethyl)-6-(3-{[(2-fluoro-5-methylbenzyl)sulfonyl]carbamoyl}azetidin-1-yl)nicotinate;
ethyl 5-cyano-6-(4-{[(2-fluoro-5-methylbenzyl)sulfonyl]carbamoyl}piperidin-1-yl)-2-(trifluoromethyl)nicotinate;
ethyl 5-cyano-6-(3-{[(2-fluoro-5-methylbenzyl)sulfonyl]carbamoyl}azetidin-1-yl)-2-(trifluoromethyl)nicotinate;
ethyl 5-cyano-2-(difluoromethyl)-6-(4-{[(2-fluoro-5-methylbenzyl)sulfonyl]carbamoyl}piperidin-1-yl)nicotinate;
ethyl 5-cyano-2-(difluoromethyl)-6-(3-{[(3-methoxybenzyl)sulfonyl]carbamoyl}azetidin-1-yl)nicotinate;
ethyl 6-{4-[(benzylsulfonyl)carbamoyl]piperidin-1-yl}-5-cyano-2-(pentafluoroethyl)nicotinate;
ethyl 6-{3-[(benzylsulfonyl)carbamoyl]azetidin-1-yl}-5-cyano-2-(pentafluoroethyl)nicotinate;
ethyl 6-{3-[(benzylsulfonyl)carbamoyl]azetidin-1-yl}-5-cyano-2-(1-fluoroethyl)nicotinate;
ethyl 6-{4-[(benzylsulfonyl)carbamoyl]piperidin-1-yl}-5-cyano-2-(1-fluoroethyl)nicotinate;
ethyl 6-(4-{[(2-chloro-4-fluorobenzyl)sulfonyl]carbamoyl}piperidin-1-yl)-5-cyano-2-(fluoromethyl)nicotinate;
ethyl 5-cyano-6-(4-{[(2,4-difluorobenzyl)sulfonyl]carbamoyl}piperidin-1-yl)-2-(fluoromethyl)nicotinate;
ethyl 6-{4-[(benzylsulfonyl)carbamoyl]piperidin-1-yl}-2-(chloromethyl)-5-cyanonicotinate;
ethyl 5-cyano-2-(difluoromethyl)-6-(3-{[(2-fluoro-5-methylbenzyl)sulfonyl]carbamoyl}azetidin-1-yl)nicotinate;
ethyl 6-{3-[(benzylsulfonyl)carbamoyl]azetidin-1-yl}-2-(chloromethyl)-5-cyanonicotinate;
ethyl 5-cyano-6-(3-{[(3,4-difluorobenzyl)sulfonyl]carbamoyl}azetidin-1-yl)-2-(difluoromethyl)nicotinate;
ethyl 5-cyano-6-(4-{[(3,4-difluorobenzyl)sulfonyl]carbamoyl}piperidin-1-yl)-2-(difluoromethyl)nicotinate;
ethyl 5-cyano-6-(4-{[(2,4-difluorobenzyl)sulfonyl]carbamoyl}piperidin-1-yl)-2-(difluoromethyl)nicotinate;
ethyl 6-{4-[(benzylsulfonyl)carbamoyl]piperidin-1-yl}-5-cyano-2-(2-fluoroethoxy)nicotinate;
ethyl 6-{3-[(benzylsulfonyl)carbamoyl]azetidin-1-yl}-5-cyano-2-[(2,2,2-trifluoroethoxy)methyl]nicotinate;
ethyl 6-{4-[(benzylsulfonyl)carbamoyl]piperidin-1-yl}-5-cyano-2-[(2,2,2-trifluoroethoxy)methyl]nicotinate;
ethyl 6-{4-[(benzylsulfonyl)carbamoyl]piperidin-1-yl}-5-cyano-2-(difluoromethoxy)nicotinate;
ethyl 6-{4-[(benzylsulfonyl)carbamoyl]piperidin-1-yl}-5-cyano-2-(2,2-difluoroethoxy)nicotinate;
ethyl 6-{4-[(benzylsulfonyl)carbamoyl]piperidin-1-yl}-5-cyano-2-(2,2,2-trifluoroethoxy)nicotinate;
ethyl 5-cyano-2-(difluoromethyl)-6-[3-({[4-(hydroxymethyl)benzyl]sulfonyl}carbamoyl)azetidin-1-yl]nicotinate;
ethyl 5-cyano-2-(difluoromethyl)-6-[4-({[4-(hydroxymethyl)benzyl]sulfonyl}carbamoyl)piperidin-1-yl]nicotinate;
ethyl 6-{3-[(benzylsulfonyl)carbamoyl]azetidin-1-yl}-5-cyano-2-(2,2-difluoroethoxy)nicotinate;
ethyl 5-cyano-2-(2,2-difluoroethoxy)-6-(3-{[(4-fluorobenzyl)sulfonyl]carbamoyl}azetidin-1-yl)nicotinate;
ethyl 5-cyano-2-(2,2-difluoroethoxy)-6-(3-{[(2-fluorobenzyl)sulfonyl]carbamoyl}azetidin-1-yl)nicotinate;
ethyl 5-cyano-6-(3-{[(2,4-difluorobenzyl)sulfonyl]carbamoyl}azetidin-1-yl)-2-(2,2-difluoroethoxy)nicotinate;

isopropyl 6-{3-[(benzylsulfonyl)carbamoyl]azetidin-1-yl}-5-cyano-2-(difluoromethyl)nicotinate;
ethyl 5-cyano-6-[3-({[(4-methylcyclohexyl)methyl]sulfonyl}carbamoyl)azetidin-1-yl]-2-(trifluoromethyl)nicotinate;
and pharmaceutically acceptable salts thereof.
   Processes

The following processes together with the intermediates are provided as a further feature of the present invention.

Compounds of formula (I) may be prepared by the following processes a1-a9;

a1) Compounds of formula (I) in which R₁, R₂, R₃, R₄, B, R₅, R₁₄, R₁₅, Z, R^{c} and R^{d} are defined as in formula (I) above, X is a single bond, a carbon or (—CH₂—), (n=2-6), can be formed by reacting a compound of formula (II), in which R₁, R₂, R₃, R₄, B, Z, R₁₄, and R₁₅ are defined 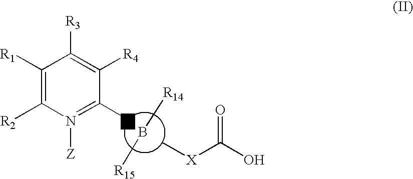
as in formula (I) above, X is a single bond, a carbon or (—CH₂—), (n=2-6), with a compound of formula (III) in which R₅, R^{c} and R^{d} are defined as in formula (I) above.
R₅—NHSO₂—R^{c}—R^{d} (III)

The reaction is generally carried out in an inert organic solvent such as dichloromethane at ambient temperature. The reaction may be carried out using standard conditions or in the presence of PyBrop, TBTU, EDCI or the combination of EDCI and HOBT. Optionally, the reaction may be carried out in the presence of an organic base such as triethylamine or DIPEA.

a2) Compounds of formula (I) in which R₁, R₂, R₃, R₄, B, R₅, R₁₄, R₁₅, Z, R^{c} and R^{d} are defined as in formula (I) above, X is a nitrogen, (—CH₂—NH—) or a single bond connected to a nitrogen which is a member of the B ring, can be formed by reacting a compound of formula (IV), in which R₁, R₂, R₃, R₄, B, R₁₄, and R₁₅ are defined as in formula (I) above and X is a nitrogen, (—CH₂—NH₂) or a hydrogen that is connected to a nitrogen which is a member of the B-ring, with a compound of the general 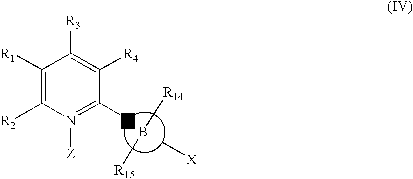
formula (III) which is defined as above.

The reaction is generally carried out in an inert solvent such as DCM. The reaction may be carried out in the presence of CDI. Optionally, the reaction may be carried out in the presence of an organic base such as triethylamine, DBU or DIPEA.

a3) Compounds of formula (I) in which R₁, R₂, R₃, R₄, B, R₁₄, R₁₅, Z, R^{c} and R^{d} are defined as in formula (I) above, R₅ is a hydrogen, X is a nitrogen, (—CH₂—NH—) or a single bond connected to a nitrogen which is a member of the B ring, can be formed by reacting a compound of formula (IV) which is defined in a2) above, with a compound of formula (V)
O═C═N—SO₂—R^{c}R^{d} (V)
in which R^{c} and R^{d} are defined as in formula (I) above.

The reaction is generally carried out in an inert solvent such as THF. Optionally, the reaction may be carried out in the presence of an organic base such as triethylamine or DIPEA.

a4) Compounds of formula (I) in which R₁, R₂, R₃, R₄, B, R₅, R₁₄, R₁₅, Z, R^{c} and R^{d} are defined as in formula (I) above, X is a nitrogen, (—CH₂—NH—) or a single bond connected to a nitrogen which is a member of the B ring, can be formed by reacting a compound of formula (IV) which is defined in above, with a compound of formula (VI),
R^{d}R^{c}—SO₂NR₅—COOCH₂CCl₃ (VI)
in which R₅, R^{c} and R^{d} are defined as in formula (I) above. The reaction is generally carried out in a solvent such as DMA. Optionally, the reaction may be carried out in the presence of an organic base such as triethylamine or DIPEA.

a5) Compounds of formula (I) may also be prepared by reacting a compound of formula (VII) in which R₁, R₂, R₃, R₄ and Z are defined as in formula (I) above and L is a suitable leaving group, such as chloro, bromo, iodo, fluoro, triflate (OTf) or tosylate (OTs), 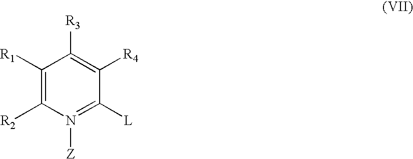
with a compound of the general formula (VIII) in which B, X, R₅, R₁₄, R₁₅, R^{c} and R^{d} are defined as in formula (I) above.

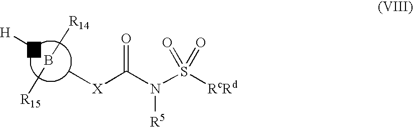

The reaction is generally carried out in an inert solvent such as DMA. Optionally, the reaction may be carried out in the presence of an organic base such as triethylamine or DIPEA.

The reaction is generally carried out at elevated temperatures using standard equipment or in a single-node microwave oven.

For some compounds, it is advantageous to carry out the reaction in ethanol in the presence of an organic base such as triethylamine or DIPEA.

a6) Compounds of formula (I) where R₁ represents R₆OC(O) and R₂, R₃, R₄, B, R₅, R₆, R₁₄, R₁₅, X, Z, R^{c} and R^{d} are defined as in formula (I) above, can be transesterified using standard procedures or by reacting with R_{6′}, —O⁻Li⁺ reagent, to become another compound of the general formula (I) wherein R₁ becomes R_{6′}OC(O).

a7) A compound of formula (I) in which R₁, R₂, R₃, R₄, B, R₅, R₁₄, R₁₅, Z and R^{d} are defined as in formula (I) above and R^{c} represents imino (—NH—) or (C₁-C₄)alkylimino in which the imino group could be substituted using standard conditions or using an alkylating agent like L-R₁₉, in which R₁₉ is defined as in formula (I) above and L is a leaving group exemplified by chloro, bromo, iodo, triflate (OTf) or tosylate (OTs), to give compounds of formula (I) in which R₁, R₂, R₃, R₄, B, R₅, R₁₄, R₁₅, Z and R^{d} are defined as in formula (I) above and R^{c} represents N-substituted imino (—NR₁₉—) or N-substituted (C₁-C₄)alkylimino (—N(R₁₉)—((C₁-C₄)alkyl), optionally in the presence of a strong base such as NaH.

a8) The compounds of formula (I) in which R₁, R₃, R₄, B, R₅, R₁₄, R₁₅, X, Z, R^{c} and R^{d} are as defined in formula (I) above, R₂ is (C₁-C₁₂)alkoxy defined as in formula (I) above can be prepared by reacting a compound of formula (IX) 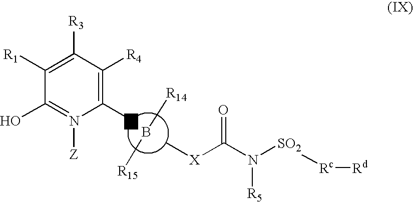
in which R₁, R₃, R₄, B, R₅, R₁₄, R₁₅, X, Z, R^{c} and R^{d} are as defined in formula (I) above with a compound of formula (X)
L-R_{2′} (X)
in which R2′ is (C₁-C₁₂)alkyl substituted by one or more halogen atoms and L is a leaving group such as chloro, bromo, iodo, triflate (OTf) or tosylate (OTs).

The reaction is carried out in an inert organic solvent such as DMA, THF or CH₃CN. The reaction may be carried out using standard conditions or in the presence of a suitable base such as sodium hydride, DIPEA, silver carbonate or potassium carbonate. The reaction may be carried out at ambient temperature or at elevated temperatures using standard equipment or a single node microwave oven.

a9) Compounds of formula (I) in which R₁, R₃, R₄, B, R₅, R₆, R₁₄, R₁₅, X, R^{c} and R^{d} are as defined in formula (I) above, R₂ is a substituted (C₁-C₁₂)alkoxy group defined as in formula (I) above can be prepared by reacting a compound of formula (IXA) 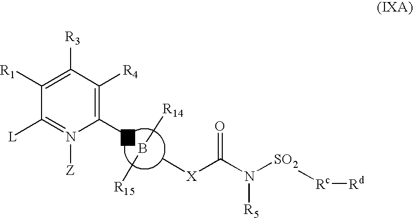
in which R₁, R₃, R₄, Z, B, R₅, R₆, R₁₄, R₁₅, X, R^{c} and R^{d} are as defined in formula (I) above and L is a suitable leaving group such as Cl, Br, I tosylate (OTs) or triflate (OTf) with the corresponding substituted (C₁-C₁₂)alcohol.

The reaction may be performed using standard conditions or in the presence of a palladium catalyst such as or Pd(PPh₃)₄ or Pd₂(dba)₃ in combination with a suitable phosphine ligand such as PPh₃ or XANTPHOS. The reaction may be carried out in an inert solvent such as DCM, THF or dioxane optionally in the presence of a base such as DIPEA. The reaction may be carried out at ambient temperature or at elevated temperatures using standard equipment or a single node microwave oven.

The intermediates referred to above may be prepared by, for example, the methods/processes outlined below.

b1) The compounds of formula (II) in which R₁, R₂, R₃, R₄, B, Z, R₁₄, and R₁₅ are defined as in formula (I) above, X is a single bond, a carbon or (—CH₂—)ₙ (n=2-6), may be prepared by reacting a compound of formula (VII) defined above with a compound of the general formula (XII), 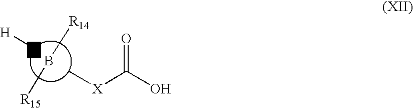
in which B, R₁₄, R₁₅ are defined as in formula (I) above and X is a single bond, a carbon or (—CH₂—)ₙ (n=2-6).

The reaction is generally carried out at elevated temperatures using standard equipment or in a single-node microwave oven. The reaction can be carried out in an inert solvent such as ethanol, DMA or a mixture of solvents such as ethanol-water. Optionally the reaction may be carried out in the presence of an organic base such as TEA or DIPEA.

b2) The compounds of formula (II) in which R₁, R₃, R₄, B, Z, R₁₄, and R₁₅ are defined as in formula (I) above, X is a single bond, a carbon or (—CH₂—)ₙ (n=2-6) and R₂ is (C₁-C₁₂)alkoxy defined as in formula (I) above may be prepared by reacting a compound of formula (IIB) in which R₁, R₃, R₄ B, ZR₁₄, and R₁₅ are defined as in formula (I) above, X is a single bond, a carbon or (—CH₂—)ₙ (n=2-6) 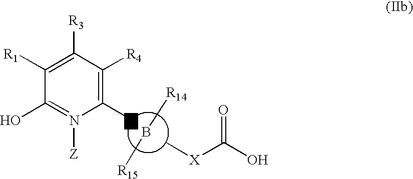
with a compound of formula (X) defined as above.

The reaction is carried out in an inert organic solvent such as DMA, THF or CH₃CN. The reaction may be carried out using standard conditions or in the presence of a suitable base such as sodium hydride, DIPEA, silver carbonate or potassium carbonate. The reaction may be carried out at ambient temperature or at elevated temperatures using standard equipment or a single node microwave oven.

b3) Compounds of formula (II) in which R₁, R₃, R₄, B, Z, R₁₄, and R₁₅ are defined as in formula (I) above, X is a single bond, a carbon or (—CH₂—)ₙ (n=2-6) and R₂ is (C₁-C₁₂)alkoxy defined as in formula (I) above may be prepared by reacting a compound of formula (IIA) 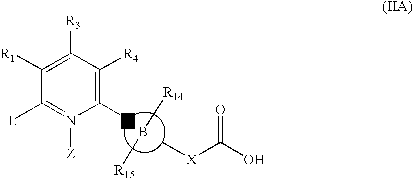
in which R₁, R₃, R₄, B, Z, R₁₄, and R₁₅ are defined as in formula (I) above, X is a single bond, a carbon or (—CH₂—)ₙ (n=2-6) and L is a suitable leaving group such as Cl, Br, I, tosylate (OTs) or triflate (OTf) with the corresponding substituted (C₁-C₁₂)alcohol. The reaction may be performed using standard conditions in the presence of a palladium catalyst such as or Pd(PPh₃)₄ or Pd₂(dba)₃ in combination with a suitable phosphine ligand such as PPh₃ or XANTPHOS.

The reaction may be carried out in an inert solvent such as DCM, THF or dioxane optionally in the presence of a base such as DIPEA.

The reaction may be carried out at ambient temperature or at elevated temperatures using standard equipment or a single node microwave oven.

c1) Compounds of formula (IV) which are defined as above may be prepared by reacting the corresponding compound of formula (VII) which is defined above, with a compound of formula (XIII) in which B, R₁₄, R₁₅ are defined as in formula (I) above, X is a nitrogen, (—CH₂—NH₂) or a hydrogen that is connected to a nitrogen which is a member of the B ring.

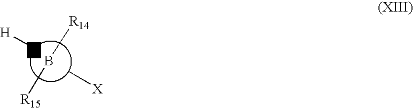

The reaction is generally carried out at elevated temperatures using standard equipment or in a single-node microwave oven. The reaction can be carried out in an inert solvent such as ethanol, DMA or a mixture of solvents such as ethanol-water. Optionally the reaction may be carried out in the presence of an organic base such as TEA or DIPEA.

c2) Compounds of general formula (IV) above wherein R₁, R₃, R₄, B, Z, R₁₄, R₁₅, are defined as in formula (I), and X is a nitrogen, (—CH₂—NH₂) or a hydrogen that is connected to a nitrogen which is a member of the B ring and R₂ is (C₁-C₁₂)alkoxy defined as in formula (I) above may be prepared by reacting a compound of formula (IVB) wherein R₁, R₃, R₄, B, Z, R₁₄, R₁₅, are defined as in formula (I) and X is a nitrogen, (—CH₂—NH₂) or a hydrogen that is connected to a nitrogen which is a member of the B ring 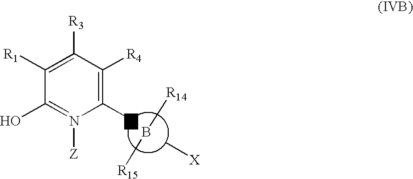
with a compound of formula (X) defined as above.

The reaction is carried out in an inert organic solvent such as DMA, THF or CH₃CN. The reaction may be carried out using standard conditions or in the presence of a suitable base such as sodium hydride, DIPEA, silver carbonate or potassium carbonate. The reaction may be carried out at ambient temperature or at elevated temperatures using standard equipment or a single node microwave oven.

c3) Compounds of general formula (IV) above wherein R₁, R₃, R₄, B, Z, R₁₄, R₁₅, are defined as in formula (I) and X is a nitrogen, (—CH₂—NH₂) or a hydrogen that is connected to a nitrogen which is a member of the B ring and R₂ is (C₁-C₁₂)alkoxy defined as in formula (I) above may be prepared by reacting a compound of formula (IVA) 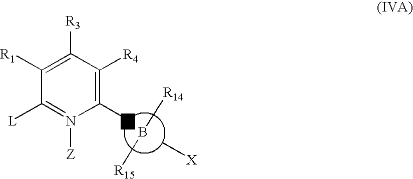
wherein R₁, R₃, R₄, B, Z, R₁₄, R₁₅, are defined as in formula (I) and X is a nitrogen, (—CH₂—NH₂) or a hydrogen that is connected to a nitrogen which is a member of the B ring and L is a suitable leaving group such as Cl, Br, I tosylate (OTs) or triflate (OTf) with the corresponding substituted (C₁-C₁₂)alcohol.

The reaction may be performed using standard conditions in the presence of a palladium catalyst such as or Pd(PPh₃)₄ or Pd₂(dba)₃ in combination with a suitable phosphine ligand such as PPh₃ or XANTPHOS.

The reaction may be carried out in an inert solvent such as DCM, THF or dioxane optionally in the presence of a base such as DIPEA.

The reaction may be carried out at ambient temperature or at elevated temperatures using standard equipment or a single node microwave oven.

d) Synthesis of compounds of the general formula (XXX), 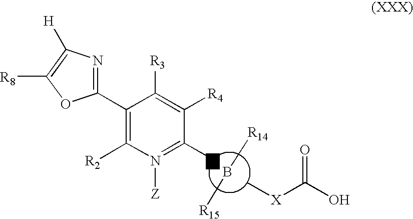
in which R₂, R₃, R₄, B, R₈, R₁₄ and R₁₅ are defined as in formula (I) above and X is a carbon, a single bond or (—CH₂—)ₙ (n=2-6) comprises the below steps. (d1-d5)

d1) Reacting the corresponding compounds of the general formula (XII) which is defined as above with a compound of the general formula (XXI) 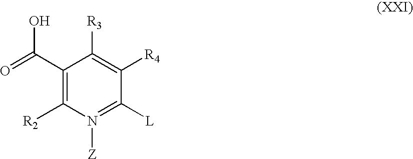
in which R₂, R₃ and R₄ are defined as in formula (I) above, and L is a suitable leaving group, such as chloro, bromo, iodo, triflate (OTf) or tosylate (OTs), to give a compound of formula (XXII).

The reactions are carried out at elevated temperatures using standard equipment or a single-node microwave oven. Optionally the reaction may be carried out in the presence of an organic base such as TEA or DIPEA.

d2) The compounds of formula (XXII) can then be reacted 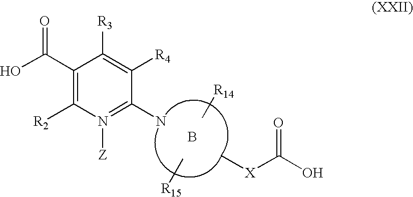
with a compound of the general formula (XXIII), 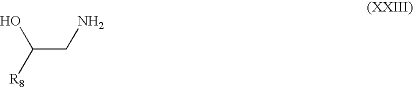
in which R₈ is defined as in formula (I) above, to give compounds of the general formula (XXIV). The reactions are carried out using standard conditions or in the presence of EDCI or the combination of EDCI and HOBT. Optionally the reaction may be carried out in the presence of an organic base such as TEA or DIPEA.

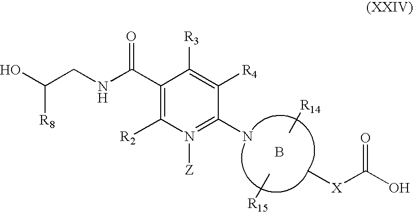

d3) This compound (XXIV) can then be transformed to a compound of the general formula (XX)

d4) The preparation of compounds with the general formula (XX), 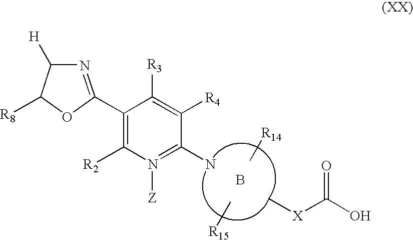
in which R₂, R₃, R₄, B, R₈, R₁₄ and R₁₅ are defined as in formula (I) above and X is a carbon, a single bond or (—CH₂—)ₙ (n=2-6) using known methods or a known reagent such as methanesulfonyl chloride. Optionally the reaction may be carried out in the presence of an organic base such as TEA.

d5) a compound of the general formula (XXX) as defined above can be made by oxidizing the corresponding compound of the general formula (XX) using a known oxidation reagent such as DDQ.

e) The preparation of compounds of the general formula (XXX) also comprises the steps (e1-e4) below;

e1) Reacting a compound the general formula (XXXI), 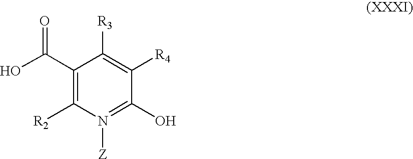
in which R₂, R₃ and R₄ are defined as in formula (I) above, with a compound of the general formula (XXXII), in which R₈ is defined as in formula (I) above, 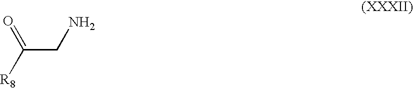
using standard conditions or in the presence of EDCI or the combination of EDCI and HOBT. Optionally the reaction may be carried out in the presence of an organic base such as TEA. This reaction gives a compound of the general formula (XXXIII).

e2) The compound of the general formula (XXXIII) obtained 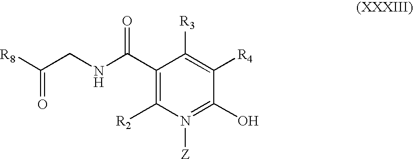
can then be transformed to a compound of the general formula (XXXIV), in which R₂, R₃, R₄ and R₈ are defined as in formula (I) above, using known techniques or using a known reagent such as POCl₃.

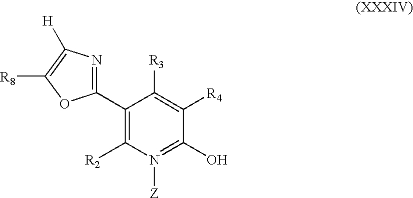

e3) A compound of the general formula (XXXIV) can then be transformed to a compound of the general formula (XXXV), 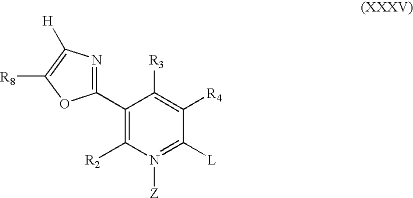
in which R₂, R₃, R₄, R₈ are defined as in formula (I) above and L is a sufficient leaving group, such as chloro, bromo, iodo, triflate (OTf) or tosylate (OTs), using a known techniques or a reagent such as oxalyl chloride or thionyl chloride.

e4) The compound of formula (XXXV) can then be reacted with a compound of the general formula (XII), which is defined as above, to give a compound of the general formula (XXX), defined as above. The reactions are carried out at elevated temperatures using standard equipment or a single-node microwave oven. Optionally the reactions may be carried out in the presence of an organic base such as TEA or DIPEA.

f) Preparation of Compounds of the General Formula (XXXVI), 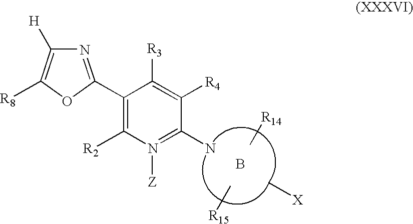
in which R₂, R₃, R₄, B, R₈, R₁₄ and R₁₅ are defined as in formula (I) above, X is a nitrogen, (—CH₂—NH₂) or a hydrogen that is connected to a nitrogen which is a member of the B ring, comprises the below steps. (f1-f4)

f1) Reacting a compound of the general formula (XIII) which is defined as above with a compound of the general formula (XXI) which is defined as above, to give a compound of the general formula (XXVIII).

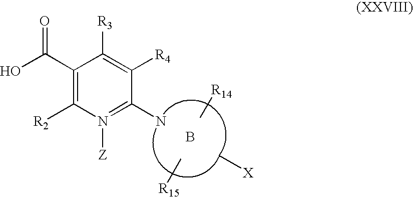

The reactions are carried out at elevated temperatures using standard equipment or a single-node microwave oven. Optionally the reaction may be carried out in the presence of an organic base such as TEA or DIPEA.

f2) The compound of formula (XXVIII) can be reacted with a compound of formula (XXIII), which is defined as above, to give compounds of the general formula (XXIX). The reactions are carried out using standard conditions or in the presence of EDCI or the combination of EDCI and HOBT. Optionally the reactions may be carried out in the presence of an organic base such as TEA or DIPEA.

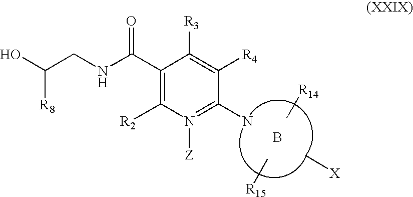

f3) This compound can then be transformed to a compound of the general formula (XXVI) in which R₂, R₃, R₄, B, R₈, R₁₄ and R₁₅, are defined as in formula (I) above, 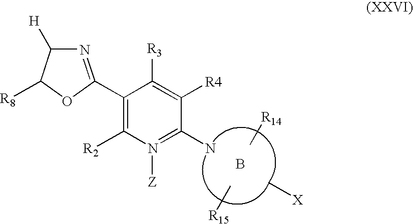
X is a nitrogen, (—CH₂—NH₂) or a hydrogen connected to a nitrogen which is a member of the B ring, using known methods or a sufficient reagent such as methanesulfonyl chloride. Optionally the reaction may be carried out in the presence of an organic base such as TEA.

f4) (XXXVI) can then be prepared by oxidizing a compound of the general formula (XXVI), which is defined as above. The reaction can be performed using standard conditions or a reagent like DDQ.

Compounds of the general formula (II), in which R₁ is R₇C(O) and R₂, R₃, R₄, R₇, B, R₁₄ and R₁₅ are defined as in formula (I) above, X is a single bond, a carbon or (—CH₂—)ₙ (n=2-6) comprises the following steps (g1-g2):

g1) Reacting a compound of the general formula (XXII), described above, with N,O-dimethylhydroxylamine. The reaction can be performed using known reagents like CDI, EDCI or the combination of EDCI and HOBT to give a compound of the general formula (XXXVIII).

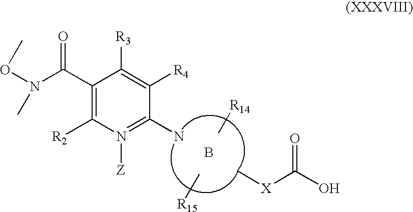

g2) Reacting compounds of the general formula (XXXVIII), defined as above, with a reagent of the general formula R₇—MgX′, in which R₇ is defined as in formula (I) above and X′ is a halogen, or a reagent of the formula R₇-M, in which M is a metal exemplified by Zn and Li.

Compounds of the general formula (IV), in which R₁ is R₇C(O) and R₂, R₃, R₄, R₇, B, R₁₄ and R₁₅ are defined as in formula (I) above, X is a nitrogen, (—CH₂—NH₂) or a hydrogen that is connected to a nitrogen which is a member of the B ring, comprises the following steps (h1-h2).

h1) Reacting a compound of the general formula (XXVIII), defined as above, with N,O-dimethylhydroxylamine. The reaction can be performed using known reagents like CDI, EDCI or the combination of EDCI and HOBT to give a compound of the general formula (XLI).

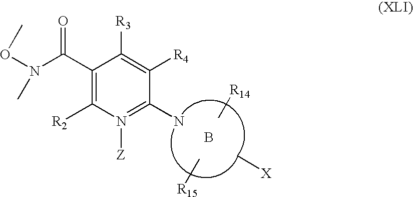

h2) A compound of the general formula (XLI), which is defined as above can be reacted with a reagent of the general formula R₇—MgX′, in which R₇ is defined as in formula (I) above and X′ is a halogen, or a reagent of the formula R₇-M, in which M is a metal exemplified by Zn and Li.

Compounds of the general formula (VIII) can be formed in one of the processes (i1-i4). The compounds of formula (VIII) in which R₅ is a hydrogen are advantageously isolated as a zwitterion. A ring nitrogen of compounds of formula (XII) and (XIII) used in the below steps may be protected by a protective group such as t-butyloxycarbonyl.

i1) Compounds of the general formula (VIII) in which B, R₅, R₁₄, R₁₅, R^{c} and R^{d} are defined as in formula (I) above, X is a single bond, a carbon or (—CH₂—)ₙ (n=2-6) may be formed by reacting a compound of formula (XII) with a compound of formula (III). The reaction is generally carried out in an inert organic solvent such as dichloromethane at ambient temperature. The reaction may be carried out using standard conditions or in the presence of EDCI or the combination of EDCI and HOBT. Optionally, the reaction may be carried out in the presence of an organic base such as triethylamine or DIPEA.

i2) Compounds of the general formula (VIII) in which R₅ is hydrogen, B, R₁₄, R₁₅, R^{c} and R^{d} are defined as in formula (I) above, X is a nitrogen, (—CH₂—NH—) or a single bond connected to a nitrogen which is a member of the B ring, can be formed by reacting a compound of formula (XIII) defined as above with a compound of formula (V), defined as above. The reaction is generally carried out in an inert solvent such as THF. The reaction may also be carried out in the presence of an organic base such as triethylamine or DIPEA.

i3) Compounds of the general formula (VIII) in which B, R₅, R₁₄, R₁₅, R^{c} and R^{d} defined as in formula (I) above, X is a nitrogen, (—CH₂—NH—) or a single bond connected to a nitrogen which is a member of the B ring, can also be formed by reacting a compound of formula (XIII) with a compound of formula (VI) which is defined as above. The reaction is generally carried out in a solvent such as DMA. This reaction may also be carried out in the presence of an organic base such as triethylamine or DIPEA

i4) A compound of formula (VIII) which is protected with t-butoxy carbonyl may be transformed into a compound without the protective group using standard procedures or a reagent such as HCl or TFA.

(j) Compounds of the general formula (VII) which are defined as above can be formed by reacting a compound of formula (XLVI) using standard conditions or with a halogenating reagent such as oxalyl chloride, thionyl chloride, POCl₃ or POBr₃. Advantageously dimethylformamide may be used as a catalyst for the reaction. The reaction may be performed in an inert solvent such as methylene chloride or toluene. Advantageously the inert solvent is toluene. Alternatively the reaction can be carried out using (Tf)₂O or TsCl preferably in the presence of a base such as DIPEA or triethylamine. The reaction may be performed in an inert solvent such as methylene chloride or THF. 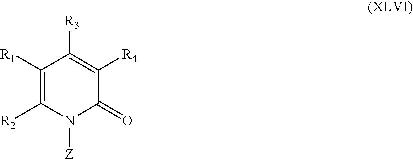
The preparation of compounds of the general formula (XLVII) which is defined as above comprises the steps (k1-k3) below;

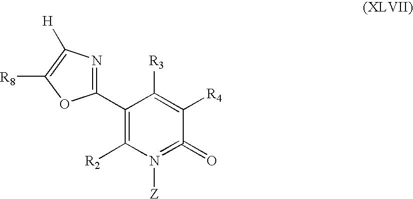

k1) Reacting a compound of the general formula (XLVIII) 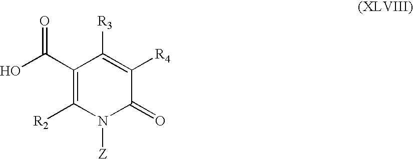
with a compound of the general formula (XXIII) defined as above, to give a compound of the formula (IL). The reaction is generally carried out in DCM at ambient temperature. The reaction may be carried out using standard conditions or in the presence of EDCI or the combination of EDCI and HOBT. Optionally the reaction may be carried out in the presence of an organic base such as TEA or DIPEA.

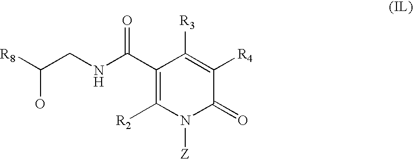

k2) The compound of formula (IL) can be transformed to a compound (L) using standard conditions or an oxidizing agent such as the mixture of oxalylchloride and DMSO.

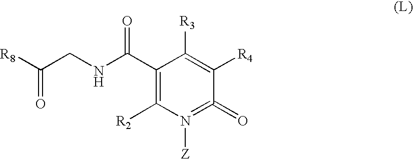

k3) The compound of formula (L) can then be transformed into a compound of the general formula (XLVII), using standard conditions or in the presence of (Methoxycarbonylsulfamoyl)triethylammonium hydroxide (Burgess reagent). The reaction is generally performed in an inert solvent such as THF. The reaction is carried out at elevated temperatures using standard equipment or a single-node microwave oven.

l) Preparation of compounds of the general formula (XLVIII) which is defined as above except for R₃ which is hydrogen, comprises the following steps (l1-l3);

l1) Reacting a compound of the formula (LI), in which R₂ and R₆ are defined as in formula (I) above with dimethoxy-N,N-dimethylmethaneamine to form a 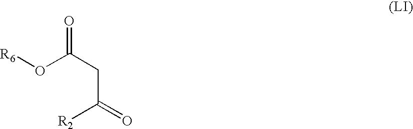
compound of formula (LII).

l2) This compound (LII) can then be reacted further with a compound of the 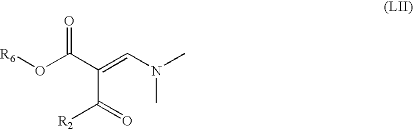
general formula R₄CH₂C(O)NH₂, in which R₄ is defined as in formula (I) above to give a compound of the general formula (LIII). The reaction is generally performed in an inert solvent such as ethanol, optionally in the presence of a strong base such as sodium ethoxide.

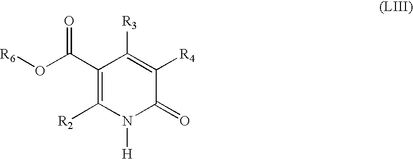

l3) A compound of the general formula (LIII) can then be transformed to a compound of the general formula (XLVIII). The reaction is generally performed in a protic solvent such as water together with a co-solvent such as THF or methanol. The reaction can be performed using standard reagents or in the presence of LiOH, NaOH or KOH.

(m) The formation of a compound of the general formula (XXX), which is defined as above can be made the below synthesis;

m1) A compound of the general formula (LIV) where R₈ is defined as in formula (I) above can be 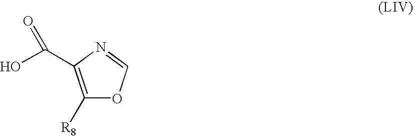
transformed in to a compound of the formula (LV) 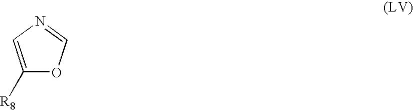
using standard conditions or using Cu(II)O and quinoline.

m2) The compound of the general formula (LV) can be reacted with a compound of the general formula (LVI) in 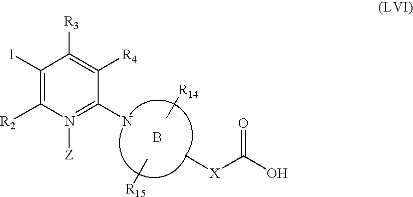
which R₂, R₃, R₄, B, R₁₄ and R₁₅ are defined as for formula (I) and X is a carbon, a single bond or (—CH₂—)ₙ (n=2-6), to give compounds of the general formula (XXX). The reaction is generally performed in an inert solvent such as THF under inert atmosphere. The reaction can be performed using standard conditions or in the presence of AlkylLi such as BuLi followed by treatment with ZnCl₂ and Pd(PPh₃)₄ (preferably a catalytic amount).

(n) Compounds of the general formula (XXXVI) can also be made by the step below;

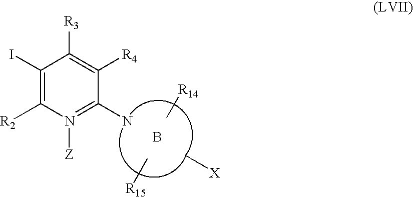

n1) Reacting a compound of the general formula (LV), which is defined as above, with a compound of the general formula (LVII), in which R₂, R₃, R₄, B, R₁₄ and R₁₅ are defined as in formula (I) above, X is a nitrogen, (—CH₂—NH₂) or a hydrogen that is connected to a nitrogen which is a member of the B ring. The reaction can be performed using standard conditions or in the presence of AlkylLi such as BuLi followed by treatment with ZnCl₂ and Pd(PPh₃)₄ (preferably a catalytic amount).

o) Compounds of the general formula (IX) wherein X, B, R₁₄, R₁₅, R₅, R^{c} and R^{d} are defined as in formula (I), R₁ is R₆OC(O), R₃ is H, R₄ is CN, Z is absent can be prepared by the following steps o1-o2 below

o1) Reacting a compound of the general formula (LVIII) 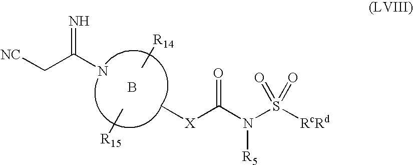
where R₅, B, R₁₄, R₁₅, X, R^{c} and R^{d} are as defined in formula (I) above with a compound of formula (LIX) 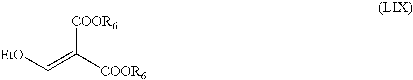
The reaction is generally carried out in an inert organic solvent such as EtOH or DMSO. The reaction is carried out at ambient temperature or at elevated temperatures using standard equipment or a single node microwave oven.

o2) Compounds of the general formula (LVIII) defined above can be prepared by reacting a compound of the general formula (VIII) as defined above with a compound of formula (LX) 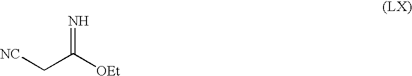
using essentially the same procedure as described in Macconi, A et. Al., J. Heterocyclic chemistry, 26, p. 1859 (1989).

o3) Compounds of general formula (IX) above wherein B, R₁₄, R₁₅, R₅, R^{c} and R^{d} are defined as in formula (I), R₁ is R₆OC(O), R₃ is H, R₄ is CN, Z is absent and X is a single bond, a carbon atom or (—CH₂—)ₙ (n=2-6) may be prepared by reacting a compound of formula (IIB) wherein B, R₁₄, R₁₅, are defined as in formula (I), R₁ is R₆OC(O), R₃ is H, R₄ is CN, Z is absent and X is a single bond, a carbon atom or (—CH₂)ₙ (n=2-6) 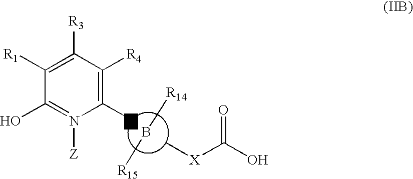
with a compound of formula (III) defined as above.

The reaction is generally carried out in an inert organic solvent such as dichloromethane at ambient temperature. The reaction may be carried out using standard conditions or in the presence of TBTU, EDCI, PyBrop or the combination of EDCI and HOBT. Optionally, the reaction may be carried out in the presence of an organic base such as triethylamine or DIPEA.

o4) Compounds of general formula (IIB) wherein B, R₁₄, and R₁₅ are defined as in formula (I), R₁ is R₆OC(O), R₃ is H, R₄ is CN, Z is absent and X is a single bond, a carbon atom or (—CH₂—)ₙ (n=2-6) may be prepared by reacting a compound of general formula (IIC) 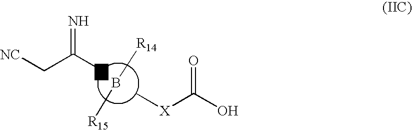
wherein R₁₄, R₁₅, and B is defined as in formula (I) and X is a single bond, a carbon atom or (—CH₂—)ₙ (n=2-6) with a compound of formula (LIX) defined as above.

The reaction is generally carried out in an inert organic solvent such as EtOH or DMSO.

The reaction is carried out at ambient temperature or at elevated temperatures using standard equipment or a single node microwave oven.

o5) Compounds of the general formula (IIC) defined above can be prepared by reacting a compound of the general formula (XII) as defined above with a compound of formula (LX) using essentially the same procedure as described in Macconi, A et. Al., J. Heterocyclic chemistry, 26, p. 1859 (1989).

o6) Compounds of general formula (IX) above wherein B, R₁₄, R₁₅, R₅, R^{c} and R^{d} are defined as in formula (I), R₁ is R₆OC(O), R₃ is H, R₄ is CN, Z is absent and X is a nitrogen, (—CH₂—NH—) or a single bond connected to a nitrogen which is a member of the B ring may be prepared by reacting a compound of formula (IVB) 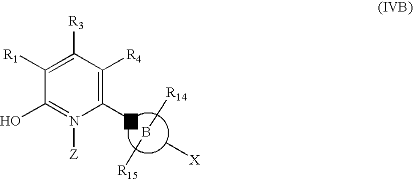
wherein B, R₁₄, R₁₅, are defined as in formula (I), R₁ is R₆OC(O), R₃ is H, R₄ is CN, Z is absent and X is a nitrogen, (—CH₂—NH₂) or a hydrogen that is connected to a nitrogen which is a member of the B ring with a compound of formula (III) defined as above.

The reaction is generally carried out in an inert solvent such as DCM. The reaction may be carried out in the presence of CDI. Optionally, the reaction may be carried out in the presence of an organic base such as triethylamine, DBU or DIPEA.

o7) Compounds of general formula (IX) above wherein B, R₁₄, R₁₅, R^{c} and R^{d} are defined as in formula (I), R₁ is R₆OC(O), R₃ is H, R₄ is CN, Z is absent, R₅ is hydrogen and X is a nitrogen, (—CH₂—NH—) or a single bond connected to a nitrogen which is a member of the B ring may be prepared by reacting a compound of formula (IVB) defined as in o6) above with a compound of general formula (V) defined above.

The reaction is generally carried out in an inert solvent such as THF. Optionally, the reaction may be carried out in the presence of an organic base such as triethylamine or DIPEA.

o8) Compounds of general formula (IX) above wherein B, R₁₄, R₁₅, R₅, R^{c} and R^{d} are defined as in formula (I), R₁ is R₆OC(O), R₃ is H, R₄ is CN, Z is absent, and X is a nitrogen, (—CH₂—NH—) or a single bond connected to a nitrogen which is a member of the B ring may be prepared by reacting a compound of formula (IVB) defined as in o6) above with a compound of general formula (VI) as defined above.

The reaction is generally carried out in an inert solvent such as DMA. Optionally, the reaction may be carried out in the presence of an organic base such as triethylamine or DIPEA.

o9) Compounds of the general formula (IVB) wherein B, R₁₄, R₁₅, are defined as in formula (I), R₁ is R₆OC(O), R₃ is H, R₄ is CN, Z is absent and X is a nitrogen, (—CH₂—NH₂) or a hydrogen that is connected to a nitrogen which is a member of the B ring may be prepared by essentially the same procedure described in steps o4)-o5) above from a compound of formula (XIII).

p1) Compounds of the general formula (IIA) defined as above may be prepared by reacting a compound of formula (IIB) above in which R₁, R₃, R₄, B, Z, R₁₄, and R₁₅ are defined as in formula (I) above, X is a single bond, a carbon or (—CH₂—)ₙ (n=2-6) using standard conditions or with a halogenating reagent such as oxalyl chloride, thionyl chloride, POCl₃ or POBr₃. Advantageously DMF may be used as a catalyst for the reaction. The reaction may be performed in an inert solvent such as methylene chloride or toluene. Alternatively the reaction can be carried out using (Tf)₂O or TsCl preferably in the presence of a base such as DIPEA or triethylamine. The reaction may be performed in an inert solvent such as methylene chloride or THF.

p2) Compounds of the general formula (IVA) defined as above may be prepared by reacting a compound of formula (IVB) wherein R₁, R₃, R₄, B, Z, R₁₄, R₁₅, are defined as in formula (I), and X is a nitrogen, (—CH₂—NH₂) or a hydrogen that is connected to a nitrogen which is a member of the B ring using standard conditions or with a halogenating reagent such as oxalyl chloride, thionyl chloride, POCl₃ or POBr₃. Advantageously DMF may be used as a catalyst for the reaction. The reaction may be performed in an inert solvent such as methylene chloride or toluene.

Alternatively the reaction can be carried out using (Tf)₂O or TsCl preferably in the presence of a base such as DIPEA or triethylamine. The reaction may be performed in an inert solvent such as methylene chloride or THF.

Compounds of formula (IXA) may be prepared by the following processes q1-q4:

q1) Compounds the of general formula (IXA) defined as above can be made by reacting a compound of formula (IX) defined as above using standard conditions or with a halogenating reagent such as oxalyl chloride, thionyl chloride, POCl₃ or POBr₃. Advantageously DMF may be used as a catalyst for the reaction. The reaction may be performed in an inert solvent such as methylene chloride or toluene.

Alternatively the reaction can be carried out using (Tf)₂O or TsCl preferably in the presence of a base such as DIPEA or triethylamine. The reaction may be performed in an inert solvent such as methylene chloride or THF.

q2) Compounds of the general formula (IXA) wherein and R₁, R₃, R₄, B, Z, R₅, R₆, R₁₄, R₁₅, R^{c} and R^{d} are as defined in formula (I) and X is a single bond, a carbon or (—CH₂—)ₙ (n=2-6) can be made by reacting a compound of formula (IIA) above with a compound of formula (III).

The reaction is generally carried out in an inert organic solvent such as dichloromethane at ambient temperature. The reaction may be carried out using standard conditions or in the presence of PyBrop, TBTU, EDCI or the combination of EDCI and HOBT. Optionally, the reaction may be carried out in the presence of an organic base such as triethylamine or DIPEA.

q3) Compounds of the general formula (IXA) wherein and R₁, R₃, R₄, B, Z, R₆, R₁₄, R₁₅, R^{c} and R^{d} are as defined in formula (I) and X is a nitrogen, (—CH₂—NH—) or a single bond connected to a nitrogen which is a member of the B ring can be formed by reacting a compound of formula (IVA) with a compound of formula (V) defined as above.

The reaction is generally carried out in an inert solvent such as THF. Optionally, the reaction may be carried out in the presence of an organic base such as triethylamine or DIPEA.

q4) Compounds of the general formula (IXA) wherein and R₁, R₃, R₄, B, Z, R₅, R₆, R₁₄, R₁₅, R^{c} and R^{d} are as defined in formula (I) and X is a nitrogen, (—CH₂—NH—) or a single bond connected to a nitrogen which is a member of the B ring, can be formed by reacting a compound of formula (IV) with a compound of formula (VI) defined as above.

The reaction is generally carried out in a solvent such as DMA. Optionally, the reaction may be carried out in the presence of an organic base such as triethylamine or DIPEA.

r) The preparation of compounds of the general formula (LXI), in which R₁₄ and R₁₅ are defined as for formula (I) with the exception that R₁₄ is connected to the same atom as X, and X is defined as a single bond, comprises the below step;

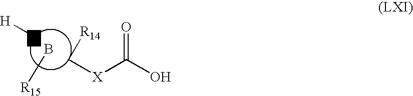

r1) Reacting the corresponding (LXII) with R₁₄-L, wherein L is a suitable leaving group, such as chloro, bromo, iodo, 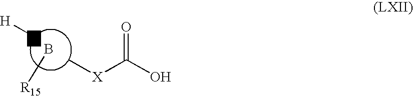
triflate (OTf) or tosylate (OTs) to form compounds of the general formula (LXI), using standard conditions or in the presence of a mixture of BuLi and diisopropylamine (to form LDA).

The preparation of compounds of the formula (III) comprises the below processes. (s1-s3)

s1) A compound of the formula LR^{c}R^{d} wherein L is a suitable leaving group, such as chloro, bromo, iodo could be transformed to the corresponding compound (III) using a sequence of reactions using first SMOPS (Baskin and Wang. Tetrahedron Letters, 2002, 43, 8479-83. See esp. page 8480, left hand column) followed by hydrolysis using a base like NaOMe in an inert solvent like DMSO at room temperature. Followed by treatment by NH₂OSO₃H and NaOAc to give a compound of formula (III).

s2) A compound of the formula LSO₂R^{c}R^{d} wherein L is a suitable leaving group, such as chloro, bromo, iodo could be reacted with ammonium hydroxide or H₂NR₅ in an inert solvent such as DCM to give a compound of formula (III).

s3) A compound of the formula LR^{c}R^{d} wherein L is a suitable leaving group, such as chloro, bromo, iodo could be transformed to the corresponding compound (III) using a sequence of reactions first NaSO₃, followed by a using a reagent such as PCl₅, POCl₃ or SOCl₂, followed by ammonium hydroxide or H₂NR₅ to give a compound of formula (III).

At any stage in the synthesis of amine substituted pyridines, a halogen substituent in the 2, 4 or 6 position of the pyridine can be substituted with azide using known techniques. The azide can be reduced to the corresponding amine. These amines can subsequently be alkylated or acylated using known methods or with an alkylhalide or acylhalide, respectively.

Persons skilled in the art will appreciate that an acid can be transformed to the corresponding activated ester such as an acid chloride, followed by reaction with a thiol, R₁₆SH to give thioesters, R₁₆SC(O).

Persons skilled in the art will appreciate that an acid can be transformed to the corresponding activated ester such as an acid chloride, followed by reaction with a alcohol, R₆OH to give esters, R₆OC(O).

Persons skilled in the art will appreciate that a compound of formula (III) could be alkylated at the carbon atom in the alpha position to the sulfonamide using an alkylhalide. Preferably under basic conditions using a strong base such as sodium hydride.

Persons skilled in the art will appreciate that a nitrogen substituent at the 3 position of a pyridine could be replaced by a thioether chain, R₁₇S—, using known techniques or R₁₇SSR₁₇ and tert-Butylnitrite.

Persons skilled in the art will appreciate that a thioketone could be made from the corresponding ketone using known techniques or using Lawessons reagent.

Persons skilled in the art will appreciate that a pyridine N-oxide could be formed by from a pyridine using an oxidizing agent such as Urea hydrogen peroxide or hydrogen peroxide, with or without the presence of trifluoroaceticanhydride.

The compounds of the invention may be isolated from their reaction mixtures using conventional techniques.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

It will be appreciated that by those skilled in the art that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldiphenylsilyl or trimethylsilyl) and tetrahydropyranyl. Suitable protecting groups for carboxylic acids include (C₁-C₆)alkyl or benzyl esters. Suitable protecting groups for amino include allyl, t-butyloxycarbonyl, benzyloxycarbonyl, 2-(trimethylsilyl)ethoxymethyl or 2-trimethylsilylethoxycarbonyl (Teoc).

The protection and deprotection of functional groups may take place before or after any reaction in the above mentioned processes.

Persons skilled in the art will appreciate that, in order to obtain compounds of the invention in an alternative, and on some occasions, more convenient, manner, the individual process steps mentioned hereinbefore may be performed in different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

Persons skilled in the art will appreciate that starting materials for any of the above processes can in some cases be commercially available.

Persons skilled in the art will appreciate that processes above could for some starting materials above be found in the general common knowledge.

The type of chemistry involved will dictate the need for protecting groups as well as sequence for accomplishing the synthesis.

The use of protecting groups is fully described in “Protective groups in Organic Chemistry”, edited by J W F McOmie, Plenum Press (1973), and “Protective Groups in Organic Synthesis”, 3^{rd} edition, T. W. Greene & P. G. M Wutz, Wiley-Interscience (1999). Protected derivatives of the invention may be converted chemically to compounds of the invention using standard deprotection techniques (e.g. under alkaline or acidic conditions). The skilled person will also appreciate that certain compounds of Formula (II)-(LXII) may also be referred to as being “protected derivatives.”

Compounds of the invention may also contain one or more asymmetric carbon atoms and may therefore exhibit optical and/or diastereoisomerism. Diastereoisomers may be separated using conventional techniques, e.g. chromatography or crystallization. The various stereisomers may be isolated by separation of a racemic or other mixture of the compounds using conventional, e.g. HPLC techniques. Alternatively the desired optical isomers may be made by reaction of the appropriate optically active starting materials under conditions which will not cause racemisation or epimerization, or by derivatisation, for example with a homochiral acid followed by separation of the diasteromeric derivatives by conventional means (e.g. HPLC, chromatography over silica or crystallization). Stereo centers may also be introduced by asymmetric synthesis, (e.g. metalloorganic reactions using chiral ligands). All stereoisomers are included within the scope of the invention.

All novel intermediates form a further aspect of the invention.

Salts of the compounds of formula (I) may be formed by reacting the free acid, or a salt thereof, or the free base, or a salt or a derivative thereof, with one or more equivalents of the appropriate base (for example ammonium hydroxide optionally substituted by C₁-C₆-alkyl or an alkali metal or alkaline earth metal hydroxide) or acid (for example a hydrohalic (especially HCl), sulphuric, oxalic or phosphoric acid). The reaction may be carried out in a solvent or medium in which the salt is insoluble or in a solvent in which the salt is soluble, e.g. water, ethanol, tetrahydrofuran or diethyl ether, which may be removed in vacuo, or by freeze drying. The reaction may also carried out on an ion exchange resin. The non-toxic physiologically acceptable salts are preferred, although other salts may be useful, e.g. in isolating or purifying the product.

Pharmacological Data

Functional inhibition of the P2Y₁₂ receptor can be measured by in vitro assays using cell membranes from P2Y₁₂ transfected CHO-cells, the methodology is indicated below.

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintillant and counted for the amount of ³⁵S-GTPγS retained by the filter. Maximum activity was that determined in the presence of the agonist and minimum activity in the absence of the agonist following subtraction of the value determined for non-specific activity. The effect of compounds at various concentrations was plotted according to the equation
y=A+((B−A)/(1+((C/x)ˆD)))
and IC₅₀ estimated where

A is the bottom plateau of the curve i.e. the final minimum y value

B is the top of the plateau of the curve i.e. the final maximum y value

C is the x value at the middle of the curve. This represents the log EC₅₀ value when A+B=100

D is the slope factor

x is the original known x values

Y is the original known y values.

Most of the compounds of the invention have an activity, when tested in the functional inhibition of 2-Me-S-ADPinduced P2Y₁₂ signalling assay described, at a concentration of around 4 μM or below.

For example the compounds described in Examples 41 and 74 gave the following test result in the functional inhibition of 2-Me-S-ADPinduced P2Y₁₂ signalling assay described.

| | IC₅₀(μM) |
|---|---|
| Example 41 | 0.49 |
| Example 74 | 0.27 |

The compounds of the invention act as P2Y₁₂ receptor antagonists and are therefore useful in therapy. Thus, according to a further aspect of the invention there is provided a compound of formula (I), or a pharmaceutically acceptable salt thereof, for use in therapy.

In a further aspect there is provided the use of a compound of formula (I), or a pharmaceutically acceptable salt thereof, for the manufacture of a medicament for treatment of a platelet aggregation disorder. In another aspect of the invention there is provided the use of a compound of formula (I), or a pharmaceutically acceptable salt thereof, for the manufacture of a medicament for the inhibition of the P2Y₁₂ receptor.

The compounds are useful in therapy, especially adjunctive therapy, particularly they are indicated for use as: inhibitors of platelet activation, aggregation and degranulation, promoters of platelet disaggregation, anti-thrombotic agents or in the treatment or prophylaxis of unstable angina, coronary angioplasty (PTCA), myocardial infarction, perithrombolysis, primary arterial thrombotic complications of atherosclerosis such as thrombotic or embolic stroke, transient ischaemic attacks, peripheral vascular disease, myocardial infarction with or without thrombolysis, arterial complications due to interventions in atherosclerotic disease such as angioplasty, endarterectomy, stent placement, coronary and other vascular graft surgery, thrombotic complications of surgical or mechanical damage such as tissue salvage following accidental or surgical trauma, reconstructive surgery including skin and muscle flaps, conditions with a diffuse thrombotic/platelet consumption component such as disseminated intravascular coagulation, thrombotic thrombocytopenic purpura, hemolytic uraemic syndrome, thrombotic complications of septicemia, adult respiratory distress syndrome, anti-phospholipid syndrome, heparin-induced thrombocytopenia and pre-eclampsia/eclampsia, or venous thrombosis such as deep vein thrombosis, venoocclusive disease, haematological conditions such as myeloproliferative disease, including thrombocythemia, sickle cell disease; or in the prevention of mechanically-induced platelet activation in vivo, such as cardio-pulmonary bypass and extracorporeal membrane oxygenation (prevention of microthromboembolism), mechanically-induced platelet activation in vitro, such as use in the preservation of blood products, e.g. platelet concentrates, or shunt occlusion such as in renal dialysis and plasmapheresis, thrombosis secondary to vascular damage/inflammation such as vasculitis, arteritis, glomerulonephritis, inflammatory bowel disease and organ graft rejection, conditions such as migraine, Raynaud's phenomenon, conditions in which platelets can contribute to the underlying inflammatory disease process in the vascular wall such as atheromatous plaque formation/progression, stenosis/restenosis and in other inflammatory conditions such as asthma, in which platelets and platelet-derived factors are implicated in the immunological disease process.

According to the invention there is further provided the use of a compound according to the invention in the manufacture of a medicament for the treatment of the above disorders. In particular the compounds of the invention are useful for treating myocardial infarction, thrombotic stroke, transient ischaemic attacks, peripheral vascular disease and angina, especially unstable angina. The invention also provides a method of treatment of the above disorders which comprises administering to a patient suffering from such a disorder a therapeutically effective amount of a compound according to the invention.

In a further aspect the invention provides a pharmaceutical composition comprising a compound of formula (I), or a pharmaceutically acceptable salt thereof, together with a pharmaceutically acceptable diluent, adjuvant and/or carrier.

The compounds may be administered topically, e.g. to the lung and/or the airways, in the form of solutions, suspensions, HFA aerosols and dry powder formulations; or systemically, e.g. by oral administration in the form of tablets, pills, capsules, syrups, powders or granules, or by parenteral administration in the form of sterile parenteral solutions or suspensions, by subcutaneous administration, or by rectal administration in the form of suppositories or transdermally.

The compounds of the invention may be administered on their own or as a pharmaceutical composition comprising the compound of the invention in combination with a pharmaceutically acceptable diluent, adjuvant or carrier. Particularly preferred are compositions not containing material capable of causing an adverse, e.g. an allergic, reaction. Dry powder formulations and pressurised HFA aerosols of the compounds of the invention may be administered by oral or nasal inhalation. For inhalation the compound is desirably finely divided. The compounds of the invention may also be administered by means of a dry powder inhaler. The inhaler may be a single or a multi dose inhaler, and may be a breath actuated dry powder inhaler.

One possibility is to mix the finely divided compound with a carrier substance, e.g. a mono-, di- or polysaccharide, a sugar alcohol or another polyol. Suitable carriers include sugars and starch. Alternatively the finely divided compound may be coated by another substance. The powder mixture may also be dispensed into hard gelatine capsules, each containing the desired dose of the active compound.

Another possibility is to process the finely divided powder into spheres, which break up during the inhalation procedure. This spheronized powder may be filled into the drug reservoir of a multidose inhaler, e.g. that known as the Turbuhaler® in which a dosing unit meters the desired dose which is then inhaled by the patient. With this system the active compound with or without a carrier substance is delivered to the patient.

The pharmaceutical composition comprising the compound of the invention may conveniently be tablets, pills, capsules, syrups, powders or granules for oral administration; sterile parenteral or subcutaneous solutions, suspensions for parenteral administration or suppositories for rectal administration.

For oral administration the active compound may be admixed with an adjuvant or a carrier, e.g. lactose, saccharose, sorbitol, mannitol, starches such as potato starch, corn starch or amylopectin, cellulose derivatives, a binder such as gelatine or polyvinylpyrrolidone, and a lubricant such as magnesium stearate, calcium stearate, polyethylene glycol, waxes, paraffin, and the like, and then compressed into tablets. If coated tablets are required, the cores, prepared as described above, may be coated with a concentrated sugar solution which may contain e.g. gum arabic, gelatine, talcum, titanium dioxide, and the like. Alternatively, the tablet may be coated with a suitable polymer dissolved either in a readily volatile organic solvent or an aqueous solvent.

For the preparation of soft gelatine capsules, the compound may be admixed with e.g. a vegetable oil or polyethylene glycol. Hard gelatine capsules may contain granules of the compound using either the above mentioned excipients for tablets, e.g. lactose, saccharose, sorbitol, mannitol, starches, cellulose derivatives or gelatine. Also liquid or semisolid formulations of the drug may be filled into hard gelatine capsules.

Liquid preparations for oral application may be in the form of syrups or suspensions, for example solutions containing the compound, the balance being sugar and a mixture of ethanol, water, glycerol and propylene glycol. Optionally such liquid preparations may contain colouring agents, flavouring agents, saccharine and carboxymethylcellulose as a thickening agent or other excipients known to those skilled in art.

The invention will be further illustrated with the following non-limiting examples:

### EXAMPLES

### General Experimental Procedure

Mass spectra was recorded on a Finnigan LCQ Duo ion trap mass spectrometer equipped with an electrospray interface (LC-ms) or LC-ms system consisting of a Waters ZQ using a LC-Agilent 1100 LC system. ¹H NMR measurements were performed on a Varian Mercury VX 400 spectrometer, operating at a 1H frequency of 400 and Varian UNITY plus 400, 500 and 600 spectrometers, operating at 1H frequencies of 400, 500 and 600 respectively. Chemical shifts are given in ppm with the solvent as internal standard. Protones on heteroatoms such as NH and OH protons are only reported when detected in NMR and can therefore be missing. Chromatography was performed using Biotage silica gel 40S, 40M, 12i or Merck silica gel 60 (0.063-0.200 mm). Flashchromatography was performed using either standard glass- or plastic-columns column or on a Biotage Horizon system. HPLC separations were performed on a Waters YMC-ODS AQS-3 120 Angstrom 3×500 mm or on a Waters Delta Prep Systems using Kromasil C8, 10 μm columns.

The purification system and LC-MS system used in Method A to E below was Waters Fraction Lynx II Purification System: Column: Sunfire Prep C18, 5 μm OBD, 19×100 mm columns Gradient 5-95% CH₃CN in 0.1 mM HCOOH (pH=3). MS triggered fraction collection was used. Mass spectra were recorded on either Micromass ZQ single quadropole or a Micromass quattro micro, both equipped with a pneumatically assisted electrospray interface.

Reactions performed in a microwave reactor were performed in a Personal Chemistry Smith Creator, Smith synthesizer or an Emrys Optimizer.

### LIST OF USED ABBREVIATIONS

| Abbreviation | Explanation |
|---|---|
| AcOH | Acetic acid |
| aq | Aqueous |
| br | Broad |
| Brine | A saturated solution of sodium chloride in water |
| BSA | Bovine Serum Albumine |
| (Boc)₂O | di-tert-butyl dicarbonate |
| BuLi | Butyl lithium |
| CDI | Carbonyldiimidazole |
| d | Doublet |
| DBU | 1,8-Diazabicyclo[5.4.0]undec-7-ene |
| DCM | Dichloromethane |
| DDQ | 2,3-Dichloro-5,6-dicyano-1,4-benzoquinone |
| DIPEA | N,N-Diisopropylethylamine |
| DMA | N,N-Dimethylacetamide |
| DMAP | N,N-dimethylpyridin-4-amine |
| DMF | N,N-dimethylformamide |
| DMSO | Dimethylsulphoxide |
| EDCI | N-[3-(dimethylamino)propyl]-N′-ethylcarbodiimide |
| | hydrochloride |
| EtOAc | Ethyl acetate |
| EtOH | Ethanol |
| h | hours |
| HATU | O-(7-Azabenzotriazol-1-yl)-1,1,3,3-tetramethyluromium |
| | hexafluorophosphate |
| HEPES | [4-(2-hydroxyethyl)-1-piperazineethanesulfonic |
| | acid |
| HFA | Hydrofluoroalkanes |
| HOAc | Acetic acid |
| HOBT | 1-Hydroxybenzotriazole |
| HPLC | High-performance liquid chromatography |
| Hz | Hertz |
| IPA | isopropyl alcohol |
| J | Coupling constant |
| LDA | Litiumdiisopropyl amide |
| m | Multiplet |
| Me | methyl |
| MHz | Megahertz |
| min | Minutes |
| mL | Millilitre |
| MS | Mass spectra |
| NCS | N-chlorosuccinimide |
| OAc | acetate |
| ⁱPrOAc | iso-propyl acetate |
| PyBrop | Bromo(tripyrrolidin-1-yl)phosphonium |
| | hexafluorophosphate |
| q | Quartet |
| r.t | Room temperature |
| s | Singlet |
| t | triplet |
| TB | Tyrodes Buffer |
| TBDMSC1 | tert-butyl(chloro)dimethylsilane |
| TBME | tert-butylmethyl ether |
| TBTU | N-[(1H-1,2,3-benzotriazol-1- |
| | yloxy)(dimethylamino)methylene]-N- |
| | methylmethanaminium tetrafluoroborate |
| TEA | Triethylamine |
| Tf | trifluoromethylsulfonyl |
| TFA | Trifluoroacetic acid |
| THF | Tetrahydrofurane |
| TMEDA | N,N,N′,N′-tetramethylethylendiamine |
| Ts | p-toluenesulfonyl |

Synthesis of Sulfone Amides

The synthesis of the sulfonamides used in the examples below was made with one of the three methods described below:

i) By reacting the corresponding sulfonyl chloride with ammonia in THF or MeOH or by treatment with ammonium hydroxide in methylene chloride. The sulfonamides obtained was used without further purification.

ii) By essentially following the procedure described by Seto, T. et. al. in J. Organic Chemistry, Vol 68, No 10 (2003), pp. 4123-4125; or

iii) By essentially following the procedure described by Wang, Z et. al. in Tetrahedron Letters, Vol 43 (2002), pp 8479-8483.

### Synthesis of Examples

The following general procedures (i.e. Method A to E) were used to prepare some of the examples below and are referred to in each specific example.

### Method A

### Exemplified by the Procedure from Example 10

DIPEA (64 mg, 0.5 mmol) was added to a solution of 1-[3-cyano-6-(difluoromethyl)-5-(ethoxycarbonyl)pyridin-2-yl]piperidine-4-carboxylic acid (35.3 mg, 0.1 mmol) and TBTU (38.5 mg, 0.12 mmol) in DCM (5 ml) and the mixture was stirred for 30 min at R.T. before 1-(2-fluorophenyl)methanesulfonamide (23 mg, 0.12 mmol) dissolved in dcm (1 ml) was added. The reaction was allowed to stir over night. LC-MS showed that starting material was left and more TBTU (19 mg, 0.06 mmol) and DIPEA (26 mg, 0.2 mmol) were added to the mixture and the stirring was continued for another 2 h. The reaction mixture was washed with 1% KHSO₄, the aqueous phase was extracted with DM (1 ml) and the combined organic phase was passed through a phase separator and evaporated in a vacuum centrifuge. The crude product obtained was purified by HPLC (see general experimental procedure) to give ethyl 5-cyano-2-(difluoromethyl)-6-[4-({[(2-fluorobenzyl)sulfonyl]amino}carbonyl)piperidin-1-yl]nicotinate. Yield: 41 mg (78%).

### Method B

### Exemplified by the Procedure from Example 42

DIPEA (128 mg, 1.0 mmol) was added to a solution of {1-[3-cyano-5-(ethoxycarbonyl)-6-(trifluoromethyl)pyridin-2-yl]pyrrolidin-3-yl}acetic acid (74.2 mg, 0.2 mmol) and TBTU (77 mg, 0.24 mmol) in DCM (7 mL) and the mixture was stirred for 30 min at r.t before 1-phenylmethanesulfonamide (41 mg, 0.24 mmol) dissolved in DCM (1 mL) was added and the reaction was left over night. The reaction mixture was washed with 1% KHSO₄, the aqueous phase was extracted with DCM and the combined organic phase was passed through a phase separator and evaporated in vacuum centrifuge. The crude product obtained was purified by HPLC (See General Experimental Procedure) to give ethyl 6-(3-{2-[(benzylsulfonyl)amino]-2-oxoethyl}pyrrolidin-1-yl)-5-cyano-2-(trifluoromethyl)nicotinate. Yield: 88 mg (84%).

### Method C

### Exemplified by the Procedure from Example 55

DIPEA (43 mg, 0.3 mmol) and TBTU (64 mg, 0.20 mmol) was added to a solution of 1-[3-cyano-5-(ethoxycarbonyl)-6-(trifluoromethyl)pyridin-2-yl]piperidine-4-carboxylic acid (74.2 mg, 0.2 mmol) in DMF and the mixture was stirred for 2 hours at r.t before it was added to 1-(4-fluorophenyl)methanesulfonamide (38 mg, 0.22 mmol) dissolved in DMF. The reaction mixture was stirred over night and passed through SCX-2 ion exchange column. The crude product obtained was purified by HPLC (See General Experimental Procedure) to give ethyl 5-cyano-6-[4-({[(4-fluorobenzyl)sulfonyl]amino}carbonyl)piperidin-1-yl]-2-(trifluoromethyl)nicotinate. Yield: 4.3 mg (4%).

### Method D

### Exemplified by the Procedure from Example 45

CDI (26 mg, 0.16 mmol) was added to a solution of 1-[3-cyano-5-(ethoxycarbonyl)-6-(trifluoromethyl)pyridin-2-yl]azetidine-3-carboxylic acid (51 mg, 0.15 mmol) (gas evolution) in CH₃CN and the mixture was heated to 50° C. for 2 hours. The above mixture was then added to a solution of 1-(4-fluorophenyl)methanesulfonamide (28 mg, 0.15 mmol) and DBU (23 mg, 0.15 mmol) in CH₃CN and the reaction was stirred at r.t over night. Purification by HPLC (See General Experimental Procedure) gave ethyl 5-cyano-6-[3-({[(4-fluorobenzyl)sulfonyl]amino}carbonyl)azetidin-1-yl]-2-(trifluoromethyl)nicotinate. Yield: 2.9 mg (4%).

### Method E

### Exemplified by the Procedure from Example 75

DIPEA (38 mg, 0.3 mmol) was added to a solution of 1-[3-cyano-6-(difluoromethyl)-5-(ethoxycarbonyl)pyridin-2-yl]piperidine-4-carboxylic (35.3 mg, 0.1 mmol) and TBTU (38.5 mg, 0.12 mmol) in DCM (2 mL) and the mixture was stirred for 10 min at r.t before 1-(2-fluorophenyl)methanesulfonamide (19 mg, 0.10 mmol) was added. The reaction was allowed to stir over night. The reaction mixture was washed with 1M KHSO₄ and the organic phase was passed through a phase separator and evaporated in a vacuum centrifuge. The crude product obtained was purified by HPLC (See General Experimental Procedure) to give ethyl 5-cyano-6-[4-({[(2-fluorobenzyl)sulfonyl]amino}carbonyl)piperidin-1-yl]-2-(fluoromethyl)nicotinate. Yield: 13 mg (25%).

### Example 1

### Ethyl 6-(4-{[(benzylsulfonyl)amino]carbonyl}piperidin-1-yl)-5-chloro-2-(difluoromethyl)nicotinate

### (a) Ethyl 2-(difluoromethyl)-6-oxo-1,6-dihydropyridine-3-carboxylate

Ethyl 2-methyl-6-oxo-1,6-dihydropyridine-3-carboxylate (2.0 g, 11.04 mmol) (Sobczak, A et al, Synth. Commun, Vol. 35, No. 23, 2005, pp 2993-3001) was added to a solution of 2-methoxy-N-(2-methoxyethyl)-N-(trifluoro-λ⁴-sulfanyl)ethanamine (7.82 g, 22.08 mmol) in CH₃CN under an atmosphere of nitrogen. The reaction was refluxed over night after which further 2-methoxy-N-(2-methoxyethyl)-N-(trifluoro-λ⁴-sulfanyl)ethanamine (2.73 g, 7.7 mmol) was added and the stirring was continued until all starting material was consumed. The reaction was diluted with diethyl ether, filtered to remove black solids, washed with water and NaHCO₃ (aq,sat). Both phases were filtered again to remove more of black solids. The aqueous phase was extracted with diethyl ether (2 times) and the combined organic phase was dried (MgSO₄), filtered and concentrated and slurried in diethyl ether to remove yellow impurities. Drying of the remaining white solid gave ethyl 2-(difluoromethyl)-6-oxo-1,6-dihydropyridine-3-carboxylate. Yield: 370 mg (14%).

¹H NMR (400 MHz, CDCl₃) δ 1.38 (3H, t, J=7.2 Hz), 4.36 (2H, q, J=7.2 Hz), 6.69 (1H, d, J=10 Hz), 7.56 (1H, t, J=54 Hz), 7.99 (1H, d, J=10 Hz).

### (b) Ethyl 5-chloro-2-(difluoromethyl)-6-oxo-1,6-dihydropyridine-3-carboxylate

NCS (270 mg, 2.02 mmol) dissolved in DMF (2 ml) was added to a solution of ethyl 2-(difluoromethyl)-6-oxo-1,6-dihydropyridine-3-carboxylate (365 mg, 1.44 mmol) and the reaction was heated to 100° C. over night. since staring material still remained further aliquots of NCS (135 mg, 1.01 mmol and 5 hours later 270 mg, 2.02 mmol) was added and the heating was continued until the starting material had disappeared. The reaction was diluted with DCM and washed with water and brine. The water phase was extracted twice with DCM and the combined organic phase was passed through a phase separator and evaporated. Purification by flash chromatography (horizon flash 40+M, Eluent: a gradient of EtOAc/heptane from 50 to 100% EtOAc was used)) gave ethyl 5-chloro-2-(difluoromethyl)-6-oxo-1,6-dihydropyridine-3-carboxylate as a yellow oil which was used in the next step without further analysis or purification. Yield: 88 mg (15%).

### (c) Ethyl 5,6-dichloro-2-(difluoromethyl)nicotinate

Oxalylchloride (0.1 ml, 1.18 mmol) together with DMF (0.1 ml) was added to a solution of ethyl 5-chloro-2-(difluoromethyl)-6-oxo-1,6-dihydropyridine-3-carboxylate (85.5 mg, 0.217 mmol) in DCM and the mixture was heated to 42° C. for 3 hours. No product could be detected and therefore another 0.1 ml (1.18 mmol) oxalylchloride was added and the stirring was continued at 42° C. over night. The reaction was diluted with DCM and quenched by pouring it on an ice/water mixture. The phases was separated and the organic phase was washed with NaHCO₃ (aq, sat) and brine. The combined water phase was extracted with DCM and the combined organic phase was filtered through a phase separator and evaporated. The residue was co-concentrated twice with DCM to give ethyl 5,6-dichloro-2-(difluoromethyl)nicotinate as a yellow oil which was used in the next step without further purification. Yield: 113 mg (51%).

### (d) tert-Butyl 4-[(benzylsulfonyl)carbamoyl]piperidine-1-carboxylate

Triethylamine (591 g, 5840 mmol) was added to a stirred suspension of 1-(tert-butoxycarbonyl)piperidine-4-carboxylic acid (448 g, 1954 mmol), LiCl (23.1 g, 545 mmol) and TBTU (657 g, 2046 mmol) in THF (3000 mL) under an atmosphere of nitrogen at r.t. A solution of 1-phenylmethanesulfonamide (352 g in 1300 mL THF, 2056 mmol) was added after 1.5 hours and the stirring was continued over night. The solvent was removed in vacuo to give a thick grey-beige slurry (volume about 2500 mL). EtOAc (3500 mL) was added followed by an aqueous solution of HCl (1960 mL 3.6 M HCl and 1960 mL water). The water phase was removed and the organic phase was washed with 2×1500 mL 1 M HCl. The organic phase was cooled to 0° C. which gave a precipitate of HOBT that was filtered off. Most of the solvent was removed in vacuo to give a thick grey-white slurry. EtOH (50%, 4000 mL) was added and the slurry was stirred for 1.5 hours. The precipitated product was filtered off, washed with 50% EtOH (500 mL+2×1500 mL) and dried in a vacuum oven at 25° C. to give tert-butyl 4-[(benzylsulfonyl)carbamoyl]piperidine-1-carboxylate as a white solid. Yield: 584 g (78%).

### (e) N-(benzylsulfonyl)piperidine-4-carboxamide

tert-Butyl 4-[(benzylsulfonyl)carbamoyl]piperidine-1-carboxylate (583 g, 1524 mmol) was suspended in formic acid (3000 mL) under a nitrogen atmosphere and the reaction was stirred for 20 minutes. The reaction was foaming due to the gas evolution and formic acid (500 mL) was used to wash down the foam from the reaction vessel walls. After 2 hours the foaming had stopped and the reaction was clear with a few solids left. The reaction was stirred over night and 2500 mL of formic acid was removed in vacuo. Water (1000 mL) was added and the reaction was filtered. The clear solution was evaporated and water (3000 mL) was added. A saturated ammonium hydroxide solution in water was used (totally 390 mL was added and the pH was going from 3.10 to 6.10) to neutralize the acidic solution and at the endpoint (pH=6.10) a heavy precipitate of the product was formed. The mixture was stirred over night and the precipitate was filtered off and washed with water (1000 mL). Drying in a vacuum oven at 25° C. gave N-(benzylsulfonyl)piperidine-4-carboxamide as a white powder. Yield: 372.4 g (87%).

### (f) Ethyl 6-(4-{[(benzylsulfonyl)amino]carbonyl}piperidin-1-yl)-5-chloro-2-(difluoromethyl)nicotinate

TEA (149 μL, 1.07 mmol) was added to a solution of ethyl 5,6-dichloro-2-(difluoromethyl)nicotinate (113 mg, 0.214 mmol)) and N-(benzylsulfonyl)piperidine-4-carboxamide (66 mg, 0.24 mmol) in CH₃CN (3 mL) and water (2 mL). The reaction was heated in a single node microwave oven at 120° C. over 20 minutes. The solvents were removed in vacuo and the crude mixture was diluted with DCM and washed twice with 1% KHSO₄(aq). The combined aqueous phase was extracted with DCM and the combined organic phases were passed through a phase separator followed by removal of solvents in vacuo. The crude product was purified using preparative HPLC on a (Kromasil C₈, 10 μm, 50.8×300 mm), the compound was loaded onto the column using 5% acetonitrile/aqueous NH₄OAc buffer pH 7 and then eluted using a gradient of 30-100% acetonitrile/aqueous NH₄OAc buffer pH 3.

Product-fractions were combined and the solvent was removed in vacuo, and triturated with DCM followed by filtration. The solvents were removed in vacuo to give ethyl 6-(4-{[(benzylsulfonyl)amino]carbonyl}piperidin-1-yl)-5-chloro-2-(difluoromethyl)nicotinate as a white solid. Yield: 13 mg (11%).

¹H NMR (400 MHz, CDCl₃) [symbol] 1.38 (3H, t, J=7.1 Hz), 1.73-1.91 (4H, m), 2.27-2.42 (1H, m), 2.87-3.05 (2H, m), 4.19-4.30 (2H, m), 4.30-4.41 (2H, m), 4.67 (2H, s), 7.29-7.43 (5H, m), 7.48-7.54 (1H, m), 8.16 (1H, s)

### Example 2

### Ethyl 6-(4-{[(benzylsulfonyl)amino]carbonyl}piperidin-1-yl)-5-cyano-2-(difluoromethyl)nicotinate

### (a) Ethyl 5-cyano-2-(difluoromethyl)-6-oxo-1,6-dihydropyridine-3-carboxylate

1,1-Dimethoxy-N,N-dimethylmethanamine (4.8 mL, 36.1 mmol) was added to ethyl 4,4-difluoro-3-oxobutanoate (5.0 g, 30.1 mmol) (exothermic reaction). The orange solution was stirred at r.t over night, concentrated and co-evaporated with toluene. The residue was taken up in EtOH (99.5%, 10 mL) to give a red solution. Freshly prepared NaOEt (1M, 30 mL) was added to a solution of 2-cyanoacetamide (2.53 g, 30.1 mmol) in EtOH (99.5%, 30 mL) and the reaction was stirred at r.t for 1 hour and the above red solution was added dropwise. The red suspension formed was stirred over night and AcOH (6 mL) was added and the solution became clear. The solution was concentrated and slurried in water (50 mL) and stirred for 1 hour after which the precipitate was filtered off and dried in air to give ethyl 5-cyano-2-(difluoromethyl)-6-oxo-1,6-dihydropyridine-3-carboxylate as a brown solid. Yield: 3.03 g (41%).

¹H-NMR (400 MHz, DMSO-d₆) δ 1.30 (3H, t, J=7.2 Hz), 4.28 (2H, q, J=7.2 Hz), 7.48 (1H, t, J=52.5 Hz, F-coupling), 8.58 (1H, s).

### (b) Ethyl 6-chloro-5-cyano-2-(difluoromethyl)nicotinate

Oxalylchloride (5.3 mL, 62.6 mmol) followed by DMF (0.097 mL) was added to a slurry of ethyl 5-cyano-2-(difluoromethyl)-6-oxo-1,6-dihydropyridine-3-carboxylate (3.0 g, 12.5 mmol) in DCM (45 mL) and the reaction was heated to 50° C. for a few hours, more oxalylchloride was added (1 mL, 11.8 mmol) and DMF (0.2 mL) was added twice with a few hours in between and the heating was continued at reflux over night. The reaction mixture was evaporated and the residue was taken up in DCM and washed with water and NaHCO₃ (aq,sat). The aqueous phase was extracted with DCM (twice) and the combined organic phase was concentrated and purified by flash chromatography (Horizon, Eluent a gradient of Heptane/EtOAc 7/1 to 100% EtOAc was used) to give ethyl 6-chloro-5-cyano-2-(difluoromethyl)nicotinate as a yellow oil. Yield: 2.0 g (60%).

¹H-NMR (400 MHz, DMSO-d₆) δ 1.34 (3H, t, J=7.0), 4.37 (2H, q, J=7.0 Hz), 7.46 (1H, t, J=53.2 Hz), 8.99 (1H, s).

### (c) Ethyl 6-(4-{[(benzylsulfonyl)amino]carbonyl}piperidin-1-yl)-5-cyano-2-(difluoromethyl)nicotinate

TEA (0.4 mL, 2.89 mmol) was added to a solution of ethyl 6-chloro-5-cyano-2-(difluoromethyl)nicotinate (200 mg, 0.721 mmol) and N-(benzylsulfonyl)piperidine-4-carboxamide (224 mg, 0.793 mmol) in water (2.5 mL) and EtOH (2 mL). The mixture was heated in a single-node microwave oven at 120° C. for 20 minutes, The solvents were evaporated and the residue was taken up in DCM and washed with 1% KHSO₄ (twice). The combined aqueous phase was extracted with DCM (twice) and the combined organic phase was filtered through a phase separator and concentrated. Purification by HPLC (Kromasil C₈, 10 μm, Eluent: A gradient of 40% CH₃CN to 100% CH₃CN/(50 mM HCOOH and 50 mM NH₄OOCH, pH=3) gave ethyl 6-(4-{[(benzylsulfonyl)amino]carbonyl}piperidin-1-yl)-5-cyano-2-(difluoromethyl)nicotinate as a white solid. Yield: 250 mg (68%).

¹H NMR (400 MHz, DMSO-d₆) δ 1.31 (3H, t, J=7.4 Hz), 1.73-1.59 (2H, m), 1.91-1.81 (2H, m), 2.61 (1H, m), 3.27-3.15 (2H, m), 4.28 (2H, q, J=7.4 Hz), 4.61-4.51 (2H, m), 4.69 (2H, s), 7.33-7.22 (2H, m), 7.44-7.34 (3H, m), 7.53 (1H, s), 8.50 (1H, s), 11.61 (1H, s)

### Example 3

### Ethyl 6-(4-{[(benzylsulfonyl)amino]carbonyl}piperidin-1-yl)-5-cyano-2-(trifluoromethyl)nicotinate

### (a) Ethyl 6-chloro-5-cyano-2-(trifluoromethyl)nicotinate

Oxalylchloride (12.20 g, 96.1 mmol) and DMF (0.744 mL) were added to a solution of ethyl 5-cyano-6-oxo-2-(trifluoromethyl)-1,6-dihydropyridine-3-carboxylate (5 g, 19.22 mmol) (prepared essentially according to the method described in Mosti, L et al, Farmaco, Vol 47, No 4, 1992, pp. 427-437) and the reaction was heated to 50° C. over night. The reaction was evaporated and the crude was dissolved in EtOAc and water. The phases was separated and the organic phase was washed with Brine and NaHCO₃ (aq,sat). The aqueous phase was extracted with EtOAc (3 times) and the combined organic phase was dried (Na₂CO₃), filtered and concentrated to give ethyl 6-chloro-5-cyano-2-(trifluoromethyl)nicotinate as a brown solid which was used without further purification. Yield: 5.21 g (95%).

¹H NMR (400 MHz, DMSO-d₆) δ 1.31 (3H, t, J=7.2 Hz), 4.38 (2H, q, J=6.9 Hz), 9.07 (1H, s)

### (b) Ethyl 6-(4-{[(benzylsulfonyl)amino]carbonyl}piperidin-1-yl)-5-cyano-2-(trifluoromethyl)nicotinate

TEA (142 mg, 1.41 mmol) was added to a solution of ethyl 6-chloro-5-cyano-2-(trifluoromethyl)nicotinate (140 mg, 0.352 mmol) and N-(benzylsulfonyl)piperidine-4-carboxamide (109 mg, 0.387 mmol) in water (2 ml) and EtOH (2.5 ml). The mixture was heated in a single-node microwave oven at 120° C. for 20 minutes, the solvents were evaporated and the residue was taken up in DCM and washed with 1% KHSO₄ (twice). The combined aqueous phase was extracted with DCM (twice) and the combined organic phase was filtered through a phase separator and concentrated. Purification by HPLC (Kromasil C₈, 10 μm, Eluent: a gradient of 30% CH₃CN to 100% CH₃CN/(50 mm HCOOH and 50 mm NH₄OOCH, pH=3) gave ethyl 6-(4-{[(benzylsulfonyl)amino]carbonyl}piperidin-1-yl)-5-cyano-2-(trifluoromethyl)nicotinate as a white solid. Yield: 107 mg (58%).

¹H NMR (400 mhz, DMSO-d₆) δ 1.29 (3H, t, J=7.5 Hz), 1.74-1.58 (2H, m), 1.91-1.79 (2H, m), 2.65-2.54 (1H, m), 3.27-3.15 (2H, m), 4.28 (2H, q, J=7.5 Hz), 4.55-4.46 (2H, m), 4.68 (2H, s), 7.33-7.23 (2H, m), 7.47-7.35 (3H, m), 8.54 (1H, s), 11.61 (1H, s).

### Example 4

### Ethyl 6-(3-{[(benzylsulfonyl)amino]carbonyl}azetidin-1-yl)-5-cyano-2-(difluoromethyl)nicotinate

### (a) 1-(tert-Butoxycarbonyl)azetidine-3-carboxylic acid

(Boc)₂O (25.535 g, 117 mmol) dissolved in MeOH (70 mL) was added dropwise during 20 minutes to a stirred slurry of azetidine-3-carboxylic acid (10.11 g, 100 mmol) and Et₃N (27.8 mL, 200 mmol) in MeOH (105 mL) at r.t (mildly exothermic reaction) and the mixture was stirred over night (18 hours). The reaction was evaporated to dryness and THF (120 mL) was added and evaporated to give crude 1-(tert-butoxycarbonyl)azetidine-3-carboxylic acid which was used without further purification in the next step. Yield: 25.89 g (128%)

¹H NMR (400 MHz, CDCl₃) δ 1.43 (9H, s), 3.21-3.34 (1H, m), 4.00-4.13 (4H, m).

### (b) tert-Butyl 3-[(benzylsulfonyl)carbamoyl]azetidine-1-carboxylate

TBTU (33.71 g, 105 mmol) and TEA (30.3 g, 300 mmol) was added to a solution of 1-(tert-butoxycarbonyl)azetidine-3-carboxylic acid from above (25.89 g, assumed to contain 100 mmol) in THF (200 mL) and the reaction was stirred at r.t for 30 minutes. 1-phenylmethanesulfonamide (17.97 g, 105 mmol) and LiCl (1.844 g, 43.5 mmol) was added and the stirring was continued at r.t over night (23 hours). The reaction was concentrated to about ⅓ was left and EtOAc (500 mL) was added and the organic phase was washed with 2 M HCl (1×150 mL, 2×50 mL), water (2×50 mL). Drying (MgSO₄), filtration and evaporation of the solvent gave a brown powder (48.6 g). The powder was slurried in 150 mL TBME and stirred 3 hours. The solids was filtered off and washed with TBME (40 mL). This procedure was repeated twice with 100 mL TBME (washing with 25 mL) to give a brownish powder (33 g) still containing some HOBT. The powder was dissolved in about 100 mL warm EtOH and water (130 mL) was added to induce a crystallisation of the product. The crystals was filtered off and dried to give pure tert-butyl 3-[(benzylsulfonyl)carbamoyl]azetidine-1-carboxylate as an off white powder. Yield: 25.4 g (71%).

¹H NMR (400 MHz, DMSO-d₆) δ 1.39 (9H, s), 3.30 (1H, m, overlapping with the water signal in DMSO), 3.78-3.95 (4H, m), 4.73 (2H, s), 7.28-7.34 (2H, m), 7.36-7.41 (3H, m), 11.71 (1H, br s). MS m/z: 353 (M−1).

### (c) N-(benzylsulfonyl)azetidine-3-carboxamide

tert-Butyl 3-[(benzylsulfonyl)carbamoyl]azetidine-1-carboxylate (25.4 g, 71.7 mmol) was added to HCOOH (300 mL) at r.t and the reaction was stirred over night (22 hours). The formic acid was removed in vacuo, water (40 mL) was added and removed in vacuo. Water (130 mL) was added to the residue followed by NH₄OH (aq) until pH reached 7.4 when a crystallization started. The crystals was filtered off and dried to give pure N-(benzylsulfonyl)azetidine-3-carboxamide as a white solid. Yield: 15.73 g (86%).

¹H NMR (400 MHz, DMSO-d₆) δ 3.22 (1H, m), 3.87-3.96 (4H, m), 4.28 (2H, s), 7.20-7.32 (5H, m). MS m/z: 255 (M+1)

### (d) Ethyl 6-(3-{[(benzylsulfonyl)amino]carbonyl}azetidin-1-yl)-5-cyano-2-(difluoromethyl)nicotinate

TEA (291 mg, 2.88 mmol) was added to a solution of ethyl 6-chloro-5-cyano-2-(difluoromethyl)nicotinate (200 mg, 0.721 mmol) and N-(benzylsulfonyl)azetidine-3-carboxamide (201 mg, 0.793 mmol) in water (2 mL) and EtOH (2.5 mL). The mixture was heated in a single-node microwave oven at 120° C. for 20 minutes, The solvents were evaporated and the residue was taken up in DCM and washed with 1% KHSO₄ (twice). The combined aqueous phase was extracted with DCM (twice) and the combined organic phase was filtered through a phase separator and concentrated. Purification by HPLC (Kromasil C₈, 10 μm, Eluent: A gradient of 40% CH₃CN to 100% CH₃CN/(50 mM HCOOH and 50 mM NH₄OOCH, pH=3) gave ethyl 6-(3-{[(benzylsulfonyl)amino]carbonyl}azetidin-1-yl)-5-cyano-2-(difluoromethyl)nicotinate as a white solid. Yield: 264 mg (72%).

¹H NMR (400 MHz, DMSO-d₆) δ 1.30 (3H, t, J=7.3 Hz), 3.64-3.53 (1H, m), 4.27 (2H, q, J=6.9 Hz), 4.53-4.31 (4H, m), 4.75 (2H, s), 7.40-7.30 (5H, m), 7.40 (1H, t, J=53.6 Hz), 8.47 (1H, s), 11.81 (1H, s). MS m/z: 478 (M+1)

### Example 5

### Ethyl 6-(3-{[(benzylsulfonyl)amino]carbonyl}azetidin-1-yl)-5-cyano-2-(trifluoromethyl)nicotinate

### (a) Ethyl 6-chloro-5-cyano-2-(trifluoromethyl)nicotinate

Oxalylchloride (8.13 ml, 96.1 mmol) and DMF (0.744 ml, 9.61 mmol) were added to a solution of ethyl 5-cyano-6-oxo-2-(trifluoromethyl)-1,6-dihydropyridine-3-carboxylate (5.0 g, 19.22 mmol, prepared essentially according to the procedure described by Mosti L, et. al. Farmaco, Vol 47, No 4, 1992, pp. 427-437) and the reaction was heated to reflux over night. The solvent was evaporated and the residue was dissolved in EtOAc/water. The phases were separated and the organic phase was washed with brine and NaHCO₃ (aq) (twice). The aqueous phase was extracted with EtOAc (three times) and the combined organic phases was dried (Na₂CO₃), filtered and concentrated to give ethyl 6-chloro-5-cyano-2-(trifluoromethyl)nicotinate which was used without further purification. Yield: 5.21 g (95%).

¹H NMR (400 MHz, DMSO-d₆) δ 1.31 (3H, t, J=7 Hz), 4.38 (2H, q, J=7 Hz), 9.07 (1H, s).

### (b) Ethyl 6-(3-{[(benzylsulfonyl)amino]carbonyl}azetidin-1-yl)-5-cyano-2-(trifluoromethyl)nicotinate

TEA (142 mg, 1.41 mmol) was added to a solution of ethyl 6-chloro-5-cyano-2-(trifluoromethyl)nicotinate (140 mg, 0.352 mmol) and N-(benzylsulfonyl)azetidine-3-carboxamide (98.4 mg, 0.387 mmol) in water (2 mL) and EtOH (2.5 mL). The mixture was heated in a single-node microwave oven at 120° C. for 20 minutes. The reaction was filtered to remove a precipitate and the solvents were evaporated. The residue was taken up in DCM and washed with 1% KHSO₄ (twice). The combined aqueous phase was extracted with DCM (twice) and the combined organic phase was filtered through a phase separator and concentrated. Purification by HPLC (Kromasil C₈, 10 μm, Eluent: A gradient of 30% CH₃CN to 100% CH₃CN/(0.1% HCOOH(aq)) gave ethyl 6-(3-{[(benzylsulfonyl)amino]carbonyl}azetidin-1-yl)-5-cyano-2-(difluoromethyl)nicotinate as a white solid. Yield: 102 mg (58%).

¹H NMR (400 MHz, DMSO-d₆) δ 1.28 (3H, t, J=7.3 Hz), 3.63-3.52 (1H, m), 4.27 (2H, q, J=7.3 Hz), 4.52-4.31 (4H, m), 4.74 (2H, s), 8.50 (1H, s), 11.80 (1H, s). MS m/z: 496 (M+1)

### Example 6

### Ethyl 6-(4-{[(benzylsulfonyl)amino]carbonyl}piperidin-1-yl)-5-cyano-2-(fluoromethyl)nicotinate

### (a) Ethyl 5-cyano-2-(fluoromethyl)-6-oxo-1,6-dihydropyridine-3-carboxylate

1,1-dimethoxy-N,N-dimethylmethanamine (4.83 g, 40.5 mmol) was added to ethyl 4-fluoro-3-oxobutanoate (5.0 g, 33.75 mmol) at r.t (exothermic reaction) and the mixture was stirred over night, concentrated and co-evaporated with toluene. EtOH (99.5%, 10 mL) was added to give a red solution. Freshly prepared sodium ethoxide 1M solution (34.5 mL, 2.35 g, 34.5 mmol) was added to a solution of 2-cyanoacetamide (3.12 g, 37.13 mmol) in EtOH (99.5%, 30 mL) and after stirring at r.t for 35 minutes the red solution from above was added dropwise and the stirring continued over night. AcOH (6 mL) was carefully added (exothermic reaction) and the precipitate formed was filtered and washed with diethyl ester. Drying afforded ethyl 5-cyano-2-(fluoromethyl)-6-oxo-1,6-dihydropyridine-3-carboxylate as a beige solid. Yield: 4.42 g (56%).

¹H NMR (400 MHz, DMSO-d₆) δ 1.24 (3H, t, J=7.2 Hz), 4.12 (2H, q, J=6.9 Hz), 5.42 (2H, d, J=47.5 Hz), 7.96 (1H, s). MS m/z: 225 (M+1).

### (b) Ethyl 6-chloro-5-cyano-2-(fluoromethyl)nicotinate

Oxalylchloride (5.49 mL, 64.9 mmol) and DMF (0.5 mL, 6.5 mmol) were added to a solution of ethyl 5-cyano-2-(fluoromethyl)-6-oxo-1,6-dihydropyridine-3-carboxylate (3.0 g, 12.98 mmol) in DCM (120 mL) and the mixture was heated to reflux for 6 hours. The solvent was evaporated and the residue was dissolved in EtOAc/water. The phases were separated and the organic phase was washed with Brine and NaHCO₃ (aq). The aqueous phase was extracted with EtOAc (twice) and the combined organic phase was concentrated to give ethyl 6-chloro-5-cyano-2-(fluoromethyl)nicotinate as a beige solid which was used without further purification. Yield: 2.92 g (90%).

¹H NMR (400 MHz, DMSO-d₆) δ 1.33 (t, J=7.1 Hz, 3H), 4.34 (q, J=7.1 Hz, 2H), 5.88 (s, 1H), 5.77 (s, 1H), 8.89 (s, 1H). MS m/z: 243 (M+1)

### (c) Ethyl 6-(4-{[(benzylsulfonyl)amino]carbonyl}piperidin-1-yl)-5-cyano-2-(fluoromethyl)nicotinate

TEA (326 mg, 3.23 mmol) was added to a solution of ethyl 6-chloro-5-cyano-2-(fluoromethyl)nicotinate (200 mg, 0.81 mmol) and N-(benzylsulfonyl)piperidine-4-carboxamide (251 mg, 0.89 mmol) in CH₃CN (1.5 mL) and 95% EtOH (2.5 mL). The mixture was heated in a single-node microwave oven at 120° C. for 20 minutes. The solvent was evaporated and the residue was taken up in DCM and washed with 1% KHSO₄ (twice). The combined aqueous phase was extracted with DCM and the combined organic phase was filtered through a phase separator and concentrated. Purification by HPLC (Kromasil C₈, 10 μm, Eluent: A gradient of 40% CH₃CN to 100% CH₃CN/(0.1% HCOOH(aq)) gave ethyl 6-(4-{[(benzylsulfonyl)amino]carbonyl}piperidin-1-yl)-5-cyano-2-(fluoromethyl)nicotinate as a beige solid. Yield: 257 mg (65%).

¹H NMR (400 MHz, DMSO-d₆) δ 1.30 (3H, t, J=7.2 Hz), 1.71-1.56 (2H, m), 1.89-1.79 (2H, m), 2.65-2.54 (1H, m), 3.24-3.12 (2H, m), 4.25 (2H, q, J=7.2 Hz), 4.64-4.53 (2H, m), 4.68 (2H, s), 5.63 (1H, s), 5.75 (1H, s), 7.33-7.23 (2H, m), 7.44-7.34 (3H, m), 8.40 (1H, s), 11.60 (1H, s). MS m/z: 489 (M+1)

### Example 7

### Ethyl 6-(3-{[(benzylsulfonyl)amino]carbonyl}azetidin-1-yl)-5-cyano-2-(fluoromethyl)nicotinate

TEA (326 mg, 3.23 mmol) was added to a solution of ethyl 6-chloro-5-cyano-2-(fluoromethyl)nicotinate (200 mg, 0.81 mmol) and N-(benzylsulfonyl)azetidine-3-carboxamide (225 mg, 0.89 mmol) in CH₃CN (1.5 mL) and 95% EtOH (2.5 mL). The mixture was heated in a single-node microwave oven at 120° C. for 20 minutes. The solvent was evaporated and the residue was taken up in DCM and washed with 1% KHSO₄. The combined aqueous phase was extracted with DCM and the combined organic phase was filtered through a phase separator and concentrated. Purification by HPLC (Kromasil C₈, 10 μm, Eluent: A gradient of 40% CH₃CN to 100% CH₃CN/(0.1% HCOOH(aq)) gave ethyl 6-(3-{[(benzylsulfonyl)amino]carbonyl}azetidin-1-yl)-5-cyano-2-(fluoromethyl)nicotinate as a beige solid. Yield: 221 mg (59%).

¹H NMR (400 MHz, DMSO-d₆) [symbol] 1.29 (3H, t, J=7.2 Hz), 3.62-3.51 (1H, m), 4.24 (2H, q, J=7.2 Hz), 4.39-4.29 (2H, m), 4.51-4.39 (2H, m), 4.74 (2H, s), 5.61 (1H, s), 5.73 (1H, s), 7.42-7.29 (5H, m), 8.38 (1H, s), 11.81 (1H, s). MS m/z: 461 (M+1).

### Example 8

### Ethyl 5-cyano-2-(difluoromethyl)-6-{4-[({[(4-methylcyclohexyl)methyl]sulfonyl}amino)carbonyl]piperidin-1-yl}nicotinate

### (a) 1-[3-Cyano-6-(difluoromethyl)-5-(ethoxycarbonyl)pyridin-2-yl]piperidine-4-carboxylic acid

TEA (423 mg, 4.18 mmol) was added to a solution of ethyl 6-chloro-5-cyano-2-(difluoromethyl)nicotinate (290 mg, 1.05 mmol) and piperidine-4-carboxylic acid (148 mg, 1.15 mmol) in water/EtOH (4.5 mL). The mixture was heated in a single-node microwave oven at 120° C. for 10 minutes. The solvent was evaporated and the residue was taken up in DCM and washed with 1% KHSO₄. The combined aqueous phase was extracted with DCM (twice) and the combined organic phase was filtered through a phase separator and concentrated to give 1-[3-cyano-6-(difluoromethyl)-5-(ethoxycarbonyl)pyridin-2-yl]piperidine-4-carboxylic acid as a white solid which was used without further purification. Yield: 356 mg (94%).

¹H-NMR (400 MHz, CDCl₃) δ 1.39 (3H, t, J=7.2 Hz), 1.84-1.97 (2H, m), 2.08-2.17 (2H, m), 2.69-2.79 (1H, m), 3.37-3.47 (2H, m), 4.37 (2H, q, J=7.2 Hz), 4.61-4.70 (2H, m), 7.39 (1H, t, CHF₂), 8.43 (1H, s). MS m/z: 354 (M+1)

### (b) Ethyl 5-cyano-2-(difluoromethyl)-6-{4-[({[(4-methylcyclohexyl)methyl]sulfonyl}amino)carbonyl]piperidin-1-yl}nicotinate

DIPEA (64 mg, 0.5 mmol) was added to a solution of 1-[3-cyano-6-(difluoromethyl)-5-(ethoxycarbonyl)pyridin-2-yl]piperidine-4-carboxylic acid (35.3 mg, 0.1 mmol) and TBTU (38.5 mg, 0.12 mmol) in DCM (5 mL) and the mixture was stirred for 30 minutes at r.t before 1-(4-methylcyclohexyl)methanesulfonamide (23 mg, 0.12 mmol) dissolved in DCM (1 mL) was added. The reaction was allowed to stir over night. LC-MS showed that starting material was left so more TBTU (19 mg, 0.06 mmol) and DIPEA (26 mg, 0.2 mmol) were added to the mixture and the stirring was continued for another 2 h. The reaction mixture was washed with 1% KHSO₄, the aqueous phase was extracted with DCM (1 mL) and the combined organic phase was passed through a phase separator and evaporated in a vacuum centrifuge. The crude product obtained was purified by HPLC (Kromasil C₈, 10 μm, using a gradient of 20% to 100% CH₃CN/0.2% AcOH(aq)) to give ethyl 5-cyano-2-(difluoromethyl)-6-{4-[({[(4-methylcyclohexyl)methyl]sulfonyl}amino)carbonyl]piperidin-1-yl}nicotinate as a white solid. Yield: 22 mg (40%).

¹H NMR (400 MHz, CDCl₃) δ 8.61 (1H, s), 8.42 (1H, s), 7.36 (1H, t, J=54.3 Hz), 4.75 (2H, m), 4.35 (2H, q, J=7.3 Hz), 3.46 (1H, m), 3.38-3.22 (3H, m), 2.59 (1H, m), 2.30-2.18 (1H, m), 2.10-1.97 (2H, m), 1.96-1.79 (3H, m), 1.75-1.47 (6H, m), 1.37 (3H, t, J=7.2 Hz), 1.22-1.04 (2H, m), 0.92-0.83 (3H, m). MS m/z: 527 (M+1)

### Example 9

### Ethyl 5-cyano-2-(difluoromethyl)-6-[3-({[(2-fluorobenzyl)sulfonyl]amino}carbonyl)azetidin-1-yl]nicotinate

### (a) 1-[3-Cyano-6-(difluoromethyl)-5-(ethoxycarbonyl)pyridin-2-yl]azetidine-3-carboxylic acid

TEA (423 mg, 4.18 mmol) was added to a solution of ethyl 6-chloro-5-cyano-2-(difluoromethyl)nicotinate (290 mg, 1.05 mmol) and azetidine-3-carboxylic acid (116 mg, 1.15 mmol) in 95% EtOH (4.5 mL). The mixture was heated in a single-node microwave oven at 120° C. for 10 minutes. The solvent was evaporated and the residue was taken up in DCM and washed with 1% KHSO₄. The combined aqueous phase was extracted with DCM (twice) and the combined organic phase was filtered through a phase separator and concentrated to give 1-[3-cyano-6-(difluoromethyl)-5-(ethoxycarbonyl)pyridin-2-yl]azetidine-3-carboxylic acid as a white solid which was used without further purification. Yield: 359 mg (101%).

¹H-NMR (400 MHz, CDCl₃) δ 1.39 (3H, t, J=7.1 Hz), 3.62-3.72 (1H, m), 4.36 (2H, q, J=7.1 Hz), 4.63-4.75 (4H, m), 7.34 (1H, t, J=54.2 Hz, CHF₂), 8.36 (1H, s). MS m/z: 326 (M+1)

### (b) Ethyl 5-cyano-2-(difluoromethyl)-6-[3-({[(2-fluorobenzyl)sulfonyl]amino}carbonyl)azetidin-1-yl]nicotinate

DIPEA (64 mg, 0.5 mmol) was added to a solution of 1-[3-cyano-6-(difluoromethyl)-5-(ethoxycarbonyl)pyridin-2-yl]azetidine-3-carboxylic acid (32.5 mg, 0.1 mmol) and TBTU (38.5 mg, 0.12 mmol) in DCM (5 mL) and the mixture was stirred for 30 min at r.t before 1-(2-fluorophenyl)methanesulfonamide (23 mg, 0.12 mmol) dissolved in DCM (1 mL) was added. The reaction was allowed to stir over night. LC-MS showed that starting material was left so more TBTU (19 mg, 0.06 mmol) and DIPEA (26 mg, 0.2 mmol) were added to the mixture and the stirring was continued for another 2 h. The reaction mixture was washed with 1% KHSO₄, the aqueous phase was extracted with DCM (1 ml) and the combined organic phase was passed through a phase separator and evaporated in vacuum centrifuge. The crude product obtained was purified by HPLC (Kromasil C₈, 10 μm, using a gradient of 20% to 100% CH₃CN/0.2% AcOH(aq)) to give ethyl 5-cyano-2-(difluoromethyl)-6-[3-({[(2-fluorobenzyl)sulfonyl]amino}carbonyl)azetidin-1-yl]nicotinate as a white solid. Yield: 42 mg (83%).

¹H NMR (400 MHz, CDCl₃) [symbol] 1.38 (3H, t, J=7.1 Hz), 3.50-3.40 (1H, m), 4.35 (2H, q, J=7.2 Hz), 4.67-4.51 (4H, m), 4.72 (2H, s), 7.22-7.08 (2H, m), 7.46-7.34 (2H, m), 7.44 (1H, t, CHF₂), 8.35 (1H, s). MS m/z: 497 (M+1)

### Example 10

### Ethyl 5-cyano-2-(difluoromethyl)-6-[4-({[(2-fluorobenzyl)sulfonyl]amino}carbonyl)piperidin-1-yl]nicotinate

Prepared according to Method A from 1-[3-cyano-6-(difluoromethyl)-5-(ethoxycarbonyl)pyridin-2-yl]piperidine-4-carboxylic acid and 1-(2-fluorophenyl)methanesulfonamide to give ethyl 5-cyano-2-(difluoromethyl)-6-[4-({[(2-fluorobenzyl)sulfonyl]amino}carbonyl)piperidin-1-yl]nicotinate. Yield: 41 mg (78%).

¹H NMR (600 MHz, DMSO-d₆) δ 1.28 (3H, t, J=6.8 Hz), 1.60-1.68 (2H, m), 1.85-1.90 (2H, m), 2.57-2.64 (1H, m), 3.17-3.24 (2H, m), 4.25 (2H, q, J=7.0 Hz), 4.53-4.58 (2H, m), 4.72 (2H, s), 7.20-7.26 (2H, m), 7.35-7.45 (2H, m), 7.37 (1H, t, J=54.1 Hz), 8.47 (1H, s). MS m/z: 525 (M+1)

### Example 11

### Ethyl 5-cyano-2-(difluoromethyl)-6-[4-({[(3-fluorobenzyl)sulfonyl]amino}carbonyl)piperidin-1-yl]nicotinate

Prepared according to Method A from 1-[3-cyano-6-(difluoromethyl)-5-(ethoxycarbonyl)pyridin-2-yl]piperidine-4-carboxylic acid and 1-(3-fluorophenyl)methanesulfonamide to give Ethyl 5-cyano-2-(difluoromethyl)-6-[4-({[(3-fluorobenzyl)sulfonyl]amino}carbonyl)piperidin-1-yl]nicotinate. Yield: 21 mg (40%).

¹H NMR (600 MHz, DMSO-d₆) δ 8.45 (1H, s), 7.35 (1H, t, J=53.5 Hz), 7.38-7.43 (1H, m), 7.16-7.22 (1H, m), 7.05-7.11 (2H, m), 4.69 (2H, s), 4.48-4.55 (2H, m), 4.24 (2H, q, J=7.1 Hz), 3.14-3.21 (2H, m), 2.53-2.58 (1H, m), 1.78-1.84 (2H, m), 1.56-1.65 (2H, m), 1.27 (3H, t, J=7.1 Hz). MS m/z: 525 (M+1)

### Example 12

### Ethyl 5-cyano-2-(difluoromethyl)-6-[4-({[(4-fluorobenzyl)sulfonyl]amino}carbonyl)piperidin-1-yl]nicotinate

Prepared according to Method A from 1-[3-cyano-6-(difluoromethyl)-5-(ethoxycarbonyl)pyridin-2-yl]piperidine-4-carboxylic acid and 1-(4-fluorophenyl)methanesulfonamide to give Ethyl 5-cyano-2-(difluoromethyl)-6-[4-({[(4-fluorobenzyl)sulfonyl]amino}carbonyl)piperidin-1-yl]nicotinate. Yield: 19 mg (36%).

¹H NMR (600 MHz, DMSO-d₆) δ 1.28 (3H, t, J=7.2 Hz), 1.58-1.67 (2H, m), 1.81-1.87 (2H, m), 3.15-3.22 (2H, m), 4.26 (2H, q, J=7.1 Hz), 4.51-4.58 (2H, m), 4.66 (2H, s), 7.19-7.23 (2H, m), 7.28-7.32 (2H, m), 7.37 (1H, t, J=54.1 Hz), 8.47 (1H, s) Note! One H is hidden in the DMSO signal. MS m/z: 525 (M+1)

### Example 13

### Ethyl 6-[4-({[(2-chlorobenzyl)sulfonyl]amino}carbonyl)piperidin-1-yl]-5-cyano-2-(difluoromethyl)nicotinate

Prepared according to Method A from 1-[3-cyano-6-(difluoromethyl)-5-(ethoxycarbonyl)pyridin-2-yl]piperidine-4-carboxylic acid and 1-(2-chlorophenyl)methanesulfonamide to give Ethyl 6-[4-({[(2-chlorobenzyl)sulfonyl]amino}carbonyl)piperidin-1-yl]-5-cyano-2-(difluoromethyl)nicotinate. Yield: 36 mg (67%).

¹H NMR (600 MHz, DMSO-d₆) δ 1.28 (3H, t, J=7.2 Hz), 1.60-1.69 (2H, m), 1.86-1.92 (2H, m), 3.18-3.24 (2H, m), 4.25 (2H, q, J=7.0 Hz), 4.51-4.59 (2H, m), 4.81 (2H, s), 7.26-7.53 (5H, m), 8.47 (1H, s). Note! One H is hidden in the DMSO signal MS m/z: 541 (M+1)

### Example 14

### Ethyl 6-[4-({[(3-chlorobenzyl)sulfonyl]amino}carbonyl)piperidin-1-yl]-5-cyano-2-(difluoromethyl)nicotinate

Prepared according to Method A from 1-[3-cyano-6-(difluoromethyl)-5-(ethoxycarbonyl)pyridin-2-yl]piperidine-4-carboxylic acid and 1-(3-chlorophenyl)methanesulfonamide to give Ethyl 6-[4-({[(3-chlorobenzyl)sulfonyl]amino}carbonyl)piperidin-1-yl]-5-cyano-2-(difluoromethyl)nicotinate. Yield: 42 mg (78%).

¹H NMR (600 MHz, DMSO-d₆) δ 1.27 (3H, t, J=6.8 Hz), 1.57-1.65 (2H, m), 1.78-1.84 (2H, m), 2.53-2.59 (1H, m), 3.14-3.21 (2H, m), 4.24 (2H, q, J=6.9 Hz), 4.49-4.56 (2H, m), 4.68 (2H, s), 7.18-7.46 (5H, m), 8.46 (1H, s). MS m/z: 541 (M+1)

### Example 15

### Ethyl 6-[4-({[(4-chlorobenzyl)sulfonyl]amino}carbonyl)piperidin-1-yl]-5-cyano-2-(difluoromethyl)nicotinate

Prepared according to Method A from 1-[3-cyano-6-(difluoromethyl)-5-(ethoxycarbonyl)pyridin-2-yl]piperidine-4-carboxylic acid and 1-(4-chlorophenyl)methanesulfonamide to give Ethyl 6-[4-({[(4-chlorobenzyl)sulfonyl]amino}carbonyl)piperidin-1-yl]-5-cyano-2-(difluoromethyl)nicotinate. Yield: 33 mg (61%).

¹H NMR (400 MHz, DMSO-d₆) δ 1.31 (3H, t, J=7.2 Hz), 1.58-1.72 (2H, m), 1.82-1.92 (2H, m), 2.56-2.68 (1H, m), 3.16-3.26 (2H, m), 4.28 (2H, q, J=7.2 Hz), 4.52-4.61 (2H, m), 4.70 (2H, s), 7.28-7.35 (2H, m), 7.39 (1H, t, J=54.1 Hz), 7.44-7.51 (2H, m), 8.50 (1H, s), 11.64 (1H, s). MS m/z: 541 (M+1)

### Example 16

### Ethyl 5-cyano-2-(difluoromethyl)-6-[4-({[(3-methylbenzyl)sulfonyl]amino}carbonyl)piperidin-1-yl]nicotinate

Prepared according to Method A from 1-[3-cyano-6-(difluoromethyl)-5-(ethoxycarbonyl)pyridin-2-yl]piperidine-4-carboxylic acid and 1-(3-methylphenyl)methanesulfonamideto give ethyl 5-cyano-2-(difluoromethyl)-6-[4-({[(3-methylbenzyl)sulfonyl]amino}carbonyl)piperidin-1-yl]nicotinate. Yield: 17 mg (32%).

¹H NMR (400 MHz, DMSO-d₆) [symbol] 1.31 (3H, t, J=7.3 Hz), 1.59-1.73 (2H, m), 1.79-1.89 (2H, m), 2.29 (3H, s), 2.54-2.64 (1H, m), 3.16-3.26 (2H, m), 4.28 (2H, q, J=7.4 Hz), 4.53-4.61 (2H, m), 4.63 (2H, s), 7.04-7.10 (2H, m), 7.16-7.22 (1H, m), 7.24-7.31 (1H, m), 7.39 (1H, t, J=53.9 Hz), 8.49 (1H, s), 11.59 (1H, s). MS m/z: 521 (M+1)

### Example 17

### Ethyl 5-cyano-2-(difluoromethyl)-6-[4-({[(4-methylbenzyl)sulfonyl]amino}carbonyl)piperidin-1-yl]nicotinate

Prepared according to Method A from 1-[3-cyano-6-(difluoromethyl)-5-(ethoxycarbonyl)pyridin-2-yl]piperidine-4-carboxylic acid and 1-(4-methylphenyl)methanesulfonamideto give ethyl 5-cyano-2-(difluoromethyl)-6-[4-({[(4-methylbenzyl)sulfonyl]amino}carbonyl)piperidin-1-yl]nicotinate. Yield: 19 mg (36%).

¹H NMR (600 MHz, DMSO-d₆) δ 1.27 (3H, t, J=7.2 Hz), 1.57-1.65 (2H, m), 1.79-1.85 (2H, m), 2.26 (3H, s), 3.14-3.21 (2H, m), 4.24 (2H, q, J=7.3 Hz), 4.50-4.56 (2H, m), 4.58 (2H, s), 7.10-7.18 (4H, m), 7.36 (1H, t, J=53.4 Hz), 8.46 (1H, s). Note! One H is hidden in the DMSO signal. MS m/z: 521 (M+1)

### Example 18

### Ethyl 5-cyano-6-[4-({[(2,4-dichlorobenzyl)sulfonyl]amino}carbonyl)piperidin-1-yl]-2-(difluoromethyl)nicotinate

Prepared according to Method A from 1-[3-cyano-6-(difluoromethyl)-5-(ethoxycarbonyl)pyridin-2-yl]piperidine-4-carboxylic acid and 1-(2,4-dichlorophenyl)methanesulfonamide to give ethyl 5-cyano-6-[4-({[(2,4-dichlorobenzyl)sulfonyl]amino}carbonyl)piperidin-1-yl]-2-(difluoromethyl)nicotinate. Yield: 27 mg (47%).

¹H NMR (600 MHz, DMSO-d₆) δ 1.28 (3H, t, J=7.0 Hz), 1.59-1.68 (2H, m), 1.87-1.93 (2H, m), 2.54-2.60 (1H, m), 3.18-3.24 (2H, m), 4.26 (2H, q, J=6.8 Hz), 4.52-4.58 (2H, m), 4.81 (2H, s), 7.26-7.52 (3H, m), 7.69 (1H, s), 8.47 (1H, s). MS m/z: 575 (M+1)

### Example 19

### Ethyl 5-cyano-2-(difluoromethyl)-6-[3-({[(3-fluorobenzyl)sulfonyl]amino}carbonyl)azetidin-1-yl]nicotinate

Prepared according to Method A from 1-[3-cyano-6-(difluoromethyl)-5-(ethoxycarbonyl)pyridin-2-yl]azetidine-3-carboxylic acid and 1-(3-fluorophenyl)methanesulfonamide to give Ethyl 5-cyano-2-(difluoromethyl)-6-[3-({[(3-fluorobenzyl)sulfonyl]amino}carbonyl)azetidin-1-yl]nicotinate. Yield: 47 mg (95%).

¹H NMR (600 MHz, DMSO-d₆) δ 1.28 (3H, t, J=7.3 Hz), 3.51-3.59 (1H, m), 4.25 (2H, q, J=7.4 Hz), 4.26-4.51 (4H, m), 4.75 (2H, s), 7.12-7.22 (3H, m), 7.35-7.42 (1H, m), 7.37 (1H, t, J=53.2 Hz), 8.44 (1H, s). MS m/z: 497 (M+1)

### Example 20

### Ethyl 5-cyano-2-(difluoromethyl)-6-[3-({[(4-fluorobenzyl)sulfonyl]amino}carbonyl)azetidin-1-yl]nicotinate

Prepared according to Method A from 1-[3-cyano-6-(difluoromethyl)-5-(ethoxycarbonyl)pyridin-2-yl]azetidine-3-carboxylic acid and 1-(4-fluorophenyl)methanesulfonamide to give Ethyl 5-cyano-2-(difluoromethyl)-6-[3-({[(4-fluorobenzyl)sulfonyl]amino}carbonyl)azetidin-1-yl]nicotinate. Yield: 41 mg (83%).

¹H NMR (600 MHz, DMSO-d₆) δ 1.26 (3H, t, J=7.1 Hz), 3.49-3.57 (1H, m), 4.23 (2H, q, J=7.1 Hz), 4.26-4.50 (4H, m), 4.69 (2H, s), 7.12-7.19 (2H, m), 7.32-7.37 (2H, m), 7.36 (1H, t, J=54.2 Hz), 8.43 (1H, s). MS m/z: 497 (M+1)

### Example 21

### Ethyl 6-[3-({[(2-chlorobenzyl)sulfonyl]amino}carbonyl)azetidin-1-yl]-5-cyano-2-(difluoromethyl)nicotinate

Prepared according to Method A from 1-[3-cyano-6-(difluoromethyl)-5-(ethoxycarbonyl)pyridin-2-yl]azetidine-3-carboxylic acid and 1-(2-chlorophenyl)methanesulfonamide to give ethyl 6-[3-({[(2-chlorobenzyl)sulfonyl]amino}carbonyl)azetidin-1-yl]-5-cyano-2-(difluoromethyl)nicotinate. Yield: 42 mg (82%).

¹H NMR (400 MHz, DMSO-d₆) [symbol] 1.30 (3H, t, J=7.2 Hz), 3.58-3.68 (1H, m), 4.27 (2H, q, J=7.5 Hz), 4.36-4.57 (4H, m), 4.90 (2H, s), 7.35-7.46 (2H, m), 7.40 (1H, t, J=54.2 Hz), 7.47-7.56 (2H, m), 8.47 (1H, s), 12.03 (1H, s). MS m/z: 513 (M+1)

### Example 22

### Ethyl 6-[3-({[(3-chlorobenzyl)sulfonyl]amino}carbonyl)azetidin-1-yl]-5-cyano-2-(difluoromethyl)nicotinate

Prepared according to Method A from 1-[3-cyano-6-(difluoromethyl)-5-(ethoxycarbonyl)pyridin-2-yl]azetidine-3-carboxylic acid and 1-(3-chlorophenyl)methanesulfonamide to give ethyl 6-[3-({[(3-chlorobenzyl)sulfonyl]amino}carbonyl)azetidin-1-yl]-5-cyano-2-(difluoromethyl)nicotinate. Yield: 46 mg (90%).

¹H NMR (600 MHz, DMSO-d₆) δ 1.28 (3H, t, J=7.1 Hz), 3.51-3.59 (1H, m), 4.24 (2H, q, J=7.2 Hz), 4.25-4.54 (4H, m), 4.76 (2H, s), 7.26-7.30 (1H, m), 7.35-7.47 (4H, m), 8.44 (1H, s). MS m/z: 513 (M+1)

### Example 23

### Ethyl 6-[3-({[(4-chlorobenzyl)sulfonyl]amino}carbonyl)azetidin-1-yl]-5-cyano-2-(difluoromethyl)nicotinate

Prepared according to Method A from 1-[3-cyano-6-(difluoromethyl)-5-(ethoxycarbonyl)pyridin-2-yl]azetidine-3-carboxylic acid and 1-(4-chlorophenyl)methanesulfonamide to give ethyl 6-[3-({[(4-chlorobenzyl)sulfonyl]amino}carbonyl)azetidin-1-yl]-5-cyano-2-(difluoromethyl)nicotinate. Yield: 45 mg (88%).

¹H NMR (600 MHz, DMSO-d₆) δ 1.26 (3H, t, J=7.0 Hz), 3.50-3.57 (1H, m), 4.23 (2H, q, J=7.0 Hz), 4.27-4.50 (4H, m), 4.70 (2H, s), 7.30-7.34 (2H, m), 7.36 (1H, t, J=53.8 Hz), 7.38-7.43 (2H, m), 8.43 (1H, s). MS m/z: 513 (M+1)

### Example 24

### Ethyl 5-cyano-2-(difluoromethyl)-6-[3-({[(3-methylbenzyl)sulfonyl]amino}carbonyl)azetidin-1-yl]nicotinate

Prepared according to Method A from 1-[3-cyano-6-(difluoromethyl)-5-(ethoxycarbonyl)pyridin-2-yl]azetidine-3-carboxylic acid and 1-(3-methylphenyl)methanesulfonamide to give ethyl 5-cyano-2-(difluoromethyl)-6-[3-({[(3-methylbenzyl)sulfonyl]amino}carbonyl)azetidin-1-yl]nicotinate. Yield: 36 mg (73%).

¹H NMR (600 MHz, DMSO-d₆) δ 1.26 (3H, t, J=7.6 Hz), 2.22 (3H, s), 3.48-3.56 (1H, m), 4.23 (2H, q, J=7.0 Hz), 4.24-4.49 (4H, m), 4.64 (2H, s), 7.06-7.10 (2H, m), 7.12-7.16 (1H, m), 7.19-7.23 (1H, m), 7.36 (1H, t, J=54.9 Hz), 8.43 (1H, s). MS m/z: 493 (M+1)

### Example 25

### Ethyl 5-cyano-2-(difluoromethyl)-6-[3-({[(4-methylbenzyl)sulfonyl]amino}carbonyl)azetidin-1-yl]nicotinate

Prepared according to Method A from 1-[3-cyano-6-(difluoromethyl)-5-(ethoxycarbonyl)pyridin-2-yl]azetidine-3-carboxylic acid and 1-(4-ethylphenyl)methanesulfonamide to give ethyl 5-cyano-2-(difluoromethyl)-6-[3-({[(4-ethylbenzyl)sulfonyl]amino}carbonyl)azetidin-1-yl]nicotinate. Yield: 31 mg (63%).

¹H NMR (600 MHz, DMSO-d₆) δ 1.26 (3H, t, J=6.9 Hz), 2.24 (3H, s), 3.47-3.55 (1H, m), 4.23 (2H, q, J=6.9 Hz), 4.26-4.49 (4H, m), 4.63 (2H, s), 7.11-7.19 (4H, m), 7.36 (1H, t, J=53.8 Hz), 8.43 (1H, s). MS m/z: 493 (M+1)

### Example 26

### Ethyl 5-cyano-6-[3-({[(2,4-dichlorobenzyl)sulfonyl]amino}carbonyl)azetidin-1-yl]-2-(difluoromethyl)nicotinate

Prepared according to Method A from 1-[3-cyano-6-(difluoromethyl)-5-(ethoxycarbonyl)pyridin-2-yl]azetidine-3-carboxylic acid and 1-(2,4-dichlorophenyl)methanesulfonamide to give ethyl 5-cyano-6-[3-({[(2,4-dichlorobenzyl)sulfonyl]amino}carbonyl)azetidin-1-yl]-2-(difluoromethyl)nicotinate. Yield: 7 mg (12%).

¹H NMR (600 MHz, DMSO-d₆) δ 1.26 (3H, t, J=7.3 Hz), 3.44-3.55 (1H, m), 4.23 (2H, q, J=7.3 Hz), 4.29-4.52 (4H, m), 4.67-4.83 (2H, m), 7.35 (1H, t, J=54.3 Hz), 7.38-7.50 (2H, m), 7.57-7.64 (1H, m), 8.42 (1H, s). MS m/z: 547 (M+1)

### Example 27

### Ethyl 5-cyano-2-(difluoromethyl)-6-{3-[({[(4-methylcyclohexyl)methyl]sulfonyl}amino)carbonyl]azetidin-1-yl}nicotinate

Prepared according to Method A from 1-[3-cyano-6-(difluoromethyl)-5-(ethoxycarbonyl)pyridin-2-yl]azetidine-3-carboxylic acid and 1-(4-ethylcyclohexyl)methanesulfonamide to give ethyl 5-cyano-2-(difluoromethyl)-6-{3-[({[(4-ethylcyclohexyl)methyl]sulfonyl}amino)carbonyl]azetidin-1-yl}nicotinate. Yield: 27 mg (55%).

¹H NMR (400 MHz, DMSO-d₆) δ 0.80-0.95 (3H, m), 1.01-1.20 (2H, m), 1.30 (3H, t, J=7.0 Hz), 1.40-1.58 (5H, m), 1.60-1.88 (2H, m), 2.04-2.15 (1H, m), 3.40-3.45 (2H, m), 3.59-3.69 (1H, m), 4.26 (2H, q, J=7.4 Hz), 4.33-4.58 (4H, m), 7.38 (1H, t, J=54.3 Hz), 8.46 (1H, s), 11.93 (1H, s). MS m/z: 499 (M+1)

### Example 28

### Ethyl 5-cyano-6-[3-({[(3-cyanophenyl)sulfonyl]amino}carbonyl)azetidin-1-yl]-2-(difluoromethyl)nicotinate

Prepared according to Method A from 1-[3-cyano-6-(difluoromethyl)-5-(ethoxycarbonyl)pyridin-2-yl]azetidine-3-carboxylic acid and 3-cyanobenzenesulfonamide to give ethyl 5-cyano-6-[3-({[(3-cyanophenyl)sulfonyl]amino}carbonyl)azetidin-1-yl]-2-(difluoromethyl)nicotinate. Yield: 47 mg (64%).

¹H NMR (600 MHz, DMSO-d₆) δ 1.24 (3H, t, J=7.2 Hz), 3.51-3.59 (1H, m), 4.15-4.30 (4H, m), 4.32-4.46 (2H, m), 7.32 (1H, t, J=53.6 Hz), 7.76-7.81 (1H, m), 8.09-8.29 (3H, m), 8.38 (1H, s). MS m/z: 490 (M+1)

### Example 29

### Ethyl 5-cyano-6-[3-({[(4-cyanophenyl)sulfonyl]amino}carbonyl)azetidin-1-yl]-2-(difluoromethyl)nicotinate

Prepared according to Method A from 1-[3-cyano-6-(difluoromethyl)-5-(ethoxycarbonyl)pyridin-2-yl]azetidine-3-carboxylic acid and 4-cyanobenzenesulfonamide to give ethyl 5-cyano-6-[3-({[(4-cyanophenyl)sulfonyl]amino}carbonyl)azetidin-1-yl]-2-(difluoromethyl)nicotinate. Yield: 42 mg (57%).

¹H NMR (400 MHz, DMSO-d₆) δ 1.28 (3H, t, J=8.0 Hz), 3.54-3.65 (1H, m), 4.18-4.33 (2H, m), 4.25 (2H, q, J=7.2 Hz), 4.34-4.63 (2H, m), 7.36 (1H, t, J=53.1 Hz), 7.75-7.89 (1H, m), 8.03-8.12 (3H, m), 8.42 (1H, s). MS m/z: 490 (M+1)

### Example 30

### Ethyl 5-cyano-2-(difluoromethyl)-6-{3-[({[4-(trifluoromethoxy)phenyl]sulfonyl}amino)carbonyl]azetidin-1-yl}nicotinate

Prepared according to Method A from 1-[3-cyano-6-(difluoromethyl)-5-(ethoxycarbonyl)pyridin-2-yl]azetidine-3-carboxylic acid and 4-(trifluoromethoxy)benzenesulfonamide to give ethyl 5-cyano-2-(difluoromethyl)-6-{3-[({[4-(trifluoromethoxy)phenyl]sulfonyl}amino)carbonyl]azetidin-1-yl}nicotinate. Yield: 37 mg (45%).

¹H NMR (600 MHz, DMSO-d₆) δ 1.25 (3H, t, J=7.2 Hz), 3.51-3.58 (1H, m), 4.15-4.26 (2H, m), 4.21 (2H, q, J=7.0 Hz), 4.33-4.46 (2H, m), 7.32 (1H, t, J=54.1 Hz), 7.53-7.59 (2H, m), 7.99-8.05 (2H, m), 8.39 (1H, s). MS m/z: 549 (M+1)

### Example 31

### Ethyl 5-cyano-2-(difluoromethyl)-6-{3-[({[2-(trifluoromethoxy)phenyl]sulfonyl}amino)carbonyl]azetidin-1-yl}nicotinate

Prepared according to Method A from 1-[3-cyano-6-(difluoromethyl)-5-(ethoxycarbonyl)pyridin-2-yl]azetidine-3-carboxylic acid and 2-(trifluoromethoxy)benzenesulfonamide to give ethyl 5-cyano-2-(difluoromethyl)-6-{3-[({[2-(trifluoromethoxy)phenyl]sulfonyl}amino)carbonyl]azetidin-1-yl}nicotinate. Yield: 44 mg (53%).

¹H NMR (600 MHz, DMSO-d₆) δ 1.25 (3H, t, J=6.8 Hz), 3.50-3.61 (1H, m), 4.14-4.27 (2H, m), 4.21 (2H, q, J=7.0 Hz), 4.30-4.51 (2H, m), 7.32 (1H, t, J=54.0 Hz), 7.48-7.60 (2H, m), 7.71-7.83 (1H, m), 8.01-8.08 (1H, m), 8.39 (1H, s). MS m/z: 549 (M+1)

### Example 32

### Ethyl 5-cyano-6-[3-({[(2-cyanobenzyl)sulfonyl]amino}carbonyl)azetidin-1-yl]-2-(difluoromethyl)nicotinate

Prepared according to Method A from 1-[3-cyano-6-(difluoromethyl)-5-(ethoxycarbonyl)pyridin-2-yl]azetidine-3-carboxylic acid and 1-(2-cyanophenyl)methanesulfonamide to give ethyl 5-cyano-6-[3-({[(2-cyanobenzyl)sulfonyl]amino}carbonyl)azetidin-1-yl]-2-(difluoromethyl)nicotinate. Yield: 52 mg (69%).

¹H NMR (600 MHz, DMSO-d₆) δ 1.28 (3H, t, J=7.2 Hz), 3.57-3.65 (1H, m), 4.24 (2H, q, J=7.2 Hz), 4.31-4.56 (4H, m), 4.89 (2H, s), 7.37 (1H, t, J=54.2 Hz), 7.54-7.63 (2H, m), 7.70-7.75 (1H, m), 7.84-7.89 (1H, m), 8.44 (1H, s). MS m/z: 504 (M+1)

### Example 33

### Ethyl 5-cyano-2-(difluoromethyl)-6-(3-{[(2-naphthylsulfonyl)amino]carbonyl}azetidin-1-yl)nicotinate

Prepared according to Method A from 1-[3-cyano-6-(difluoromethyl)-5-(ethoxycarbonyl)pyridin-2-yl]azetidine-3-carboxylic acid and naphthalene-2-sulfonamide to give ethyl 5-cyano-2-(difluoromethyl)-6-(3-{[(2-naphthylsulfonyl)amino]carbonyl}azetidin-1-yl)nicotinate. Yield: 48 mg (62%).

¹H NMR (600 MHz, DMSO-d₆) δ 1.24 (3H, t, J=7.1 Hz), 3.51-3.59 (1H, m), 4.13-4.25 (2H, m), 4.20 (2H, q, J=7.0 Hz), 4.33-4.45 (2H, m), 7.30 (1H, t, J=54.4 Hz), 7.62-7.71 (2H, m), 7.84-7.88 (1H, m), 7.99-8.03 (1H, m), 8.07-8.13 (1H, m), 8.15-8.20 (1H, m), 8.36 (1H, s), 8.54-8.59 (1H, m). MS m/z: 515 (M+1)

### Example 34

### Ethyl 6-(3-{[(butylsulfonyl)amino]carbonyl}azetidin-1-yl)-5-cyano-2-(difluoromethyl)nicotinate

Prepared according to Method A from 1-[3-cyano-6-(difluoromethyl)-5-(ethoxycarbonyl)pyridin-2-yl]azetidine-3-carboxylic acid and butane-1-sulfonamide to give ethyl 6-(3-{[(butylsulfonyl)amino]carbonyl}azetidin-1-yl)-5-cyano-2-(difluoromethyl)nicotinate. Yield: 44 mg (65%).

¹H NMR (600 MHz, DMSO-d₆) δ 0.85 (3H, t, J=7.1 Hz), 1.27 (3H, t, J=7.1 Hz), 1.36 (2H, sextet, J=7.2 Hz), 1.62 (2H, quintet, J=7.7 Hz), 3.36 (2H, t, J=7.8 Hz), 3.58-3.66 (1H, m), 4.23 (2H, q, J=6.6 Hz), 4.29-4.56 (4H, m), 7.36 (1H, t, J=54.8 Hz), 8.43 (1H, s). MS m/z: 445 (M+1)

### Example 35

### Ethyl 5-cyano-6-[4-({[(3-cyanophenyl)sulfonyl]amino}carbonyl)piperidin-1-yl]-2-(difluoromethyl)nicotinate

Prepared according to Method A from 1-[3-cyano-6-(difluoromethyl)-5-(ethoxycarbonyl)pyridin-2-yl]piperidine-4-carboxylic acid and 3-cyanobenzenesulfonamide to give ethyl 5-cyano-6-[4-({[(3-cyanophenyl)sulfonyl]amino}carbonyl)piperidin-1-yl]-2-(difluoromethyl)nicotinate. Yield: 9 mg (12%).

¹H NMR (600 MHz, DMSO-d₆) δ 1.27 (3H, t, J=6.9 Hz), 1.43-1.51 (2H, m), 1.79-1.85 (2H, m), 3.15-3.22 (2H, m), 4.24 (2H, q, J=7.3 Hz), 4.43-4.49 (2H, m), 7.34 (1H, t, J=54.2 Hz), 7.71-7.76 (1H, m), 8.02-8.08 (1H, m), 8.09-8.13 (1H, m), 8.17-8.21 (1H, m), 8.43 (1H, s). Note! One H signal is overlapping with the with the DMSO signal. MS m/z: 518 (M+1)

### Example 36

### Ethyl 5-cyano-6-[4-({[(4-cyanophenyl)sulfonyl]amino}carbonyl)piperidin-1-yl]-2-(difluoromethyl)nicotinate

Prepared according to Method A from 1-[3-cyano-6-(difluoromethyl)-5-(ethoxycarbonyl)pyridin-2-yl]piperidine-4-carboxylic acid and 4-cyanobenzenesulfonamide to give ethyl 5-cyano-6-[4-({[(4-cyanophenyl)sulfonyl]amino}carbonyl)piperidin-1-yl]-2-(difluoromethyl)nicotinate. Yield: 9 mg (12%).

¹H NMR (600 MHz, DMSO-d₆) δ 1.25 (3H, t, J=7.3 Hz), 1.41-1.49 (2H, m), 1.78-1.83 (2H, m), 3.15-3.21 (2H, m), 4.23 (2H, q, J=7.0 Hz), 4.41-4.46 (2H, m), 7.32 (1H, t, J=53.8 Hz), 7.92-8.01 (4H, m), 8.41 (1H, s). Note! One H signal is overlapping with the DMSO signal. MS m/z: 518 (M+1)

### Example 37

### Ethyl 5-cyano-2-(difluoromethyl)-6-{4-[({[4-(trifluoromethoxy)phenyl]sulfonyl}amino)carbonyl]piperidin-1-yl}nicotinate

Prepared according to Method A from 1-[3-cyano-6-(difluoromethyl)-5-(ethoxycarbonyl)pyridin-2-yl]piperidine-4-carboxylic acid and 4-(trifluoromethoxy)benzenesulfonamide to give ethyl 5-cyano-2-(difluoromethyl)-6-{4-[({[4-(trifluoromethoxy)phenyl]sulfonyl}amino)carbonyl]piperidin-1-yl}nicotinate. Yield: 17 mg (19%).

¹H NMR (600 MHz, DMSO-d₆) δ 1.25 (3H, t, J=7.6 Hz), 1.41-1.50 (2H, m), 1.79-1.84 (2H, m), 3.14-3.20 (2H, m), 4.23 (2H, q, J=7.2 Hz), 4.42-4.48 (2H, m), 7.32 (1H, t, J=54.6 Hz), 7.52-7.56 (2H, m), 7.95-8.00 (2H, m), 8.42 (1H, s). Note! One H signal is overlapping with the DMSO signal. MS m/z: 577 (M+1)

### Example 38

### Ethyl 5-cyano-2-(difluoromethyl)-6-{4-[({[2-(trifluoromethoxy)phenyl]sulfonyl}amino)carbonyl]piperidin-1-yl}nicotinate

Prepared according to Method A from 1-[3-cyano-6-(difluoromethyl)-5-(ethoxycarbonyl)pyridin-2-yl]piperidine-4-carboxylic acid and 2-(trifluoromethoxy)benzenesulfonamide to give ethyl 5-cyano-2-(difluoromethyl)-6-{4-[({[2-(trifluoromethoxy)phenyl]sulfonyl}amino)carbonyl]piperidin-1-yl}nicotinate. Yield: 50 mg (58%).

¹H NMR (400 MHz, DMSO-d₆) δ 1.29 (3H, t, J=6.9 Hz), 1.43-1.56 (2H, m), 1.81-1.90 (2H, m), 2.61-2.71 (1H, m), 3.16-3.28 (2H, m), 4.26 (2H, q, J=7.3 Hz), 4.46-4.54 (2H, m), 7.36 (1H, t, J=53.1 Hz), 7.53-7.61 (2H, m), 7.77-7.84 (1H, m), 8.00-8.06 (1H, m), 8.46 (1H, s). MS m/z: 577 (M+1)

### Example 39

### Ethyl 5-cyano-6-[4-({[(2-cyanobenzyl)sulfonyl]amino}carbonyl)piperidin-1-yl]-2-(difluoromethyl)nicotinate

Prepared according to Method A from 1-[3-cyano-6-(difluoromethyl)-5-(ethoxycarbonyl)pyridin-2-yl]piperidine-4-carboxylic acid and 2-(trifluoromethoxy)benzenesulfonamide to give ethyl 5-cyano-2-(difluoromethyl)-6-{4-[({[2-(trifluoromethoxy)phenyl]sulfonyl}amino)carbonyl]piperidin-1-yl}nicotinate. Yield: 14 mg (17%).

¹H NMR (600 MHz, DMSO-d₆) δ 1.28 (3H, t, J=6.9 Hz), 1.60-1.68 (2H, m), 1.87-1.93 (2H, m), 3.19-3.24 (2H, m), 4.25 (2H, q, J=6.8 Hz), 4.51-4.57 (2H, m), 4.81 (2H, s), 7.36 (1H, t, J=53.6 Hz), 7.49-7.52 (1H, m), 7.53-7.59 (1H, m), 7.70-7.75 (1H, m), 7.85-7.89 (1H, m), 8.47 (1H, s). Note! One H signal is overlapping with the DMSO signal. MS m/z: 532 (M+1).

### Example 40

### Ethyl 5-cyano-2-(difluoromethyl)-6-(4-{[(2-naphthylsulfonyl)amino]carbonyl}piperidin-1-yl)nicotinate

Prepared according to Method A from 1-[3-cyano-6-(difluoromethyl)-5-(ethoxycarbonyl)pyridin-2-yl]piperidine-4-carboxylic acid and naphthalene-2-sulfonamide to give ethyl 5-cyano-2-(difluoromethyl)-6-(4-{[(2-naphthylsulfonyl)amino]carbonyl}piperidin-1-yl)nicotinate. Yield: 31 mg (38%).

¹H NMR (600 MHz, DMSO-d₆) δ 1.24 (3H, t, J=7.1 Hz), 1.39-1.47 (2H, m), 1.78-1.83 (2H, m), 3.12-3.19 (2H, m), 4.22 (2H, q, J=7.1 Hz), 4.42-4.47 (2H, m), 7.31 (1H, t, J=53.5 Hz), 7.61-7.71 (2H, m), 7.79-7.84 (1H, m), 7.98-8.02 (1H, m), 8.07-8.10 (1H, m), 8.14-8.18 (1H, m), 8.40 (1H, s), 8.50-8.56 (1H, m). Note! One H signal is overlapping with the DMSO signal. MS m/z: 543 (M+1)

### Example 41

### Ethyl 6-(4-{[(butylsulfonyl)amino]carbonyl}piperidin-1-yl)-5-cyano-2-(difluoromethyl)nicotinate

Prepared according to Method A from 1-[3-cyano-6-(difluoromethyl)-5-(ethoxycarbonyl)pyridin-2-yl]piperidine-4-carboxylic acid and butane-1-sulfonamide to give ethyl 6-(4-{[(butylsulfonyl)amino]carbonyl}piperidin-1-yl)-5-cyano-2-(difluoromethyl)nicotinate. Yield: 36 mg (51%).

¹H NMR (400 MHz, DMSO-d₆) δ 0.86 (3H, t, J=7.2 Hz), 1.30 (3H, t, J=7.4 Hz), 1.33-1.43 (2H, m), 1.56-1.70 (4H, m), 1.90-1.98 (2H, m), 2.64-2.74 (1H, m), 3.20-3.29 (2H, m), 3.32-3.38 (2H, m), 4.28 (2H, q, J=7.3 Hz), 4.53-4.62 (2H, m), 7.38 (1H, t, J=53.8 Hz), 8.49 (1H, s), 11.71 (1H, s). MS m/z: 473 (M+1)

### Example 42

### Ethyl 6-(3-{2-[(benzylsulfonyl)amino]-2-oxoethyl}pyrrolidin-1-yl)-5-cyano-2-(trifluoromethyl)nicotinate

### (a) {1-[3-Cyano-5-(ethoxycarbonyl)-6-(trifluoromethyl)pyridin-2-yl]pyrrolidin-3-yl}acetic acid

TEA (606 mg, 5.99 mmol) was added to a solution of ethyl 6-chloro-5-cyano-2-(trifluoromethyl)nicotinate (341 mg, 1.2 mmol) and pyrrolidin-3-ylacetic acid (209 mg, 1.62 mmol) in water/EtOH (4.5 ml). The mixture was heated in a single-node microwave oven at 120° C. for 20 minutes. The solvent was evaporated and the residue was taken up in DCM and washed with 1% KHSO₄. The combined aqueous phase was extracted with DCM and the combined organic phase was filtered through a phase separator and concentrated. Purification by HPLC (Kromasil C₈, 10 μm, Eluent: a gradient of 5% CH₃CN to 100% CH₃CN/(0.2% AcOH(aq)) gave {1-[3-cyano-5-(ethoxycarbonyl)-6-(trifluoromethyl)pyridin-2-yl]pyrrolidin-3-yl}acetic acid as a white solid. Yield: 219 mg (49%).

¹H NMR (400 MHz, DMSO-d₆) δ 1.35 (3H, t, J=7.2 Hz), 1.85-1.68 (1H, m), 2.38-2.23 (1H, m), 2.64-2.47 (2H, m), 2.81-2.66 (1H, m), 3.57-3.40 (1H, m), 3.91-3.77 (1H, m), 4.08-3.97 (1H, m), 4.21-4.10 (1H, m), 4.33 (2H, q, J=7.3 Hz), 8.31 (1H, s). MS m/z: 371 (M+1)

### (b) Ethyl 6-(3-{2-[(benzylsulfonyl)amino]-2-oxoethyl}pyrrolidin-1-yl)-5-cyano-2-(trifluoromethyl)nicotinate

Prepared according to Method B from {1-[3-cyano-5-(ethoxycarbonyl)-6-(trifluoromethyl)pyridin-2-yl]pyrrolidin-3-yl}acetic acid and 1-phenylmethanesulfonamide to give ethyl 6-(3-{2-[(benzylsulfonyl)amino]-2-oxoethyl}pyrrolidin-1-yl)-5-cyano-2-(trifluoromethyl)nicotinate. Yield: 88 mg (84%).

¹H NMR (600 MHz, DMSO-d₆) δ 1.26 (3H, t, J=7.3 Hz), 1.59-1.68 (1H, m), 2.09-2.17 (1H, m), 2.40-2.44 (2H, m), 3.64-3.77 (1H, m), 3.81-3.91 (1H, m), 3.94-4.06 (1H, m), 4.24 (2H, q, J=7.0 Hz), 4.68 (2H, s), 7.24-7.39 (5H, m), 8.45 (1H, s). Note! One H hidden in the DMSO peak and one H hidden in the H₂O peak. MS m/z: 525 (M+1)

### Example 43

### Ethyl 5-cyano-6-[3-(2-oxo-2-{[(2-phenylethyl)sulfonyl]amino}ethyl)pyrrolidin-1-yl]-2-(trifluoromethyl)nicotinate

Prepared according to Method B from {1-[3-cyano-5-(ethoxycarbonyl)-6-(trifluoromethyl)pyridin-2-yl]pyrrolidin-3-yl}acetic acid and 2-phenylethanesulfonamideto give ethyl 5-cyano-6-[3-(2-oxo-2-{[(2-phenylethyl)sulfonyl]amino}ethyl)pyrrolidin-1-yl]-2-(trifluoromethyl)nicotinate. Yield: 73 mg (68%).

¹H NMR (600 MHz, DMSO-d₆) δ 1.25 (3H, t, J=7.0 Hz), 1.58-1.66 (1H, m), 2.05-2.13 (1H, m), 2.37-2.40 (2H, m), 2.92-2.98 (2H, m), 3.62-3.67 (2H, m), 3.67-3.75 (1H, m), 3.80-3.99 (2H, m), 4.23 (2H, q, J=7.3 Hz), 7.15-7.31 (5H, m), 8.43 (1H, s). Note! One H hidden in the DMSO peak and one H hidden in the H₂O peak. MS m/z: 537 (M−1)

### Example 44

### Ethyl 6-[3-(2-{[(5-chloro-2-thienyl)sulfonyl]amino}-2-oxoethyl)pyrrolidin-1-yl]-5-cyano-2-(trifluoromethyl)nicotinate

Prepared according to Method B from {1-[3-cyano-5-(ethoxycarbonyl)-6-(trifluoromethyl)pyridin-2-yl]pyrrolidin-3-yl}acetic acid and 5-chlorothiophene-2-sulfonamide to give ethyl 6-[3-(2-{[(5-chloro-2-thienyl)sulfonyl]amino}-2-oxoethyl)pyrrolidin-1-yl]-5-cyano-2-(trifluoromethyl)nicotinate. Yield: 86 mg (78%).

¹H NMR (500 MHz, DMSO-d₆) δ 1.29 (3H, t, J=6.9 Hz), 1.60-1.69 (1H, m), 2.06-2.14 (1H, m), 2.44-2.48 (1H, m), 2.55-2.60 (1H, m), 3.33-3.39 (1H, m), 3.68-3.76 (1H, m), 3.84-3.96 (2H, m), 4.28 (2H, q, J=7.2 Hz), 7.22 (1H, d, J=4.2 Hz), 7.63 (1H, d, J=4.2 Hz), 8.41 (1H, s). MS m/z: 549 (M−1)

### Example 45

### Ethyl 5-cyano-6-[3-({[(4-fluorobenzyl)sulfonyl]amino}carbonyl)azetidin-1-yl]-2-(trifluoromethyl)nicotinate

### (a) 1-[3-Cyano-5-(ethoxycarbonyl)-6-(trifluoromethyl)pyridin-2-yl]azetidine-3-carboxylic acid

TEA (0.908 g, 8.97 mmol) was added to a suspension of ethyl 6-chloro-5-cyano-2-(trifluoromethyl)nicotinate (1.0 g, 3.59 mmol) and azetidine-3-carboxylic acid (0.399 g, 3.95 mmol) in EtOH (10 mL) and the mixture was heated in a single-node microwave oven for 20 minutes. The solvent was evaporated and the residue was partioned between iPrOAc (10 mL)/water and Na₂CO₃. The aqueous phase was separated and made acidic by addition of concentrated HCl. The acidic water phase was extracted with iPrOAc (2×10 mL). The combined extracts was dried (MgSO₄) and evaporated to give 1-[3-cyano-5-(ethoxycarbonyl)-6-(trifluoromethyl)pyridin-2-yl]azetidine-3-carboxylic acid as a brown solid which was used without further purification. Yield: 1.04 g (84%).

¹H NMR (500 MHz, DMSO-d₆) δ 1.27 (3H, t, J=7.1 Hz), 3.55-3.62 (1H, m), 4.28 (2H, q, J=7.1 Hz), 4.38-4.58 (4H, m), 8.46 (1H, s).

### (b) Ethyl 5-cyano-6-[3-({[(4-fluorobenzyl)sulfonyl]amino}carbonyl)azetidin-1-yl]-2-(trifluoromethyl)nicotinate

Prepared according to Method D from 1-[3-cyano-5-(ethoxycarbonyl)-6-(trifluoromethyl)pyridin-2-yl]azetidine-3-carboxylic acid and 1-(4-fluorophenyl)methanesulfonamide to give ethyl 5-cyano-6-[3-({[(4-fluorobenzyl)sulfonyl]amino}carbonyl)azetidin-1-yl]-2-(trifluoromethyl)nicotinate. Yield: 2.9 mg (4%). MS m/z: 515 (M+1)

### Example 46

### Ethyl 5-cyano-6-[3-({[(3-fluorobenzyl)sulfonyl]amino}carbonyl)azetidin-1-yl]-2-(trifluoromethyl)nicotinate

Prepared according to Method D from 1-[3-cyano-5-(ethoxycarbonyl)-6-(trifluoromethyl)pyridin-2-yl]azetidine-3-carboxylic acid and 1-(3-fluorophenyl)methanesulfonamide to give ethyl 5-cyano-6-[3-({[(3-fluorobenzyl)sulfonyl]amino}carbonyl)azetidin-1-yl]-2-(trifluoromethyl)nicotinate. Yield: 46.2 mg (90%).

¹H NMR (500 MHz, DMSO-d₆) [symbol] 1.30 (3H, t, J=7.1 Hz), 3.46 (1H, quintet, J=7.4 Hz), 4.29 (2H, q, J=7.2 Hz), 4.44 (4H, br s), 4.58 (2H, s), 7.02-7.09 (3H, m), 7.29 (1H, td, J=8.0, 5.9 Hz), 8.18 (1H, s), 10.83 (1H, s). MS m/z: 515 (M+1)

### Example 47

### Ethyl 5-cyano-6-[3-({[(2-fluorobenzyl)sulfonyl]amino}carbonyl)azetidin-1-yl]-2-(trifluoromethyl)nicotinate

Prepared according to Method D from 1-[3-cyano-5-(ethoxycarbonyl)-6-(trifluoromethyl)pyridin-2-yl]azetidine-3-carboxylic acid and 1-(2-fluorophenyl)methanesulfonamide to give ethyl 5-cyano-6-[3-({[(2-fluorobenzyl)sulfonyl]amino}carbonyl)azetidin-1-yl]-2-(trifluoromethyl)nicotinate. Yield: 45.1 mg (88%).

¹H NMR (500 MHz, DMSO-d₆) δ 1.39 (3H, t, J=7.1 Hz), 3.60 (1H, tt, J=8.7, 6.0 Hz), 4.37 (2H, q, J=7.2 Hz), 4.52-4.67 (4H, m), 4.73 (2H, s), 7.15 (1H, t, J=9.0 Hz), 7.21 (1H, t, J=7.6 Hz), 7.42 (2H, dd, J=13.5, 7.1 Hz), 8.26 (1H, s), 10.65 (1H, s). MS m/z: 515 (M+1)

### Example 48

### Ethyl 5-cyano-6-[3-({[(4-methylbenzyl)sulfonyl]amino}carbonyl)azetidin-1-yl]-2-(trifluoromethyl)nicotinate

Prepared according to Method D from 1-[3-cyano-5-(ethoxycarbonyl)-6-(trifluoromethyl)pyridin-2-yl]azetidine-3-carboxylic acid and 1-(4-methylphenyl)methanesulfonamide to give ethyl 5-cyano-6-[3-({[(4-methylbenzyl)sulfonyl]amino}carbonyl)azetidin-1-yl]-2-(trifluoromethyl)nicotinate. Yield: 42.4 mg (55%).

¹H NMR (500 MHz, DMSO-d₆) δ 1.39 (3H, t, J=7.1 Hz), 2.37 (3H, s), 3.54 (1H, tt, J=8.3, 6.2 Hz), 4.37 (2H, q, J=7.1 Hz), 4.39-4.49 (4H, br s), 4.63 (2H, s), 7.20 (2H, d, J=7.8 Hz), 7.26 (2H, d, J=7.9 Hz), 8.27 (1H, s). MS m/z: 511 (M+1)

### Example 49

### Ethyl 5-cyano-6-[3-({[(3-methylbenzyl)sulfonyl]amino}carbonyl)azetidin-1-yl]-2-(trifluoromethyl)nicotinate

Prepared according to Method D from 1-[3-cyano-5-(ethoxycarbonyl)-6-(trifluoromethyl)pyridin-2-yl]azetidine-3-carboxylic acid and 1-(3-methylphenyl)methanesulfonamide to give ethyl 5-cyano-6-[3-({[(3-methylbenzyl)sulfonyl]amino}carbonyl)azetidin-1-yl]-2-(trifluoromethyl)nicotinate. MS m/z: 511 (M+1)

### Example 50

### Ethyl 6-[3-({[(4-chlorobenzyl)sulfonyl]amino}carbonyl)azetidin-1-yl]-5-cyano-2-(trifluoromethyl)nicotinate

Prepared according to Method D from 1-[3-cyano-5-(ethoxycarbonyl)-6-(trifluoromethyl)pyridin-2-yl]azetidine-3-carboxylic acid and 1-(4-chlorophenyl)methanesulfonamideto give ethyl 6-[3-({[(4-chlorobenzyl)sulfonyl]amino}carbonyl)azetidin-1-yl]-5-cyano-2-(trifluoromethyl)nicotinate. Yield: 0.96 mg (1%). MS m/z: 531 (M+1)

### Example 51

### Ethyl 6-[3-({[(2-chlorobenzyl)sulfonyl]amino}carbonyl)azetidin-1-yl]-5-cyano-2-(trifluoromethyl)nicotinate

Prepared according to Method D from 1-[3-cyano-5-(ethoxycarbonyl)-6-(trifluoromethyl)pyridin-2-yl]azetidine-3-carboxylic acid and 1-(2-chlorophenyl)methanesulfonamideto give ethyl 6-[3-({[(2-chlorobenzyl)sulfonyl]amino}carbonyl)azetidin-1-yl]-5-cyano-2-(trifluoromethyl)nicotinate. Yield: 49.9 mg (63%).

¹H NMR (500 MHz, DMSO-d₆) δ 1.38 (3H, t, J=7.1 Hz), 3.62 (1H, tt, J=8.8, 6.2 Hz), 4.37 (2H, q, J=7.2 Hz), 4.87 (2H, s), 7.35 (2H, quintet, J=7.6, 1.7 Hz), 7.48 (2H, ddd, J=13.5, 7.5, 1.7 Hz), 8.26 (1H, s), 10.98 (1H, s). MS m/z: 531 (M+1)

### Example 52

### Ethyl 6-[3-({[(3-chlorobenzyl)sulfonyl]amino}carbonyl)azetidin-1-yl]-5-cyano-2-(trifluoromethyl)nicotinate

Prepared according to Method D from 1-[3-cyano-5-(ethoxycarbonyl)-6-(trifluoromethyl)pyridin-2-yl]azetidine-3-carboxylic acid and 1-(3-chlorophenyl)methanesulfonamideto give ethyl 6-[3-({[(3-chlorobenzyl)sulfonyl]amino}carbonyl)azetidin-1-yl]-5-cyano-2-(trifluoromethyl)nicotinate. Yield: 21.6 mg (27%).

¹H NMR (500 MHz, DMSO-d₆) δ 1.39 (3H, t, J=7.1 Hz), 3.55 (1H, quintet, J=7.4 Hz), 4.37 (2H, q, J=7.1 Hz), 4.49-4.57 (4H, m), 4.65 (2H, s), 7.26 (1H, d, J=7.7 Hz), 7.35 (1H, t, J=7.9 Hz), 7.41 (1H, d, J=8.0 Hz), 7.41 (1H, s), 8.27 (1H, s), 10.78 (1H, s). MS m/z: 531 (M+1)

### Example 53

### Ethyl 5-cyano-6-[3-({[(2,4-dichlorobenzyl)sulfonyl]amino}carbonyl)azetidin-1-yl]-2-(trifluoromethyl)nicotinate

Prepared according to Method D from 1-[3-cyano-5-(ethoxycarbonyl)-6-(trifluoromethyl)pyridin-2-yl]azetidine-3-carboxylic acid and 1-(2,4-dichlorophenyl)methanesulfonamide to give Ethyl 5-cyano-6-[3-({[(2,4-dichlorobenzyl)sulfonyl]amino}carbonyl)azetidin-1-yl]-2-(trifluoromethyl)nicotinate. Yield: 14.1 mg (16%).

¹H NMR (500 MHz, DMSO-d₆) δ 1.39 (3H, t, J=7.1 Hz), 3.64 (1H, tt, J=8.7, 6.0 Hz), 4.37 (2H, q, J=7.1 Hz), 4.52-4.70 (4H, br s), 4.84 (2H, s), 7.33 (1H, dd, J=8.4, 2.0 Hz), 7.45 (1H, d, J=8.3 Hz), 7.50 (1H, d, J=2.0 Hz), 8.27 (1H, s), 11.41 (1H, s). MS m/z: 565 (M+1)

### Example 54

### Ethyl 6-(3-{[(5-chloro-2-thienyl)sulfonyl]carbamoyl}azetidin-1-yl)-5-cyano-2-(trifluoromethyl)nicotinate

Prepared according to Method D from 1-[3-cyano-5-(ethoxycarbonyl)-6-(trifluoromethyl)pyridin-2-yl]azetidine-3-carboxylic acid and 5-chlorothiophene-2-sulfonamide to give ethyl 6-(3-{[(5-chloro-2-thienyl)sulfonyl]carbamoyl}azetidin-1-yl)-5-cyano-2-(trifluoromethyl)nicotinate. Yield: 43.9 mg (56%).

¹H NMR (500 MHz, DMSO-d₆) δ 1.38 (3H, t, J=7.1 Hz), 3.63 (1H, quintet, J=7.4 Hz), 4.36 (2H, q, J=7.2 Hz), 4.50-4.64 (4H, br s), 6.97 (1H, d, J=4.0 Hz), 7.70 (1H, d, J=4.2 Hz), 8.24 (1H, s), 11.48 (1H, s). MS m/z: 523 (M+1)

### Example 55

### Ethyl 5-cyano-6-[4-({[(4-fluorobenzyl)sulfonyl]amino}carbonyl)piperidin-1-yl]-2-(trifluoromethyl)nicotinate

### (a) 1-[3-Cyano-5-(ethoxycarbonyl)-6-(trifluoromethyl)pyridin-2-yl]piperidine-4-carboxylic acid

TEA (0.908 g, 8.97 mmol) was added to a suspension of ethyl 6-chloro-5-cyano-2-(trifluoromethyl)nicotinate (1.0 g, 3.59 mmol) and piperidine-4-carboxylic acid (0.510 g, 3.95 mmol) in EtOH (10 mL) and the mixture was heated in a single-node microwave oven for 15 minutes. The solvent was evaporated and the residue was partioned between iPrOAc (10 mL)/water and 20% Na₂CO₃ (1 mL). The aqueous phase was separated, 1 mL EtOH was added and the water phase was made acidic by addition of concentrated HCl. The acidic water phase was extracted with iPrOAc (2×10 mL). The organic phase was dried (MgSO₄), filtered and concentrated to give 1-[3-cyano-5-(ethoxycarbonyl)-6-(trifluoromethyl)pyridin-2-yl]piperidine-4-carboxylic acid as a brown solid which was used without further purification. Yield: 1.06 g (79%).

¹H NMR (500 MHz, DMSO-d₆) δ 1.28 (3H, t, J=7.1 Hz), 1.61-1.71 (2H, m), 1.95-2.02 (2H, m), 2.60-2.68 (1H, m), 3.31-3.38 (2H, m), 4.28 (2H, q, J=7.1 Hz), 4.41-4.48 (2H, m), 8.51 (1H, s).

### (b) Ethyl 5-cyano-6-[4-({[(4-fluorobenzyl)sulfonyl]amino}carbonyl)piperidin-1-yl]-2-(trifluoromethyl)nicotinate

Prepared according to Method C from 1-[3-cyano-5-(ethoxycarbonyl)-6-(trifluoromethyl)pyridin-2-yl]piperidine-4-carboxylic acid and 1-(4-fluorophenyl)methanesulfonamide to give ethyl 5-cyano-6-[4-({[(4-fluorobenzyl)sulfonyl]amino}carbonyl)piperidin-1-yl]-2-(trifluoromethyl)nicotinate. Yield: 4.3 mg (4%)

¹H NMR (600 MHz, CDCl₃) δ 1.36 (3H, t, J=7 Hz), 1.78-1.94 (4H, m), 2.49-2.55 (1H, m), 3.23 (2H, t, J=12.5 Hz), 4.35 (2H, q, J=7 Hz), 4.60 (2H, s), 4.67 (2H, br d, J=12.5 Hz), 7.06 (2H, t, J=8.5 Hz), 7.31 (2H, dd, J=5, 8.5 Hz), 8.34 (1H, s), 9.50 (1H, s). MS m/z: 543 (M+1)

### Example 56

### Ethyl 5-cyano-6-[4-({[(3-fluorobenzyl)sulfonyl]amino}carbonyl)piperidin-1-yl]-2-(trifluoromethyl)nicotinate

Prepared according to Method C from 1-[3-cyano-5-(ethoxycarbonyl)-6-(trifluoromethyl)pyridin-2-yl]piperidine-4-carboxylic acid and 1-(3-fluorophenyl)methanesulfonamide to give ethyl 5-cyano-6-[4-({[(3-fluorobenzyl)sulfonyl]amino}carbonyl)piperidin-1-yl]-2-(trifluoromethyl)nicotinate. Yield: 5.7 mg (5%).

¹H NMR (500 MHz, CDCl₃) δ 1.40 (3H, t, J=7.5 Hz), 1.81-1.97 (4H, m), 2.53-2.61 (1H, m), 3.28 (2H, t, J=12.5 Hz), 4.39 (2H, q, J=7.5 Hz), 4.67 (2H, s), 4.71 (2H, br d, J=12.5 Hz), 7.12-7.15 (3H, m), 7.36-7.41 (1H, m), 8.38 (1H, s), 9.68 (1H, s). MS m/z: 543 (M+1)

### Example 57

### Ethyl 5-cyano-6-[4-({[(2-fluorobenzyl)sulfonyl]amino}carbonyl)piperidin-1-yl]-2-(trifluoromethyl)nicotinate

Prepared according to Method C from 1-[3-cyano-5-(ethoxycarbonyl)-6-(trifluoromethyl)pyridin-2-yl]piperidine-4-carboxylic acid and 1-(2-fluorophenyl)methanesulfonamide to give ethyl 5-cyano-6-[4-({[(2-fluorobenzyl)sulfonyl]amino}carbonyl)piperidin-1-yl]-2-(trifluoromethyl)nicotinate. Yield: 5.1 mg (5%).

¹H NMR (400 MHz, CDCl₃) δ 1.35 (3H, t, J=6.5 Hz), 1.80-1.99 (4H, m), 2.53-2.61 (1H, m), 3.27 (2H, t, J=13 Hz), 4.34 (2H, q, J=6.5 Hz), 4.67 (2H, br d, J=13 Hz), 4.69 (2H, s), 7.11 (1H, t, J=9 Hz), 7.17 (1H, t, J=7.5 Hz), 7.34-7.39 (2H, m), 8.33 (1H, s), 9.63 (1H, s). MS m/z: 543 (M+1)

### Example 58

### Ethyl 5-cyano-6-[4-({[(4-methylbenzyl)sulfonyl]amino}carbonyl)piperidin-1-yl]-2-(trifluoromethyl)nicotinate

Prepared according to Method C from 1-[3-cyano-5-(ethoxycarbonyl)-6-(trifluoromethyl)pyridin-2-yl]piperidine-4-carboxylic acid and 1-(4-methylphenyl)methanesulfonamide to give ethyl 5-cyano-6-[4-({[(4-methylbenzyl)sulfonyl]amino}carbonyl)piperidin-1-yl]-2-(trifluoromethyl)nicotinate. Yield: 3.4 mg (3%).

¹H NMR (400 MHz, CDCl₃) δ 1.36 (3H, t, J=7.5 Hz), 1.75-1.93 (4H, m), 2.34 (3H, s), 2.44-2.52 (1H, m), 3.23 (2H, t, J=12.5 Hz), 4.35 (2H, q, J=7.5 Hz), 4.58 (2H, s), 4.66 (2H, br d, J=12.5 Hz), 7.15-7.21 (4H, m), 8.33 (1H, s), 8.88 (1H, s). MS m/z: 539 (M+1)

### Example 59

### Ethyl 5-cyano-6-[4-({[(3-methylbenzyl)sulfonyl]amino}carbonyl)piperidin-1-yl]-2-(trifluoromethyl)nicotinate

Prepared according to Method C from 1-[3-cyano-5-(ethoxycarbonyl)-6-(trifluoromethyl)pyridin-2-yl]piperidine-4-carboxylic acid and 1-(3-ethylphenyl)methanesulfonamide to give ethyl 5-cyano-6-[4-({[(3-ethylbenzyl)sulfonyl]amino}carbonyl)piperidin-1-yl]-2-(trifluoromethyl)nicotinate. Yield: 2.8 mg (3%).

¹H NMR (400 MHz, CDCl₃) [symbol] 1.31 (3H, t, J=7.5 Hz), 1.71-1.88 (4H, m), 2.28 (3H, s), 2.39-2.47 (1H, m), 3.18 (2H, t, J=13 Hz), 4.30 (2H, q, J=7.5 Hz), 4.54 (2H, s), 4.61 (2H, br d, J=13 Hz), 7.05-7.23 (4H, m), 8.29 (1H, s), 8.72 (1H, s). MS m/z: 539 (M+1)

### Example 60

### Ethyl 6-[4-({[(4-chlorobenzyl)sulfonyl]amino}carbonyl)piperidin-1-yl]-5-cyano-2-(trifluoromethyl)nicotinate

Prepared according to Method C from 1-[3-cyano-5-(ethoxycarbonyl)-6-(trifluoromethyl)pyridin-2-yl]piperidine-4-carboxylic acid and 1-(4-chlorophenyl)methanesulfonamide to give ethyl 6-[4-({[(4-chlorobenzyl)sulfonyl]amino}carbonyl)piperidin-1-yl]-5-cyano-2-(trifluoromethyl)nicotinate. Yield: 6.6 mg (6%).

¹H NMR (600 MHz, CDCl₃) δ 1.20 (3H, t, J=7.5 Hz), 1.63-1.70 (2H, m), 1.74-1.79 (2H, m), 2.39-2.41 (1H, m), 3.09 (2H, t, J=12.5 Hz), 4.18 (2H, q, J=7.5 Hz), 4.42 (2H, s), 4.52 (2H, br d, J=12.5 Hz), 7.12 (2H, d, J=8.5 Hz), 7.19 (2H, d, J=8.5 Hz), 8.18 (1H, s), 11.32 (1H, s). MS m/z: 559 (M+1)

### Example 61

### Ethyl 6-[4-({[(2-chlorobenzyl)sulfonyl]amino}carbonyl)piperidin-1-yl]-5-cyano-2-(trifluoromethyl)nicotinate

Prepared according to Method C from 1-[3-cyano-5-(ethoxycarbonyl)-6-(trifluoromethyl)pyridin-2-yl]piperidine-4-carboxylic acid and 1-(2-chlorophenyl)methanesulfonamide to give ethyl 6-[4-({[(2-chlorobenzyl)sulfonyl]amino}carbonyl)piperidin-1-yl]-5-cyano-2-(trifluoromethyl)nicotinate. Yield: 7.8 mg (7%).

¹H NMR (600 MHz, CDCl₃) δ 1.35 (3H, t, J=7 Hz), 1.81-1.90 (2H, m), 1.96-2.00 (2H, m), 2.56-2.64 (1H, m), 3.26 (2H, t, J=12 Hz), 4.34 (2H, q, J=7 Hz), 4.68 (2H, br d, J=12 Hz), 4.84 (2H, s), 7.27-7.34 (2H, m), 7.42 (2H, t, J=7 Hz), 8.34 (1H, s), 10.03 (1H, s). MS m/z: 559 (M+1)

### Example 62

### Ethyl 6-[4-({[(3-chlorobenzyl)sulfonyl]amino}carbonyl)piperidin-1-yl]-5-cyano-2-(trifluoromethyl)nicotinate

Prepared according to Method C from 1-[3-cyano-5-(ethoxycarbonyl)-6-(trifluoromethyl)pyridin-2-yl]piperidine-4-carboxylic acid and 1-(3-chlorophenyl)methanesulfonamide to give ethyl 6-[4-({[(3-chlorobenzyl)sulfonyl]amino}carbonyl)piperidin-1-yl]-5-cyano-2-(trifluoromethyl)nicotinate. Yield: 7.3 mg (6%).

¹H NMR (500 MHz, CDCl₃) δ 1.40 (3H, t, J=7.5 Hz), 1.81-1.90 (2H, m), 1.91-1.97 (2H, m), 2.54-2.62 (1H, m), 3.28 (2H, t, J=12.5 Hz), 4.39 (2H, q, J=7.5 Hz), 4.64 (2H, s), 4.72 (2H, br d, J=12.5 Hz), 7.25 (1H, d, J=7.5 Hz), 7.34-7.42 (3H, m), 8.38 (1H, s), 10.02 (1H, s). MS m/z: 559 (M+1)

### Example 63

### Ethyl 5-cyano-6-[4-({[(2,4-dichlorobenzyl)sulfonyl]amino}carbonyl)piperidin-1-yl]-2-(trifluoromethyl)nicotinate

Prepared according to Method C from 1-[3-cyano-5-(ethoxycarbonyl)-6-(trifluoromethyl)pyridin-2-yl]piperidine-4-carboxylic acid and 1-(2,4-dichlorophenyl)methanesulfonamide to give ethyl 5-cyano-6-[4-({[(2,4-dichlorobenzyl)sulfonyl]amino}carbonyl)piperidin-1-yl]-2-(trifluoromethyl)nicotinate. Yield: 5.5 mg (5%).

¹H NMR (600 MHz, CDCl₃) δ 1.35 (3H, t, J=7.5 Hz), 1.83-1.90 (2H, m), 1.97-2.01 (2H, m), 2.56-2.64 (1H, m), 3.29 (2H, t, J=12.5 Hz), 4.34 (2H, q, J=7.5 Hz), 4.68 (2H, br d, J=12.5 Hz), 4.80 (2H, s), 7.28 (1H, dd, J=2, 8.5 Hz), 7.37 (1H, d, J=8.5 Hz), 7.45 (1H, d, J=2 Hz), 8.33 (1H, s), 10.04 (1H, s). MS m/z: 593 (M+1).

### Example 64

### Ethyl 6-[4-({[(5-chloro-2-thienyl)sulfonyl]amino}carbonyl)piperidin-1-yl]-5-cyano-2-(trifluoromethyl)nicotinate

Prepared according to Method C from 1-[3-cyano-5-(ethoxycarbonyl)-6-(trifluoromethyl)pyridin-2-yl]piperidine-4-carboxylic acid and 5-chlorothiophene-2-sulfonamide to give ethyl 6-[4-({[(5-chloro-2-thienyl)sulfonyl]amino}carbonyl)piperidin-1-yl]-5-cyano-2-(trifluoromethyl)nicotinate. Yield: 19.1 mg (17%).

¹H NMR (400 MHz, CDCl₃) δ 1.34 (3H, t, J=7 Hz), 1.72-1.84 (2H, m), 1.91-1.97 (2H, m), 2.55-2.65 (1H, m), 3.27 (2H, t, J=12.5 Hz), 4.33 (2H, q, J=7.5 Hz), 4.61 (2H, br d, J=12.5 Hz), 6.91 (1H, d, J=4 Hz), 7.62 (1H, d, J=4 Hz), 8.30 (1H, s), 10.99 (1H, s). MS m/z: 551 (M+1)

### Example 65

### Ethyl 5-cyano-6-[3-({[(2-fluorobenzyl)sulfonyl]amino}carbonyl)azetidin-1-yl]-2-(fluoromethyl)nicotinate

### (a) 1-[3-Cyano-5-(ethoxycarbonyl)-6-(fluoromethyl)pyridin-2-yl]azetidine-3-carboxylic acid

TEA (653 mg, 6.46 mmol) was added to a solution of ethyl 6-chloro-5-cyano-2-(fluoromethyl)nicotinate (400 mg, 1.61 mmol) and azetidine-3-carboxylic acid (179 mg, 1.78 mmol) in water/EtOH (4.5 mL). The mixture was heated in a single-node microwave oven at 120° C. for 20 minutes. The solvent was evaporated and the residue was taken up in DCM and washed with 1% KHSO₄. The aqueous phase was extracted with DCM and the combined organic phase was filtered through a phase separator and concentrated. Purification by HPLC (Kromasil C₈, 10 μm, Eluent: A gradient of 5% CH₃CN to 100% CH₃CN/(0.2% cOH(aq)) gave 1-[3-cyano-5-(ethoxycarbonyl)-6-(fluoromethyl)pyridin-2-yl]azetidine-3-carboxylic acid as a white solid. Yield: 302 mg (60%).

¹H NMR (400 MHz, CDCl₃) [symbol] 1.31 (3H, t, J=7.3 Hz), 3.59-3.69 (1H, m), 4.31 (2H, q, J=7.3 Hz), 4.60-4.70 (4H, m), 5.69 (2H, d, J=47.3 Hz), 8.30 (1H, br s).

### (b) Ethyl 5-cyano-6-[3-({[(2-fluorobenzyl)sulfonyl]amino}carbonyl)azetidin-1-yl]-2-(fluoromethyl)nicotinate

Prepared according to Method E from 1-[3-cyano-5-(ethoxycarbonyl)-6-(fluoromethyl)pyridin-2-yl]azetidine-3-carboxylic acid and 1-(2-fluorophenyl)methanesulfonamide to give ethyl 5-cyano-6-[3-({[(2-fluorobenzyl)sulfonyl]amino}carbonyl)azetidin-1-yl]-2-(fluoromethyl)nicotinate. Yield: 21 g (44%).

¹H NMR (400 MHz, DMSO-d₆) δ 1.30 (3H, t, J=7.2 Hz), 3.55-3.66 (1H, m), 4.25 (2H, q, J=7.2 Hz), 4.34-4.44 (2H, m), 4.43-4.56 (2H, m), 4.80 (2H, s), 5.68 (2H, d, J=47.1 Hz), 7.18-7.32 (2H, m), 7.37-7.52 (2H, m), 8.39 (1H, s), 11.80-12.19 (1H, m). MS m/z: 479 (M+1).

### Example 66

### Ethyl 5-cyano-6-[3-({[(3-fluorobenzyl)sulfonyl]amino}carbonyl)azetidin-1-yl]-2-(fluoromethyl)nicotinate

Prepared according to Method E from 1-[3-cyano-5-(ethoxycarbonyl)-6-(fluoromethyl)pyridin-2-yl]azetidine-3-carboxylic acid and 1-(3-fluorophenyl)methanesulfonamide to give ethyl 5-cyano-6-[3-({[(3-fluorobenzyl)sulfonyl]amino}carbonyl)azetidin-1-yl]-2-(fluoromethyl)nicotinate. Yield: 25 g (53%).

¹H NMR (400 MHz, DMSO-d₆) δ 1.29 (3H, t, J=7.1 Hz), 3.54-3.64 (1H, m), 4.24 (2H, q, J=7.1 Hz), 4.28-4.36 (2H, m), 4.39-4.53 (2H, m), 4.79 (2H, s), 5.67 (2H, d, J=7.1 Hz), 7.13-7.27 (3H, m), 7.37-7.47 (1H, m), 8.38 (1H, s), 11.55-12.36 (1H, m) MS m/z: 479 (M+1).

### Example 67

### Ethyl 5-cyano-6-[3-({[(4-fluorobenzyl)sulfonyl]amino}carbonyl)azetidin-1-yl]-2-(fluoromethyl)nicotinate

Prepared according to Method E from 1-[3-cyano-5-(ethoxycarbonyl)-6-(fluoromethyl)pyridin-2-yl]azetidine-3-carboxylic acid and 1-(4-fluorophenyl)methanesulfonamide to give ethyl 5-cyano-6-[3-({[(4-fluorobenzyl)sulfonyl]amino}carbonyl)azetidin-1-yl]-2-(fluoromethyl)nicotinate. Yield: 27 g (56%).

¹H NMR (400 MHz, DMSO-d₆) δ 1.29 (3H, t, J=7.2 Hz), 3.55-3.77 (1H, m), 4.24 (2H, q, J=7.1 Hz), 4.29-4.37 (2H, m), 4.41-4.51 (2H, m), 4.73 (2H, s), 5.66 (2H, d, J=7.1 Hz), 7.15-7.23 (2H, m), 7.34-7.42 (2H, m), 8.37 (1H, s). MS m/z: 479 (M+1).

### Example 68

### Ethyl 6-[3-({[(2-chlorobenzyl)sulfonyl]amino}carbonyl)azetidin-1-yl]-5-cyano-2-(fluoromethyl)nicotinate

Prepared according to Method E from 1-[3-cyano-5-(ethoxycarbonyl)-6-(fluoromethyl)pyridin-2-yl]azetidine-3-carboxylic acid and 1-(2-chlorophenyl)methanesulfonamide to give ethyl 6-[3-({[(2-chlorobenzyl)sulfonyl]amino}carbonyl)azetidin-1-yl]-5-cyano-2-(fluoromethyl)nicotinate. Yield: 13 mg (27%).

¹H NMR (400 MHz, DMSO-d₆) [symbol] 1.30 (3H, t, J=7.2 Hz), 3.59-3.69 (1H, m), 4.25 (2H, q, J=7.2 Hz), 4.36-4.56 (4H, m), 4.90 (2H, s), 5.67 (2H, d, J=47.3 Hz), 7.34-7.56 (4H, m), 8.38 (1H, s), 11.73-12.28 (1H, m). MS m/z: 495 (M+1).

### Example 69

### Ethyl 6-[3-({[(3-chlorobenzyl)sulfonyl]amino}carbonyl)azetidin-1-yl]-5-cyano-2-(fluoromethyl)nicotinate

Prepared according to Method E from 1-[3-cyano-5-(ethoxycarbonyl)-6-(fluoromethyl)pyridin-2-yl]azetidine-3-carboxylic acid and 1-(3-chlorophenyl)methanesulfonamide to give ethyl 6-[3-({[(3-chlorobenzyl)sulfonyl]amino}carbonyl)azetidin-1-yl]-5-cyano-2-(fluoromethyl)nicotinate. Yield: 28 mg (58%).

¹H NMR (400 MHz, DMSO-d₆) [symbol] 1.30 (3H, t, J=7.2 Hz), 3.51-3.65 (1H, m), 4.25 (2H, q, J=7.2 Hz), 4.27-4.37 (2H, m), 4.40-4.53 (2H, m), 4.79 (2H, s), 5.67 (2H, d, J=47.1 Hz), 7.27-7.50 (4H, m), 8.36-8.40 (1H, m), 11.71-12.13 (1H, m). MS m/z: 495 (M+1).

### Example 70

### Ethyl 6-[3-({[(4-chlorobenzyl)sulfonyl]amino}carbonyl)azetidin-1-yl]-5-cyano-2-(fluoromethyl)nicotinate

Prepared according to Method E from 1-[3-cyano-5-(ethoxycarbonyl)-6-(fluoromethyl)pyridin-2-yl]azetidine-3-carboxylic acid and 1-(4-chlorophenyl)methanesulfonamide to give ethyl 6-[3-({[(4-chlorobenzyl)sulfonyl]amino}carbonyl)azetidin-1-yl]-5-cyano-2-(fluoromethyl)nicotinate. Yield: 33 mg (68%).

¹H NMR (400 MHz, DMSO-d₆) δ 1.29 (3H, t, J=7.2 Hz), 3.45-3.58 (1H, m), 4.24 (2H, q, J=7.2 Hz), 4.29-4.38 (2H, m), 4.38-4.50 (2H, m), 4.60 (2H, s), 5.66 (2H, d, J=47.1 Hz), 7.29-7.41 (4H, m), 8.36 (1H, s). MS m/z: 495 (M+1).

### Example 71

### Ethyl 5-cyano-2-(fluoromethyl)-6-[3-({[(3-methylbenzyl)sulfonyl]amino}carbonyl)azetidin-1-yl]nicotinate

Prepared according to Method E from 1-[3-cyano-5-(ethoxycarbonyl)-6-(fluoromethyl)pyridin-2-yl]azetidine-3-carboxylic acid and 1-(3-methylphenyl)methanesulfonamide to give ethyl 5-cyano-2-(fluoromethyl)-6-[3-({[(3-methylbenzyl)sulfonyl]amino}carbonyl)azetidin-1-yl]nicotinate. Yield: 41 mg (86%).

¹H NMR (400 MHz, DMSO-d₆) δ 1.30 (3H, t, J=7.2 Hz), 2.27 (3H, s), 3.51-3.60 (1H, m), 4.25 (2H, q, J=7.2 Hz), 4.29-4.37 (2H, m), 4.39-4.51 (2H, m), 4.69 (2H, s), 5.67 (2H, d, J=50.0 Hz), 7.07-7.32 (4H, m), 8.38 (1H, s), 11.59-12.03 (1H, m). MS m/z: 475 (M+1).

### Example 72

### Ethyl 5-cyano-2-(fluoromethyl)-6-[3-({[(4-methylbenzyl)sulfonyl]amino}carbonyl)azetidin-1-yl]nicotinate

Prepared according to Method E from 1-[3-cyano-5-(ethoxycarbonyl)-6-(fluoromethyl)pyridin-2-yl]azetidine-3-carboxylic acid and 1-(4-methylphenyl)methanesulfonamide to give ethyl 5-cyano-2-(fluoromethyl)-6-[3-({[(4-methylbenzyl)sulfonyl]amino}carbonyl)azetidin-1-yl]nicotinate. Yield: 12 mg (25%).

¹H NMR (400 MHz, DMSO-d₆) δ 1.29 (3H, t, J=7.2 Hz), 2.28 (3H, s), 3.53-3.60 (1H, m), 4.24 (2H, q, J=7.2 Hz), 4.29-4.36 (2H, m), 4.39-4.50 (2H, m), 4.67 (2H, s), 5.67 (2H, d, J=47.1 Hz), 7.15-7.23 (4H, m), 8.37-8.40 (1H, m), 11.48-12.04 (1H, m) MS m/z: 475 (M+1).

### Example 73

### Ethyl 5-cyano-6-[3-({[(2,4-dichlorobenzyl)sulfonyl]amino}carbonyl)azetidin-1-yl]-2-(fluoromethyl)nicotinate

Prepared according to Method E from 1-[3-cyano-5-(ethoxycarbonyl)-6-(fluoromethyl)pyridin-2-yl]azetidine-3-carboxylic acid and 1-(2,4-dichlorophenyl)methanesulfonamide to give ethyl 5-cyano-6-[3-({[(2,4-dichlorobenzyl)sulfonyl]amino}carbonyl)azetidin-1-yl]-2-(fluoromethyl)nicotinate. Yield: 27 mg (51%).

¹H NMR (400 MHz, DMSO-d₆) δ 1.29 (3H, t, J=7.2 Hz), 3.56-3.65 (1H, m), 4.24 (2H, q, J=7.2 Hz), 4.35-4.58 (4H, m), 4.86 (2H, s), 5.67 (2H, d, J=47.1 Hz), 7.41-7.70 (3H, m), 8.36-8.39 (1H, m). MS m/z: 529 (M+1).

### Example 74

### Ethyl 5-cyano-2-(fluoromethyl)-6-{3-[({[(4-methylcyclohexyl)methyl]sulfonyl}amino)carbonyl]azetidin-1-yl}nicotinate

Prepared according to Method E from 1-[3-cyano-5-(ethoxycarbonyl)-6-(fluoromethyl)pyridin-2-yl]azetidine-3-carboxylic acid and 1-(4-methylcyclohexyl)methanesulfonamide to give ethyl 5-cyano-6-[3-({[(2,4-dichlorobenzyl)sulfonyl]amino}carbonyl)azetidin-1-yl]-2-(fluoromethyl)nicotinate. Yield: 28 mg (57%).

¹H NMR (400 MHz, DMSO-d₆) δ 0.75-0.92 (4H, m), 0.95-1.17 (3H, m), 1.25 (3H, t, J=7.1 Hz), 1.35-1.54 (4H, m), 1.55-1.64 (1H, m), 1.74-1.84 (1H, m), 2.00-2.10 (1H, m), 3.22-3.28 (1H, m), 3.51-3.63 (1H, m), 4.20 (2H, q, J=7.1 Hz), 4.29-4.39 (2H, m), 4.40-4.51 (2H, m), 5.61 (2H, d, J=47.3 Hz), 8.32 (1H, s). MS m/z: 481 (M+1).

### Example 75

### Ethyl 5-cyano-6-[4-({[(2-fluorobenzyl)sulfonyl]amino}carbonyl)piperidin-1-yl]-2-(fluoromethyl)nicotinate

### (a) 1-[3-Cyano-5-(ethoxycarbonyl)-6-(fluoromethyl)pyridin-2-yl]piperidine-4-carboxylic acid

TEA (653 mg, 6.46 mmol) was added to a solution of ethyl 6-chloro-5-cyano-2-(fluoromethyl)nicotinate (400 mg, 1.61 mmol) and piperidine-4-carboxylic acid (229 mg, 1.78 mmol) in water/EtOH (4.5 mL). The mixture was heated in a single-node microwave oven at 120° C. for 20 minutes. The solvent was evaporated and the residue was taken up in DCM and washed with 1% KHSO₄. The aqueous phase was extracted with DCM and the combined organic phase was filtered through a phase separator and concentrated. Purification by HPLC (Kromasil C₈, Eluent: A gradient of 5% CH₃CN to 100% CH₃CN/(0.2% HOAc(aq)) gave 1-[3-cyano-5-(ethoxycarbonyl)-6-(fluoromethyl)pyridin-2-yl]azetidine-3-carboxylic acid as a white solid. Yield: 76 mg (14%).

¹H NMR (400 MHz, CDCl₃) δ 1.36 (3H, t, J=7.2 Hz), 1.82-1.94 (2H, m), 2.05-2.14 (2H, m), 2.66-2.76 (1H, m), 3.32-3.42 (2H, m), 4.31 (2H, t, J=7.2 Hz), 4.61-4.69 (2H, m), 5.70 (2H, d, J=47.3 Hz), 8.36 (1H, br s).

### (b) Ethyl 5-cyano-6-[4-({[(2-fluorobenzyl)sulfonyl]amino}carbonyl)piperidin-1-yl]-2-(fluoromethyl)nicotinate

Prepared according to Method E from 1-[3-cyano-5-(ethoxycarbonyl)-6-(fluoromethyl)pyridin-2-yl]piperidine-4-carboxylic acid and 1-(2-fluorophenyl)methanesulfonamide to give ethyl 5-cyano-6-[4-({[(2-fluorobenzyl)sulfonyl]amino}carbonyl)piperidin-1-yl]-2-(fluoromethyl)nicotinate. Yield: 13 mg (25%).

¹H NMR (400 MHz, DMSO-d₆) δ 1.29 (3H, t, J=7.1 Hz), 1.56-1.75 (2H, m), 1.82-1.93 (2H, m), 2.56-2.64 (1H, m), 3.14-3.26 (2H, m), 4.25 (2H, q, J=7.1 Hz), 4.55-4.64 (2H, m), 4.68 (2H, s), 5.68 (2H, d, J=47.1 Hz), 7.18-7.30 (2H, m), 7.32-7.48 (2H, m), 8.39 (1H, s). MS m/z: 507 (M+1).

### Example 76

### Ethyl 5-cyano-6-[4-({[(3-fluorobenzyl)sulfonyl]amino}carbonyl)piperidin-1-yl]-2-(fluoromethyl)nicotinate

Prepared according to Method E from 1-[3-cyano-5-(ethoxycarbonyl)-6-(fluoromethyl)pyridin-2-yl]piperidine-4-carboxylic acid and 1-(3-fluorophenyl)methanesulfonamide to give ethyl 5-cyano-6-[4-({[(3-fluorobenzyl)sulfonyl]amino}carbonyl)piperidin-1-yl]-2-(fluoromethyl)nicotinate. Yield: 16 mg (31%).

¹H NMR (400 MHz, DMSO-d₆) [symbol] 1.30 (3H, t, J=7.1 Hz), 1.56-1.71 (2H, m), 1.79-1.89 (2H, m), 2.55-2.61 (1H, m), 3.15-3.26 (2H, m), 4.25 (2H, q, J=7.1 Hz), 4.53-4.64 (2H, m), 4.70 (2H, s), 5.69 (2H, d, J=47.1 Hz), 7.07-7.17 (2H, m), 7.20-7.28 (1H, m), 7.39-7.49 (1H, m), 8.39-8.42 (1H, m), 11.47-12.06 (1H, m). MS m/z: 507 (M+1).

### Example 77

### Ethyl 5-cyano-6-[4-({[(4-fluorobenzyl)sulfonyl]amino}carbonyl)piperidin-1-yl]-2-(fluoromethyl)nicotinate

Prepared according to Method E from 1-[3-cyano-5-(ethoxycarbonyl)-6-(fluoromethyl)pyridin-2-yl]piperidine-4-carboxylic acid and 1-(4-fluorophenyl)methanesulfonamide to give ethyl 5-cyano-6-[4-({[(4-fluorobenzyl)sulfonyl]amino}carbonyl)piperidin-1-yl]-2-(fluoromethyl)nicotinate. Yield: 23 mg (45%).

¹H NMR (400 MHz, DMSO-d₆) δ 1.29 (3H, t, J=7.1 Hz), 1.56-1.70 (2H, m), 1.78-1.89 (2H, m), 2.52-2.56 (1H, m), 3.14-3.24 (2H, m), 4.25 (2H, q, J=7.1 Hz), 4.51-4.63 (4H, m), 5.68 (2H, d, J=47.1 Hz), 7.16-7.24 (2H, m), 7.27-7.34 (2H, m), 8.39 (1H, s). MS m/z: 507 (M+1).

### Example 78

### Ethyl 6-[4-({[(2-chlorobenzyl)sulfonyl]amino}carbonyl)piperidin-1-yl]-5-cyano-2-(fluoromethyl)nicotinate

Prepared according to Method E from 1-[3-cyano-5-(ethoxycarbonyl)-6-(fluoromethyl)pyridin-2-yl]piperidine-4-carboxylic acid and 1-(2-chlorophenyl)methanesulfonamide to give ethyl 6-[4-({[(2-chlorobenzyl)sulfonyl]amino}carbonyl)piperidin-1-yl]-5-cyano-2-(fluoromethyl)nicotinate. Yield: 24 mg (45%).

¹H NMR (400 MHz, DMSO-d₆) δ 1.29 (3H, t, J=7.2 Hz), 1.56-1.74 (2H, m), 1.84-1.95 (2H, m), 2.56-2.66 (1H, m), 3.16-3.27 (2H, m), 4.25 (2H, q, J=7.2 Hz), 4.54-4.65 (2H, m), 4.80 (2H, s), 5.68 (2H, d, J=47.3 Hz), 7.35-7.46 (3H, m), 7.48-7.55 (1H, m), 8.39 (1H, s). MS m/z: 523 (M+1).

### Example 79

### Ethyl 6-[4-({[(3-chlorobenzyl)sulfonyl]amino}carbonyl)piperidin-1-yl]-5-cyano-2-(fluoromethyl)nicotinate

Prepared according to Method E from 1-[3-cyano-5-(ethoxycarbonyl)-6-(fluoromethyl)pyridin-2-yl]piperidine-4-carboxylic acid and 1-(3-chlorophenyl)methanesulfonamide to give ethyl 6-[4-({[(3-chlorobenzyl)sulfonyl]amino}carbonyl)piperidin-1-yl]-5-cyano-2-(fluoromethyl)nicotinate. Yield: 24 mg (46%).

¹H NMR (400 MHz, DMSO-d₆) [symbol] 1.30 (3H, t, J=7.1 Hz), 1.57-1.70 (2H, m), 1.76-1.88 (2H, m), 2.53-2.61 (1H, m), 3.15-3.27 (2H, m), 4.25 (2H, q, J=7.1 Hz), 4.55-4.63 (2H, m), 4.68 (2H, s), 5.68 (2H, d, J=47.3 Hz), 7.18-7.52 (4H, m), 8.40 (1H, s). MS m/z: 523 (M+1).

### Example 80

### Ethyl 6-[4-({[(4-chlorobenzyl)sulfonyl]amino}carbonyl)piperidin-1-yl]-5-cyano-2-(fluoromethyl)nicotinate

Prepared according to Method E from 1-[3-cyano-5-(ethoxycarbonyl)-6-(fluoromethyl)pyridin-2-yl]piperidine-4-carboxylic acid and 1-(4-chlorophenyl)methanesulfonamide to give ethyl 6-[4-({[(4-chlorobenzyl)sulfonyl]amino}carbonyl)piperidin-1-yl]-5-cyano-2-(fluoromethyl)nicotinate. Yield: 24 mg (46%).

¹H NMR (400 MHz, DMSO-d₆) δ 1.30 (3H, t, J=7.2 Hz), 1.56-1.71 (2H, m), 1.80-1.90 (2H, m), 2.54-2.60 (1H, m), 3.13-3.26 (2H, m), 4.25 (2H, q, J=7.1 Hz), 4.55-4.63 (2H, m), 4.66 (2H, s), 5.68 (2H, d, J=47.1 Hz), 7.30 (2H, d, J=8.5 Hz), 7.46 (2H, d, J=8.5 Hz), 8.38-8.41 (1H, m). MS m/z: 523 (M+1).

### Example 81

### Ethyl 5-cyano-2-(fluoromethyl)-6-[4-({[(3-methylbenzyl)sulfonyl]amino}carbonyl)piperidin-1-yl]nicotinate

Prepared according to Method E from 1-[3-cyano-5-(ethoxycarbonyl)-6-(fluoromethyl)pyridin-2-yl]piperidine-4-carboxylic acid and 1-(3-methylphenyl)methanesulfonamide to give ethyl 5-cyano-2-(fluoromethyl)-6-[4-({[(3-methylbenzyl)sulfonyl]amino}carbonyl)piperidin-1-yl]nicotinate. Yield: 6 mg (12%).

¹H NMR (400 MHz, DMSO-d₆) [symbol] 1.30 (3H, t, J=7.1 Hz), 1.58-1.71 (2H, m), 1.79-1.88 (2H, m), 2.28 (3H, s), 2.52-2.58 (1H, m), 3.17-3.23 (2H, m), 4.25 (2H, q, J=7.1 Hz), 4.48-4.68 (4H, m), 5.68 (2H, d, J=47.1 Hz), 7.00-7.32 (4H, m), 8.40 (1H, s), 11.27-11.80 (1H, m). MS m/z: 503 (M+1).

### Example 82

### Ethyl 5-cyano-2-(fluoromethyl)-6-[4-({[(4-methylbenzyl)sulfonyl]amino}carbonyl)piperidin-1-yl]nicotinate

Prepared according to Method E from 1-[3-cyano-5-(ethoxycarbonyl)-6-(fluoromethyl)pyridin-2-yl]piperidine-4-carboxylic acid and 1-(4-methylphenyl)methanesulfonamide to give ethyl 5-cyano-2-(fluoromethyl)-6-[4-({[(4-methylbenzyl)sulfonyl]amino}carbonyl)piperidin-1-yl]nicotinate. Yield: 20 mg (40%).

¹H NMR (400 MHz, DMSO-d₆) δ 1.30 (3H, t, J=7.2 Hz), 1.57-1.72 (2H, m), 1.80-1.92 (2H, m), 2.30 (3H, s), 2.54-2.64 (1H, m), 3.11-3.25 (2H, m), 4.26 (2H, q, J=7.2 Hz), 4.52-4.68 (4H, m), 5.69 (2H, d, J=47.3 Hz), 7.11-7.28 (4H, m), 8.41 (1H, s), 11.33-11.86 (1H, m). MS m/z: 503 (M+1).

### Example 83

### Ethyl 5-cyano-6-[4-({[(2,4-dichlorobenzyl)sulfonyl]amino}carbonyl)piperidin-1-yl]-2-(fluoromethyl)nicotinate

Prepared according to Method E from 1-[3-cyano-5-(ethoxycarbonyl)-6-(fluoromethyl)pyridin-2-yl]piperidine-4-carboxylic acid and 1-(2,4-dichlorophenyl)methanesulfonamide to give ethyl 5-cyano-6-[4-({[(2,4-dichlorobenzyl)sulfonyl]amino}carbonyl)piperidin-1-yl]-2-(fluoromethyl)nicotinate. Yield: 21 mg (38%).

¹H NMR (400 MHz, DMSO-d₆) δ 1.30 (3H, t, J=7.2 Hz), 1.56-1.72 (2H, m), 1.83-1.94 (2H, m), 2.54-2.59 (1H, m), 3.15-3.27 (2H, m), 4.25 (2H, q, J=7.2 Hz), 4.53-4.63 (2H, m), 4.73 (2H, s), 5.68 (2H, d, J=47.3 Hz), 7.39-7.53 (2H, m), 7.62-7.70 (1H, m), 8.35-8.43 (1H, m). MS m/z: 557 (M+1).

### Example 84

### Ethyl 5-cyano-2-(fluoromethyl)-6-{4-[({[(4-methylcyclohexyl)methyl]sulfonyl}amino)carbonyl]piperidin-1-yl}nicotinate

Prepared according to Method E from 1-[3-cyano-5-(ethoxycarbonyl)-6-(fluoromethyl)pyridin-2-yl]piperidine-4-carboxylic acid and 1-(4-methylcyclohexyl)methanesulfonamide to give ethyl 5-cyano-2-(fluoromethyl)-6-{4-[({[(4-methylcyclohexyl)methyl]sulfonyl}amino)carbonyl]piperidin-1-yl}nicotinate. Yield: 18 mg (36%).

¹H NMR (400 MHz, DMSO-d₆) δ 0.80-0.90 (4H, m), 0.96-1.20 (3H, m), 1.29 (3H, t, J=7.2 Hz), 1.38-1.69 (7H, m), 1.77-1.97 (3H, m), 1.99-2.09 (1H, m), 2.59-2.71 (2H, m), 3.16-3.29 (2H, m), 4.25 (2H, q, J=7.2 Hz), 4.51-4.66 (2H, m), 5.67 (2H, d, J=47.3 Hz), 8.39 (1H, s). MS m/z: 509 (M+1).

### Example 85

### Ethyl 6-(3-{2-[(benzylsulfonyl)amino]-2-oxoethyl}azetidin-1-yl)-5-cyano-2-(difluoromethyl)nicotinate

### (a) tert-butyl 3-{2-[(benzylsulfonyl)amino]-2-oxoethyl}azetidine-1-carboxylate

DIPEA (0.3 mL, 1.72 mmol) was added to a mixture of [1-(tert-butoxycarbonyl)azetidin-3-yl]acetic acid (193 mg, 0.90 mmol) and TBTU (326 mg, 1.02 mmol) in dry DCM (4 mL). The reaction mixture was stirred at rt for 1 h and 1-phenylmethanesulfonamide (169 mg, 0.99 mmol) was added and the stirring was continued at r.t for 19 h. NaHCO₃(aq) was added and the mixture was extracted with EtOAc (3 times). The combined organic layer was dried over anhydrous MgSO₄, filtered and evaporated to give tert-butyl 3-{2-[(benzylsulfonyl)amino]-2-oxoethyl}azetidine-1-carboxylate which was used in the next step without further purification. Yield: 383 mg (116%). MS m/z: 367 (M−1).

### (b) 2-azetidin-3-yl-N-(benzylsulfonyl)acetamide

The crude tert-butyl 3-{2-[(benzylsulfonyl)amino]-2-oxoethyl}azetidine-1-carboxylate from the previous step (383 mg, 0.90 mmol) was dissolved in DCM (5 mL) and TFA (4 mL) was added. The reaction mixture was stirred at r.t for 1.5 hours. The solvent was evaporated to give 2-azetidin-3-yl-N-(benzylsulfonyl)acetamide which was used in the next step without further purification. Yield: 240 mg (100%). MS m/z: 269 (M+1), 267 (M−1).

### (c) Ethyl 6-(3-{2-[(benzylsulfonyl)amino]-2-oxoethyl}azetidin-1-yl)-5-cyano-2-(difluoromethyl)nicotinate

DIPEA (1 mL) was added to a solution of the crude 2-azetidin-3-yl-N-(benzylsulfonyl)acetamide from the previous step and ethyl 6-chloro-5-cyano-2-(difluoromethyl)nicotinate (180 mg, 0.69 mmol) in EtOH (9 mL). The reaction mixture was heated to 120° C. for 5 min using microwave single node heating. NaHCO₃(aq) was added and the mixture was extracted with DCM (3 times). The combined organic layer was run through a phase separator and evaporated. The crude product was purified by HPLC (Kromasil C₈ 10 μm, 21.5×250 mm using a gradient of CH₃CN/0.1 M NH₄OAc 20% to 50%, flow 25 mL/min) to give ethyl 6-(3-{2-[(benzylsulfonyl)amino]-2-oxoethyl}azetidin-1-yl)-5-cyano-2-(difluoromethyl)nicotinate. Yield: 156 mg (46% over 3 steps).

¹H NMR (500 MHz, DMSO-d₆): δ 1.31 (3H, t, J=7.1 Hz), 2.71 (2H, d, J=7.6 Hz), 3.04-3.11 (1H, m), 4.08 (2H, apparent br s), 4.28 (2H, q, J=7.1 Hz), 4.52 (2H, apparent br s), 4.70 (2H, s), 7.29-7.32 (2H, m), 7.37-7.44 (3H, m), 7.40 (1H, t, J=53 Hz, —CHF₂), 8.44 (1H, s), 11.68 (1H, s). MS m/z: 493 (M+1), 491 (M−1).

### Example 86

### Ethyl 5-cyano-6-(3-{[(2-cyanobenzyl)sulfonyl]carbamoyl}azetidin-1-yl)-2-(trifluoromethyl)nicotinate

Prepared according to Method D from 1-[3-cyano-5-(ethoxycarbonyl)-6-(trifluoromethyl)pyridin-2-yl]azetidine-3-carboxylic acid and 1-(2-cyanophenyl)methanesulfonamide to give ethyl 5-cyano-6-(3-{[(2-cyanobenzyl)sulfonyl]carbamoyl}azetidin-1-yl)-2-(trifluoromethyl)nicotinate. Yield: 45 mg (58%).

¹H NMR (500 MHz, CDCl₃) [symbol] 1.38 (3H, t, J=7.1 Hz), 3.70 (1H, tt, J=8.7, 6.1 Hz), 4.37 (2H, q, J=7.2 Hz), 4.55-4.70 (4H, m), 4.91 (2H, s), 7.55 (1H, t, J=7.5 Hz), 7.64 (1H, d, J=7.1 Hz), 7.69 (1H, t, J=7.6 Hz), 7.75 (1H, d, J=7.6 Hz), 8.26 (1H, s), 11.20 (1H, br s). MS m/z: 522 (M+1).

### Example 87

### Ethyl 5-cyano-6-(3-{[(2,6-difluorobenzyl)sulfonyl]carbamoyl}azetidin-1-yl)-2-(fluoromethyl)nicotinate

Prepared according to Method A from 1-[3-cyano-5-(ethoxycarbonyl)-6-(fluoromethyl)pyridin-2-yl]azetidine-3-carboxylic acid and 1-(2,6-difluorophenyl)methanesulfonamide to give ethyl 5-cyano-6-(3-{[(2,6-difluorobenzyl)sulfonyl]carbamoyl}azetidin-1-yl)-2-(fluoromethyl)nicotinate. Yield: 6.2 mg (12%).

¹H NMR (600 MHz, DMSO-d₆) δ 1.27 (3H, t, J=7.2 Hz), 3.55-3.62 (1H, m), 4.22 (2H, q, J=7.3 Hz), 4.31-4.42 (2H, m), 4.42-4.54 (2H, m), 4.77 (2H, s), 5.64 (2H, d, J=47.8 Hz), 7.11-7.19 (2H, m), 7.46-7.53 (1H, m), 8.36 (1H, s). MS m/z: 497 (M+1).

### Example 88

### Ethyl 5-cyano-2-(fluoromethyl)-6-(3-{[(4-fluoro-3-methylbenzyl)sulfonyl]carbamoyl}azetidin-1-yl)nicotinate

Prepared according to Method A from 1-[3-cyano-5-(ethoxycarbonyl)-6-(fluoromethyl)pyridin-2-yl]azetidine-3-carboxylic acid and 1-(4-fluoro-3-methylphenyl)methanesulfonamide to give ethyl 5-cyano-2-(fluoromethyl)-6-(3-{[(4-fluoro-3-methylbenzyl)sulfonyl]carbamoyl}azetidin-1-yl)nicotinate. Yield: 17.1 mg (35%).

¹H NMR (600 MHz, DMSO-d₆) δ 1.25 (3H, t, J=6.9 Hz), 2.15 (3H, s), 3.50-3.57 (1H, m), 4.20 (2H, q, J=7.4 Hz), 4.23-4.33 (2H, m), 4.32-4.47 (2H, m), 4.65 (2H, s), 5.63 (2H, d, J=46.8 Hz), 7.05-7.21 (3H, m), 8.34 (1H, s). MS m/z: 493 (M+1).

### Example 89

### Ethyl 6-(3-{[(2-chloro-4-fluorobenzyl)sulfonyl]carbamoyl}azetidin-1-yl)-5-cyano-2-(fluoromethyl)nicotinate

Prepared according to Method A from 1-[3-cyano-5-(ethoxycarbonyl)-6-(fluoromethyl)pyridin-2-yl]azetidine-3-carboxylic acid and 1-(2-chloro-4-fluorophenyl)methanesulfonamide to give ethyl 6-(3-{[(2-chloro-4-fluorobenzyl)sulfonyl]carbamoyl}azetidin-1-yl)-5-cyano-2-(fluoromethyl)nicotinate. Yield: 18.7 mg (36%).

¹H NMR (600 MHz, DMSO-d₆) δ 1.27 (3H, t, J=7.1 Hz), 3.56-3.63 (1H, m), 4.22 (2H, q, J=7.0 Hz), 4.32-4.51 (4H, m), 4.86 (2H, s), 5.64 (2H, d, J=46.5 Hz), 7.24-7.30 (1H, m), 7.47-7.57 (2H, m), 8.35 (1H, s). MS m/z: 513 (M+1).

### Example 90

### Ethyl 5-cyano-2-(fluoromethyl)-6-(3-{[(2,3,6-trifluorobenzyl)sulfonyl]carbamoyl}azetidin-1-yl)nicotinate

Prepared according to Method A from 1-[3-cyano-5-(ethoxycarbonyl)-6-(fluoromethyl)pyridin-2-yl]azetidine-3-carboxylic acid and 1-(2,3,6-trifluorophenyl)methanesulfonamide to give Ethyl 5-cyano-2-(fluoromethyl)-6-(3-{[(2,3,6-trifluorobenzyl)sulfonyl]carbamoyl}azetidin-1-yl)nicotinate. Yield: 24.4 mg (47%).

¹H NMR (600 MHz, DMSO-d₆) δ 1.25 (3H, t, J=7.2 Hz), 3.55-3.62 (1H, m), 4.20 (2H, q, J=7.1 Hz), 4.30-4.52 (4H, m), 4.82 (2H, s), 5.63 (2H, d, J=46.1 Hz), 7.16-7.23 (1H, m), 7.53-7.61 (1H, m), 8.35 (1H, s). MS m/z: 515 (M+1).

### Example 91

### Ethyl 5-cyano-6-(3-{[(2,4-difluorobenzyl)sulfonyl]carbamoyl}azetidin-1-yl)-2-(fluoromethyl)nicotinate

Prepared according to Method A from 1-[3-cyano-5-(ethoxycarbonyl)-6-(fluoromethyl)pyridin-2-yl]azetidine-3-carboxylic acid and 1-(2,4-difluorophenyl)methanesulfonamide to give ethyl 5-cyano-6-(3-{[(2,4-difluorobenzyl)sulfonyl]carbamoyl}azetidin-1-yl)-2-(fluoromethyl)nicotinate. Yield: 17.7 mg (36%).

¹H NMR (600 MHz, DMSO-d₆) δ 1.26 (39H, t, J=7.1 Hz), 3.54-3.60 (1H, m), 4.21 (2H, q, J=7.1 Hz), 4.29-4.52 (4H, m), 4.75 (2H, s), 5.64 (2H, d, J=47.8 Hz), 7.10-7.15 (1H, m), 7.24-7.30 (1H, m), 7.46-7.52 (1H, m), 8.36 (3H, s). MS m/z: 497 (M+1).

### Example 92

### Ethyl 6-(3-{[(4-chloro-2-fluorobenzyl)sulfonyl]carbamoyl}azetidin-1-yl)-5-cyano-2-(fluoromethyl)nicotinate

Prepared according to Method A from 1-[3-cyano-5-(ethoxycarbonyl)-6-(fluoromethyl)pyridin-2-yl]azetidine-3-carboxylic acid and 1-(4-chloro-2-fluorophenyl)methanesulfonamide to give ethyl 6-(3-{[(4-chloro-2-fluorobenzyl)sulfonyl]carbamoyl}azetidin-1-yl)-5-cyano-2-(fluoromethyl)nicotinate. Yield: 19.9 mg (39%).

¹H NMR (600 MHz, DMSO-d₆) δ 1.27 (3H, t, J=6.9 Hz), 3.54-3.61 (1H, m), 4.21 (2H, q, J=6.8 Hz), 4.29-4.52 (4H, m), 4.77 (2H, s), 5.64 (2H, d, J=47.4 Hz), 7.32-7.35 (1H, m), 7.44-7.50 (2H, m), 8.36 (1H, s). MS m/z: 513 (M+1).

### Example 93

### Ethyl 5-cyano-6-(3-{[(2,6-difluorobenzyl)sulfonyl]carbamoyl}azetidin-1-yl)-2-(difluoromethyl)nicotinate

Prepared according to Method A from 1-[3-cyano-6-(difluoromethyl)-5-(ethoxycarbonyl)pyridin-2-yl]azetidine-3-carboxylic acid and 1-(2,6-difluorophenyl)methanesulfonamide to give ethyl 5-cyano-6-(3-{[(2,6-difluorobenzyl)sulfonyl]carbamoyl}azetidin-1-yl)-2-(difluoromethyl)nicotinate. Yield: 14.5 mg (28%).

¹H NMR (600 MHz, DMSO-d₆) δ 1.28 (3H, t, J=7.1 Hz), 3.53-3.61 (1H, m), 4.24 (2H, q, J=7.1 Hz), 4.30-4.56 (4H, m), 4.75 (1H, s), 7.10-7.17 (2H, m), 7.37 (1H, t, J=54.2 Hz), 7.44-7.53 (1H, m), 8.44 (1H, s). MS m/z: 515 (M+1).

### Example 94

### Ethyl 5-cyano-2-(difluoromethyl)-6-(3-{[(4-fluoro-3-methylbenzyl)sulfonyl]carbamoyl}azetidin-1-yl)nicotinate

Prepared according to Method A from 1-[3-cyano-6-(difluoromethyl)-5-(ethoxycarbonyl)pyridin-2-yl]azetidine-3-carboxylic acid and 1-(4-fluoro-3-methylphenyl)methanesulfonamide to give ethyl 5-cyano-2-(difluoromethyl)-6-(3-{[(4-fluoro-3-methylbenzyl)sulfonyl]carbamoyl}azetidin-1-yl)nicotinate. Yield: 24.7 mg (48%).

¹H NMR (600 MHz, DMSO-d₆) δ 1.28 (3H, t, J=7.1 Hz), 2.17 (3H, s), 3.52-3.59 (1H, m), 4.25 (2H, q, J=7.1 Hz), 4.27-4.50 (4H, m), 4.67 (2H, s), 7.08-7.13 (1H, m), 7.16-7.22 (2H, m), 7.37 (1H, t, J=54.8 Hz), 8.45 (1H, s). MS m/z: 511 (M+1).

### Example 95

### Ethyl 6-(3-{[(2-chloro-4-fluorobenzyl)sulfonyl]carbamoyl}azetidin-1-yl)-5-cyano-2-(difluoromethyl)nicotinate

Prepared according to Method A from 1-[3-cyano-6-(difluoromethyl)-5-(ethoxycarbonyl)pyridin-2-yl]azetidine-3-carboxylic acid and 1-(2-chloro-4-fluorophenyl)methanesulfonamide to give Ethyl 6-(3-{[(2-chloro-4-fluorobenzyl)sulfonyl]carbamoyl}azetidin-1-yl)-5-cyano-2-(difluoromethyl)nicotinate. Yield: 24.6 mg (46%).

¹H NMR (600 MHz, DMSO-d₆) δ 1.26 (3H, t, J=7.2 Hz), 3.56-3.62 (1H, m), 4.23 (2H, q, J=7.2 Hz), 4.29-4.54 (4H, m), 4.85 (2H, s), 7.23-7.29 (1H, m), 7.36 (1H, t, J=52.7 Hz), 7.43-7.56 (2H, m), 8.43 (1H, s). MS m/z: 531 (M+1).

### Example 96

### Ethyl 5-cyano-2-(difluoromethyl)-6-(3-{[(5-fluoro-2-methylbenzyl)sulfonyl]carbamoyl}azetidin-1-yl)nicotinate

Prepared according to Method A from 1-[3-cyano-6-(difluoromethyl)-5-(ethoxycarbonyl)pyridin-2-yl]azetidine-3-carboxylic acid and 1-(5-fluoro-2-methylphenyl)methanesulfonamide to give ethyl 5-cyano-2-(difluoromethyl)-6-(3-{[(5-fluoro-2-methylbenzyl)sulfonyl]carbamoyl}azetidin-1-yl)nicotinate. Yield: 30.8 mg (60%).

¹H NMR (600 MHz, DMSO-d₆) δ 1.26 (3H, t, J=6.9 Hz), 2.30 (3H, s), 3.57-3.63 (1H, m), 4.23 (2H, q, J=7.4 Hz), 4.29-4.54 (4H, m), 4.75 (2H, s), 7.02-7.12 (2H, m), 7.22-7.27 (1H, m), 7.35 (1H, t, J=53.9 Hz), 8.43 (1H, s). MS m/z: 511 (M+1).

### Example 97

### Ethyl 5-cyano-6-(3-{[(2,4-difluorobenzyl)sulfonyl]carbamoyl}azetidin-1-yl)-2-(difluoromethyl)nicotinate

Prepared according to Method A from 1-[3-cyano-6-(difluoromethyl)-5-(ethoxycarbonyl)pyridin-2-yl]azetidine-3-carboxylic acid and 1-(2,4-difluorophenyl)methanesulfonamide to give ethyl 5-cyano-6-(3-{[(2,4-difluorobenzyl)sulfonyl]carbamoyl}azetidin-1-yl)-2-(difluoromethyl)nicotinate. Yield: 24.2 mg (47%).

¹H NMR (600 MHz, DMSO-d₆) δ 1.26 (3H, t, J=7.0 Hz), 3.54-3.61 (1H, m), 4.23 (2H, q, J=7.1 Hz), 4.30-4.53 (4H, m), 4.75 (2H, s), 7.09-7.13 (1H, m), 7.22-7.27 (1H, m), 7.36 (1H, t, J=54.0 Hz), 7.46-7.51 (1H, m), 8.43 (1H, s). MS m/z: 515 (M+1).

### Example 98

### Ethyl 6-(3-{[(4-chloro-2-fluorobenzyl)sulfonyl]carbamoyl}azetidin-1-yl)-5-cyano-2-(difluoromethyl)nicotinate

Prepared according to Method A from 1-[3-cyano-6-(difluoromethyl)-5-(ethoxycarbonyl)pyridin-2-yl]azetidine-3-carboxylic acid and 1-(4-chloro-2-fluorophenyl)methanesulfonamide to give ethyl 6-(3-{[(4-chloro-2-fluorobenzyl)sulfonyl]carbamoyl}azetidin-1-yl)-5-cyano-2-(difluoromethyl)nicotinate. Yield: 27 mg (51%).

¹H NMR (600 MHz, DMSO-d₆) δ 1.28 (3H, t, J=7.0 Hz), 3.55-3.62 (1H, m), 4.25 (2H, q, J=7.4 Hz), 4.29-4.56 (4H, m), 4.77 (2H, s), 7.31-7.35 (1H, m), 7.39 (1H, t, J=59.6 Hz), 7.45-7.49 (2H, m), 8.45 (1H, s). MS m/z: 531 (M+1).

### Example 99

### Ethyl 5-cyano-6-(3-{[(2,6-difluorobenzyl)sulfonyl]carbamoyl}azetidin-1-yl)-2-(trifluoromethyl)nicotinate

Prepared according to Method A from 1-[3-cyano-6-(trifluoromethyl)-5-(ethoxycarbonyl)pyridin-2-yl]azetidine-3-carboxylic acid and 1-(2,6-difluorophenyl)methanesulfonamide to give ethyl 5-cyano-6-(3-{[(2,6-difluorobenzyl)sulfonyl]carbamoyl}azetidin-1-yl)-2-(trifluoromethyl)nicotinate. Yield: 14.4 mg (27%).

¹H NMR (600 MHz, DMSO-d₆) δ 1.25 (3H, t, J=7.1 Hz), 3.54-3.61 (1H, m), 4.24 (2H, q, J=7.3 Hz), 4.30-4.54 (4H, m), 4.75 (2H, s), 7.11-7.17 (2H, m), 7.46-7.53 (1H, m), 8.47 (1H, s). MS m/z: 533 (M+1).

### Example 100

### Ethyl 5-cyano-6-(3-{[(4-fluoro-3-methylbenzyl)sulfonyl]carbamoyl}azetidin-1-yl)-2-(trifluoromethyl)nicotinate

Prepared according to Method A from 1-[3-cyano-6-(trifluoromethyl)-5-(ethoxycarbonyl)pyridin-2-yl]azetidine-3-carboxylic acid and 1-(4-fluoro-3-methylphenyl)methanesulfonamide to give ethyl 5-cyano-6-(3-{[(4-fluoro-3-methylbenzyl)sulfonyl]carbamoyl}azetidin-1-yl)-2-(trifluoromethyl)nicotinate. Yield: 26.2 mg (49%).

¹H NMR (600 MHz, DMSO-d₆) δ 1.24 (3H, t, J=7.5 Hz), 2.15 (3H, s), 3.50-3.57 (1H, m), 4.21-4.47 (4H, m), 4.23 (2H, q, J=7.4 Hz), 4.64 (2H, s), 7.04-7.23 (3H, m), 8.46 (1H, s). MS m/z: 529 (M+1).

### Example 101

### Ethyl 6-(3-{[(2-chloro-4-fluorobenzyl)sulfonyl]carbamoyl}azetidin-1-yl)-5-cyano-2-(trifluoromethyl)nicotinate

Prepared according to Method A from 1-[3-cyano-6-(trifluoromethyl)-5-(ethoxycarbonyl)pyridin-2-yl]azetidine-3-carboxylic acid and 1-(2-chloro-4-fluorophenyl)methanesulfonamide to give Ethyl 6-(3-{[(2-chloro-4-fluorobenzyl)sulfonyl]carbamoyl}azetidin-1-yl)-5-cyano-2-(trifluoromethyl)nicotinate. Yield: 34.5 mg (63%).

¹H NMR (600 MHz, DMSO-d₆) δ 1.24 (3H, t, J=7.3 Hz), 3.54-3.62 (1H, m), 4.23 (2H, q, J=7.3 Hz), 4.28-4.53 (4H, m), 4.83 (2H, s), 7.23-7.28 (1H, m), 7.45-7.56 (2H, m), 8.46 (1H, s). MS m/z: 549 (M+1).

### Example 102

### Ethyl 5-cyano-6-(3-{[(5-fluoro-2-methylbenzyl)sulfonyl]carbamoyl}azetidin-1-yl)-2-(trifluoromethyl)nicotinate

Prepared according to Method A from 1-[3-cyano-6-(trifluoromethyl)-5-(ethoxycarbonyl)pyridin-2-yl]azetidine-3-carboxylic acid and 1-(5-fluoro-2-methylphenyl)methanesulfonamide to give Ethyl 5-cyano-6-(3-{[(5-fluoro-2-methylbenzyl)sulfonyl]carbamoyl}azetidin-1-yl)-2-(trifluoromethyl)nicotinate. Yield: 36.6 mg (69%).

¹H NMR (600 MHz, DMSO-d₆) δ 1.24 (3H, t, J=7.4 Hz), 2.30 (3H, s), 3.57-3.63 (1H, m), 4.23 (2H, q, J=7.4 Hz), 4.27-4.53 (4H, m), 4.75 (2H, s), 7.02-7.12 (2H, m), 7.22-7.27 (1H, m), 8.46 (1H, s). MS m/z: 529 (M+1).

### Example 103

### Ethyl 5-cyano-6-(3-{[(2,3,6-trifluorobenzyl)sulfonyl]carbamoyl}azetidin-1-yl)-2-(trifluoromethyl)nicotinate

Prepared according to Method A from 1-[3-cyano-6-(trifluoromethyl)-5-(ethoxycarbonyl)pyridin-2-yl]azetidine-3-carboxylic acid and 1-(2,3,6-trifluorophenyl)methanesulfonamide to give Ethyl 5-cyano-6-(3-{[(2,3,6-trifluorobenzyl)sulfonyl]carbamoyl}azetidin-1-yl)-2-(trifluoromethyl)nicotinate. Yield: 31.3 mg (57%).

¹H NMR (600 MHz, DMSO-d₆) δ 1.24 (3H, t, J=7.0 Hz), 3.56-3.62 (1H, m), 4.23 (2H, q, J=7.2 Hz), 4.28-4.54 (4H, m), 4.80 (2H, s), 7.17-7.22 (1H, m), 7.54-7.60 (1H, m), 8.46 (1H, s). MS m/z: 551 (M+1).

### Example 104

### Ethyl 6-(3-{[(4-chloro-2-fluorobenzyl)sulfonyl]carbamoyl}azetidin-1-yl)-5-cyano-2-(trifluoromethyl)nicotinate

Prepared according to Method A from 1-[3-cyano-6-(trifluoromethyl)-5-(ethoxycarbonyl)pyridin-2-yl]azetidine-3-carboxylic acid and 1-(4-chloro-2-fluorophenyl)methanesulfonamide to give Ethyl 6-(3-{[(4-chloro-2-fluorobenzyl)sulfonyl]carbamoyl}azetidin-1-yl)-5-cyano-2-(trifluoromethyl)nicotinate. Yield: 27.2 mg (49%).

¹H NMR (600 MHz, DMSO-d₆) δ 1.24 (3H, t, J=7.4 Hz), 3.53-3.60 (1H, m), 4.23 (2H, q, J=7.2 Hz), 4.27-4.54 (4H, m), 4.75 (2H, s), 7.28-7.33 (1H, m), 7.41-7.48 (2H, m), 8.46 (1H, s). MS m/z: 549 (M+1).

### Example 105

### Ethyl 5-cyano-6-(4-{[(2,6-difluorobenzyl)sulfonyl]carbamoyl}piperidin-1-yl)-2-(difluoromethyl)nicotinate

Prepared according to Method A from 1-[3-cyano-6-(difluoromethyl)-5-(ethoxycarbonyl)pyridin-2-yl]piperidine-4-carboxylic acid and 1-(2,6-difluorophenyl)methanesulfonamide to give Ethyl 5-cyano-6-(4-{[(2,6-difluorobenzyl)sulfonyl]carbamoyl}piperidin-1-yl)-2-(difluoromethyl)nicotinate. Yield: 7.8 mg (14%).

¹H NMR (600 MHz, DMSO-d₆) δ 1.25 (3H, t, J=18.3 Hz), 1.59-1.66 (2H, m), 1.86-1.90 (2H, m), 3.17-3.23 (2H, m), 4.24 (2H, q, J=7.4 Hz), 4.52-4.57 (4H, m), 4.70 (2H, s), 7.12-7.18 (2H, m), 7.35 (1H, t, J=54.2 Hz), 7.44-7.51 (1H, m), 8.45 (1H, s). Note: One H signal overlaps with the DMSO signal. MS m/z: 543 (M+1).

### Example 106

### Ethyl 5-cyano-2-(difluoromethyl)-6-(4-{[(4-fluoro-3-methylbenzyl)sulfonyl]carbamoyl}piperidin-1-yl)nicotinate

Prepared according to Method A from 1-[3-cyano-6-(difluoromethyl)-5-(ethoxycarbonyl)pyridin-2-yl]piperidine-4-carboxylic acid and 1-(4-fluoro-3-methylphenyl)methanesulfonamide to give Ethyl 5-cyano-2-(difluoromethyl)-6-(4-{[(4-fluoro-3-methylbenzyl)sulfonyl]carbamoyl}piperidin-1-yl)nicotinate. Yield: 29.1 mg (54%).

¹H NMR (600 MHz, DMSO-d₆) [symbol] 1.27 (3H, t, J=7.0 Hz), 1.57-1.66 (3H, m), 1.78-1.83 (2H, m), 2.17 (3H, s), 3.14-3.21 (2H, m), 4.24 (2H, q, J=7.0 Hz), 4.50-4.55 (2H, m), 4.60 (2H, s), 7.08-7.15 (3H, m), 7.35 (1H, t, J=53.9 Hz), 8.46 (1H, s). Note: One H signal overlaps with the DMSO signal. MS m/z: 539 (M+1).

### Example 107

### Ethyl 5-cyano-2-(fluoromethyl)-6-(3-{[(2-fluoro-5-methylbenzyl)sulfonyl]carbamoyl}azetidin-1-yl)nicotinate

DIPEA (452 mg, 0.5 mmol) and TBTU (339 mg, 0.15 mmol) dissolved in DCM/DMF (1 mL, 1/1) was added to a solution of 1-[3-cyano-5-(ethoxycarbonyl)-6-(fluoromethyl)pyridin-2-yl]azetidine-3-carboxylic acid (31.1 mg, 0.1 mmol) in DCM/DMF (2 mL, 1/1) and the mixture was stirred at r.t for 20 minutes followed by addition of 1-(2-fluoro-5-methylphenyl)methanesulfonamide (149.2 mg, 0.1 mmol) dissolved in DCM/DMF (1 mL, 1/1). The mixture was stirred over night at r.t. LC-MS indicated that some starting material was left and therefore additional DIPEA (452 mg, 0.5 mmol) and DMAP (2.44 mg, 0.02 mmol) was added. The stirring was continued for 2 days but LC-MS still indicated some unreacted starting material. Addition of PyBrop (46.6 mg, 0.1 mmol) followed by stirring over night led to complete conversion to the product. The solvent was evaporated and the crude product was purified by preparative HPLC using the same procedure as described in Method A (See General Experimental Procedure). Yield: 21.6 mg (44%).

¹H NMR (400 MHz, DMSO-d₆): [symbol] 1.29 (3H, t, J=7.0 Hz), 2.27 (3H, s), 3.54-3.64 (1H, m), 4.24 (2H, q, J=7.0 Hz), 4.33-4.54 (4H, m), 4.72 (2H, s), 5.67 (2H, d, J=47.3 Hz), 7.08-7.15 (1H, m), 7.18-7.26 (2H, m), 8.38 (1H, s), 11.93 (1H, br s). MS m/z: 493 (M+1), 491 (M−1).

### Example 108

### Ethyl 5-cyano-6-(4-{[(2-fluoro-5-methylbenzyl)sulfonyl]carbamoyl}piperidin-1-yl)-2-(trifluoromethyl)nicotinate

Prepared according to the procedure described in Example 107 from 1-[3-cyano-5-(ethoxycarbonyl)-6-(trifluoromethyl)pyridin-2-yl]piperidine-4-carboxylic acid and 1-(2-fluoro-5-methylphenyl)methanesulfonamide. Yield: 3.9 mg (7%).

¹H NMR (400 MHz, DMSO-d₆): [symbol] 1.28 (3H, t, J=7.0 Hz), 1.61-1.74 (2H, m), 1.84-1.92 (2H, m), 2.26 (3H, s), 2.54-2.62 (1H, m), 3.20-3.29 (2H, m), 4.28 (2H, q, J=7.0 Hz), 4.46-4.54 (2H, m), 4.59 (2H, s), 7.06-7.23 (3H, m), 8.53 (1H, s), 11.73 (1H, br s). MS m/z: 557 (M+1), 555 (M−1).

### Example 109

### Ethyl 5-cyano-6-(3-{[(2-fluoro-5-methylbenzyl)sulfonyl]carbamoyl}azetidin-1-yl)-2-(trifluoromethyl)nicotinate

Prepared according to the procedure described in Example 107 from 1-[3-cyano-5-(ethoxycarbonyl)-6-(trifluoromethyl)pyridin-2-yl]azetidine-3-carboxylic acid and 1-(2-fluoro-5-methylphenyl)methanesulfonamide. Yield: 16.1 mg (30%).

¹H NMR (400 MHz, DMSO-d₆): [symbol] 1.27 (3H, t, J=7.0 Hz), 2.23 (3H, s), 3.38-3.50 (1H, m), 4.26 (2H, q, J=7.0 Hz), 4.30-4.49 (4H, m), 4.52 (2H, s), 7.00-7.09 (1H, m), 7.11-7.21 (2H, m), 8.47 (1H, s), 11.93 (1H, br s). MS m/z: 529 (M+1), 527 (M−1).

### Example 110

### Ethyl 5-cyano-2-(difluoromethyl)-6-(4-{[(2-fluoro-5-methylbenzyl)sulfonyl]carbamoyl}piperidin-1-yl)nicotinate

Prepared according to the procedure described in Example 107 from 1-[3-cyano-6-(difluoromethyl)-5-(ethoxycarbonyl)pyridin-2-yl]piperidine-4-carboxylic acid and 1-(2-fluoro-5-methylphenyl)methanesulfonamide. Yield: 9.9 mg (18%).

¹H NMR (400 MHz, DMSO-d₆): δ 1.31 (3H, t, J=7.0 Hz), 1.60-1.73 (2H, m), 1.84-1.92 (2H, m), 2.27 (3H, s), 2.54-2.63 (1H, m), 3.20-3.29 (2H, m), 4.28 (2H, q, J=7.0 Hz), 4.52-4.61 (2H, m), 4.63 (2H, s), 7.08-7.25 (3H, m), 7.39 (1H, t, J=54.0 Hz), 8.49 (1H, s), 11.73 (1H, br s). MS m/z: 539 (M+1), 537 (M−1).

### Example 111

### Ethyl 5-cyano-2-(difluoromethyl)-6-(3-{[(3-methoxybenzyl)sulfonyl]carbamoyl}azetidin-1-yl)nicotinate

1-[3-cyano-6-(difluoromethyl)-5-(ethoxycarbonyl)pyridin-2-yl]azetidine-3-carboxylic acid (135 mg, 0.41 mmol) and TBTU (176 mg, 0.55 mmol), were mixed in dry DCM (4 mL) and DIPEA (0.3 mL, 1.72 mmol) was added. The reaction mixture was stirred at r.t for 1.5 h and 1-(3-methoxyphenyl)methanesulfonamide (113 mg, 0.56 mmol) was added. The reaction mixture was stirred at r.t for 18 h. NaHCO₃ (aq) was added and the mixture was extracted with DCM (×3). The combined organic layer was run through a phase separator and evaporated. The crude product was purified by preparative HPLC (Kromasil C₈, 10 μm, 21.5×250 mm column, eluent A: 100% acetonitrile, eluent B: 0.1 M NH₄OAc in water containing 5% acetonitrile, flow 25 mL/min, using a gradient of 20-40% eluent A over 35 minutes) to give ethyl 5-cyano-2-(difluoromethyl)-6-(3-{[(3-methoxybenzyl)sulfonyl]carbamoyl}azetidin-1-yl)nicotinate as a white solid. Yield: 111 mg (53%). MS m/z: 509 (M+1), 507 (M−1).

### Example 112

### Ethyl 6-{4-[(benzylsulfonyl)carbamoyl]piperidin-1-yl}-5-cyano-2-(pentafluoroethyl)nicotinate

### (a) Ethyl-2-[(dimethylamino)methylene]-4,4,5,5,5-pentafluoro-3-oxopentanoate

Prepared in essentially the same way as described in Example 2(a) from 1,1-Dimethoxy-N,N-dimethylmethaneamine and ethyl 4,4,5,5,5-pentafluoro-3-oxopentanoate to give the product.

### (b) Ethyl 5-cyano-6-oxo-2-(pentafluoroethyl)-1,6-dihydropyridine-3-carboxylate

Cyanoacetamide (345 mg, 4.10 mmol) was suspensioned in EtOH (10 mL) and NaOEt (1.55 mL, 21% in EtOH, 4.15 mmol) was added dropwise and the mixture was stirred at rt for 30 min. Ethyl-2-[(dimethylamino)methylene]-4,4,5,5,5-pentafluoro-3-oxopentanoate (1.08 g, 3.73 mmol) dissolved in EtOH (5 mL) was added and the reaction mixture was stirred at rt over night. AcOH (0.5 mL) was added and solvent was evaporated. Water was added and the mixture was extracted with DCM (×3). The combined organic layer was run through a phase separator and evaporated. The crude product was purified by preparative HPLC (Kromasil C₈, 10 μm, 50.8×300 mm column, eluent A: 100% acetonitrile, eluent B: 0.1M NH₄OAc in water containing 5% acetonitrile, flow 50 mL/min, using a gradient of 10-40% eluent A over 60 minutes) to give Ethyl 5-cyano-6-oxo-2-(pentafluoroethyl)-1,6-dihydropyridine-3-carboxylate as a solid. Yield: 243 mg (21%). MS m/z: 309 (M−1).

### (c) Ethyl 6-chloro-5-cyano-2-(pentafluoroethyl)nicotinate

Ethyl 5-cyano-6-oxo-2-(pentafluoroethyl)-1,6-dihydropyridine-3-carboxylate (240 mg, 0.77 mmol) was suspensioned in toluene (30 mL) and SOCl₂ (0.5 mL, 6.9 mmol) and DMF (0.1 mL, 1.3 mmol) were added drop wise. The reaction mixture was heated to 80° C. for 20 h. Solvents was evaporated and the crude (440 mg) was used in the next step without further purification.

### (d) Ethyl 6-{4-[(benzylsulfonyl)carbamoyl]piperidin-1-yl}-5-cyano-2-(pentafluoroethyl)nicotinate

The crude ethyl 6-chloro-5-cyano-2-(pentafluoroethyl)nicotinate (100 mg, 0.30 mmol), N-(benzylsulfonyl)piperidine-4-carboxamide (96 mg, 0.34 mmol) and DIPEA (0.3 mL, 1.72 mmol) were mixed in EtOH (4 mL) and the reaction mixture was heated to 120° C. for 5 min in a microwave oven. NaHCO₃(aq) was added and the mixture was extracted with DCM (×3). The combined organic layer was run through a phase separator and evaporated. The crude product was purified by preparative HPLC (Kromasil C₈ 10 μm, 21.5×250 mm column, eluent A: 100% acetonitrile, eluent B: 0.1M NH₄OAc in water containing 5% acetonitrile, flow 25 mL/min, using a gradient of 30-60% eluent A over 35 minutes) to give Ethyl 6-{4-[(benzylsulfonyl)carbamoyl]piperidin-1-yl}-5-cyano-2-(pentafluoroethyl)nicotinate as a solid. Yield: 108 mg (62%).

¹H NMR (500 MHz, DMSO-d₆): δ 1.29 (3H, t, J=7.1 Hz), 1.61-1.71 (2H, m), 1.82-1.88 (2H, m), 2.58-2.65 (1H, m), 3.20-3.27 (2H, m), 4.30 (2H, q, J=7.1 Hz), 4.42-4.48 (2H, m), 4.70 (2H, s), 7.27-7.32 (2H, m), 7.37-7.42 (3H, m), 8.56 (1H, s), 11.61 (1H, br s). MS m/z: 575 (M+1), 573 (M−1).

### Example 113

### Ethyl 6-{3-[(benzylsulfonyl)carbamoyl]azetidin-1-yl}-5-cyano-2-(pentafluoroethyl)nicotinate

Prepared according to the procedure described in Example 112 (d) from ethyl 6-chloro-5-cyano-2-(pentafluoroethyl)nicotinate and N-(benzylsulfonyl)azetidine-3-carboxamide to give ethyl 6-{3-[(benzylsulfonyl)carbamoyl]azetidin-1-yl}-5-cyano-2-(pentafluoroethyl)nicotinate as a solid. Yield: 35 mg (21%).

¹H NMR (500 MHz, DMSO-d₆): δ 1.29 (3H, t, J=7.1 Hz), 3.53 (1H, m), 4.28 (2H, q, J=7.1 Hz), 4.28-4.36 (2H, m), 4.36-4.46 (2H, m), 4.68 (2H, s), 7.32-7.37 (5H, m), 8.50 (1H, s), 11.80 (1H, br s). MS m/z: 547 (M+1), 545 (M−1).

### Example 114

### Ethyl 6-{3-[(benzylsulfonyl)carbamoyl]azetidin-1-yl}-5-cyano-2-(1-fluoroethyl)nicotinate

### (a) Ethyl-2-[(dimethylamino)methylene]-4-fluoro-3-oxopentanoate

Ethyl 4-fluoro-3-oxopentanoate (2.28 g, 14.1 mmol) was dissolved in dimethoxymethyl-dimethyl-amine (2.0 mL, 15.1 mmol) and the mixture was stirred at rt for 18 h. LCMS showed complete conversion. The mixture was concentrated under reduced pressure and the crude was used in the next step without further purification. Yield assumed quantitative. MS m/z: 218 (M+1).

### (b) Ethyl 5-cyano-2-(1-fluoroethyl)-6-oxo-1,6-dihydropyridine-3-carboxylate

Cyanoacetamide (1.176 g, 14.0 mmol) was suspensioned in EtOH (40 mL) and NaOEt (5.5 mL, 21% wt in EtOH, 14.7 mmol) was added. The reaction mixture was stirred at rt for 2 h. The crude ethyl-2-[(dimethylamino)methylene]-4-fluoro-3-oxopentanoate (3.04 g, 14.0 mmol) dissolved in EtOH (10 mL) was added and the reaction mixture was stirred at rt for 21 h. AcOH (1.5 mL) was added and solvent was evaporated. Water was added, the solid was filtered off and washed with water and dried under reduced pressure to give ethyl 5-cyano-2-(1-fluoroethyl)-6-oxo-1,6-dihydropyridine-3-carboxylate as a solid. Yield: 2.78 g (84%). MS m/z: 239 (M+1), 237 (M−1).

### (c) Ethyl 6-chloro-5-cyano-2-(1-fluoroethyl)nicotinate

Ethyl 5-cyano-2-(1-fluoroethyl)-6-oxo-1,6-dihydropyridine-3-carboxylate (1.026 g, 4.31 mmol) was suspensioned in toluene (45 mL), SOCl₂ (2.5 mL, 34.4 mmol) and dry DMF (0.3 mL, 3.87 mmol) were added. The reaction mixture was heated to 80° C. for 3 h. LCMS showed 28% starting material left. SOCl₂ (2 mL, 27.5 mmol) and DMF (0.3 mL, 3.87 mmol) were added and the reaction mixture was heated to 80° C. for 17 h. LCMS showed no starting material left. Solvents was evaporated and the crude product was used in the next step without further purification. Yield assumed quantitative. MS m/z: 257 (M+1), 255 (M−1).

### (d) Ethyl 6-{3-[(benzylsulfonyl)carbamoyl]azetidin-1-yl}-5-cyano-2-(1-fluoroethyl)nicotinate

The crude ethyl 6-chloro-5-cyano-2-(1-fluoroethyl)nicotinate (87 mg, 0.34 mmol) and N-(benzylsulfonyl)azetidine-3-carboxamide (87 mg, 0.34 mmol) were dissolved in EtOH (3 mL) and DIPEA (1 mL, 5.7 mmol) was added. The reaction mixture was heated to 120° C. for 5 min in a microwave oven. NaHCO₃(aq) was added and the mixture was extracted with DCM (×3). The combined organic layer was run through a phase separator and evaporated. The crude product was purified by preparative HPLC (Kromasil C₈ 10 μm, 21.5×250 mm column, eluent A: 100% acetonitrile, eluent B: 0.1M NH₄OAc in water containing 5% acetonitrile, flow 25 mL/min, using a gradient of 20-40% eluent A over 35 minutes) to give ethyl 6-{3-[(benzylsulfonyl)carbamoyl]azetidin-1-yl}-5-cyano-2-(1-fluoroethyl)nicotinate as a white solid. Yield: 63 mg (39%). MS m/z: 475 (M+1), MS m/z 473 (M+1).

### Example 115

### Ethyl 6-{4-[(benzylsulfonyl)carbamoyl]piperidin-1-yl}-5-cyano-2-(1-fluoroethyl)nicotinate

Prepared according to the procedure described in Example 114 (d) from ethyl 6-chloro-5-cyano-2-(1-fluoroethyl)nicotinate and N-(benzylsulfonyl)azetidine-3-carboxamide to give ethyl 6-{4-[(benzylsulfonyl)carbamoyl]piperidin-1-yl}-5-cyano-2-(1-fluoroethyl)nicotinate as a white solid. Yield: 40 mg (26%). MS m/z: 503 (M+1), 501 (M−1).

### Example 116

### Ethyl 6-(4-{[(2-chloro-4-fluorobenzyl)sulfonyl]carbamoyl}piperidin-1-yl)-5-cyano-2-(fluoromethyl)nicotinate

DIPEA (64 mg, 0.5 mmol) was added to a solution of 1-[3-cyano-6-(fluoromethyl)-5-(ethoxycarbonyl)pyridin-2-yl]piperidine-4-carboxylic (33.5 mg, 0.1 mmol) and TBTU (160 mg, 0.5 mmol) in DCM and the mixture was stirred for 10 min at r.t before 1-(4-fluoro-2-chlorophenyl)methanesulfonamide (22 mg, 0.10 mmol) was added. The reaction was allowed to stir over night. The reaction mixture was washed with 0.1 M KHSO₄ and the organic phases passed through a phase separator and evaporated in a vacuum centrifuge. The crude product obtained was purified by HPLC (See General experimental procedure) to give ethyl 6-(4-{[(2-chloro-4-fluorobenzyl)sulfonyl]carbamoyl}piperidin-1-yl)-5-cyano-2-(fluoromethyl)nicotinate Yield: 19 mg (34%).

¹H NMR (500 MHz, DMSO-d₆): [symbol] 1.31 (3H, t, J=7.1 Hz), 1.61-1.71 (2H, m), 1.88-1.95 (2H, m), 2.60-2.67 (1H, m), 3.18-3.26 (2H, m), 4.26 (2H, q, J=7.1 Hz), 4.58-4.64 (2H, m), 4.83 (2H, s), 5.69 (2H, d, J=47 Hz), 7.29-7.35 (1H, m), 7.48-7.52 (1H, m), 7.53-7.57 (1H, m), 8.41 (1H, s), 11.82 (1H, br s). MS m/z: 541 (M+1).

### Example 117

### Ethyl 5-cyano-6-(4-{[(2,4-difluorobenzyl)sulfonyl]carbamoyl}piperidin-1-yl)-2-(fluoromethyl)nicotinate

Prepared according to the procedure described in Example 116 from 1-[3-cyano-6-(fluoromethyl)-5-(ethoxycarbonyl)pyridin-2-yl]piperidine-4-carboxylic and 1-(2,4-diofluorophenyl)methanesulfonamide to give ethyl 5-cyano-6-(4-{[(2,4-difluorobenzyl)sulfonyl]carbamoyl}piperidin-1-yl)-2-(fluoromethyl)nicotinate. Yield: 8.7 mg (17%).

¹H NMR (400 MHz, DMSO-d₆): [symbol] 1.31 (3H, t, J=7.1 Hz), 1.61-1.70 (2H, m), 1.87-1.93 (2H, m), 2.58-2.60 (1H, m), 3.18-3.26 (2H, m), 4.26 (2H, q, J=7.1 Hz), 4.58-4.64 (2H, m), 4.72 (2H, s), 5.69 (2H, d, J=47 Hz), 7.14-7.20 (1H, m), 7.30-7.36 (1H, m), 7.43-7.49 (1H, m), 8.41 (1H, s), 11.77 (1H, br s). MS m/z: 525 (M+1)

### Example 118

### Ethyl 6-{4-[(benzylsulfonyl)carbamoyl]piperidin-1-yl}-2-(chloromethyl)-5-cyanonicotinate

### (a) Ethyl 2-(chloromethyl)-5-cyano-6-oxo-1,6-dihydropyridine-3-carboxylate

A mixture of ethyl 4-chloro-3-oxobutanoate (10 g, 60.75 mmol), acetic anhydride (27.3 g, 267.3 mmol) and triethylorthoformate was heated at 120° C. (bath temperature) for 3 hours. The dark mixture was concentrated in vacuo and co-evaporated once with toluene (50 mL). Heptane (50 mL) was added to precipitate the product and then removed in vacuo. The crude material was dissolved in EtOH (50 mL). In a separate flask, sodium ethoxide (50 mL, 60.75 mmol, prepared by reaction of sodium with EtOH (50 mL)) was added dropwise to a cold (<5° C.) solution of 2-cyanoacetamide (5.11 g, 60.75 mmol) in EtOH (50 mL) and the mixture was stirred for 30 minutes after which the solution of the crude material from above was added over 10 minutes and the stirring was continued at r.t over night. The solid formed was isolated by filtration and washed with MTBE (50 mL). Drying of the solid gave ethyl 2-(chloromethyl)-5-cyano-6-oxo-1,6-dihydropyridine-3-carboxylate as a beige solid. Yield: 8.15 g (56%).

¹H NMR (500 MHz, DMSO-d₆) δ 1.27 (3H, t, J=7.0 Hz), 4.16 (2H, q, J=7.0 Hz), 4.75 (2H, s), 8.02 (1H, s).

### (b) Ethyl 6-chloro-2-(chloromethyl)-5-cyanonicotinate

DMF (0.076 g, 1.04 mmol) was added to a stirred slurry of ethyl 2-(chloromethyl)-5-cyano-6-oxo-1,6-dihydropyridine-3-carboxylate (1.00 g, 4.16 mmol) and oxalyl chloride (10.55 g, 83.11 mmol) at r.t (immediate gas evolution was observed). The mixture was heated to 70° C. for 4 hours and then at 50° C. over night. The mixture was diluted with butyronitrile and evaporated (twice with 20 mL) to remove excess oxalylchloride. The residue was partioned between butyronitrile (50 mL) and water (50 mL) and the water phase was acidified with concentrated HCl (0.5 mL) followed by addition of MgCl₂(aq) to aid phase separation. The organic phase was separated and washed with water (25 mL), 20% Na₂CO₃(aq) (0.5 mL), MgCl₂(aq) (10 mL) and dried (MgSO₄). The crude material was purified by chromatography on silica (Eluent: heptane/EtOAc, using a gradient of 90:10 to 40:60% to give the desired product as a coulorless solid. Yield: 2.56 g (61%).

¹H NMR (500 MHz, DMSO-d₆) δ 1.36 (3H, t, J=7.1 Hz), 4.38 (2H, q, J=7.1 Hz), 5.09 (2H, s), 8.90 (1H, s). MS m/z: 258 (M−1).

### (c) Ethyl 6-{4-[(benzylsulfonyl)carbamoyl]piperidin-1-yl}-2-(chloromethyl)-5-cyanonicotinate

A microwave vial was charged with ethyl 6-chloro-2-(chloromethyl)-5-cyanonicotinate (540 mg, 2.08 mmol), N-(benzylsulfonyl)piperidine-4-carboxamide (618 mg, 2.19 mmol) and TEA (527 mg, 5.21 mmol) and heated to 100° C. for 10 minutes using a microwave oven. The solvent was removed in vacuo and the residue was partioned between iPrOAc (20 mL) and aq HCl (40 μL 37% HCl in 15 mL water). The aqueous phase was separated and re-extracted with iPrOAc (10 mL). The combined organic phase was washed with aqueous MgCl₂ (10 mL), dried (MgSO₄) and evaporated to give the product which was used without further purification. Yield: 929 mg (88%).

¹H NMR (500 MHz, CDCl₃) δ 1.41 (3H, t, J=7.1 Hz), 1.75-1.94 (4H, m), 2.50 (1H, ddd, J=15.0, 10.8, 4.1 Hz), 3.19 (2H, dd, J=25.1, 2.3 Hz), 4.37 (2H, q, J=7.2 Hz), 4.63 (2H, s), 4.71 (2H, d, J=13.7 Hz), 4.98 (2H, s), 7.27-7.45 (5H, m), 8.41 (1H, s).

### Example 119

### Ethyl 5-cyano-2-(difluoromethyl)-6-(3-{[(2-fluoro-5-methylbenzyl)sulfonyl]carbamoyl}azetidin-1-yl)nicotinate

Prepared according to the procedure described in Example 107 from 1-[3-cyano-6-(difluoromethyl)-5-(ethoxycarbonyl)pyridin-2-yl]azetidine-3-carboxylic acid and 1-(2-fluoro-5-methylphenyl)methanesulfonamide. Yield: 15.9 mg (31%).

¹H NMR (400 MHz, DMSO-d₆): δ 1.30 (3H, t, J=7.0 Hz), 2.27 (3H, s), 3.55-3.65 (1H, m), 4.27 (2H, q, J=7.0 Hz), 4.33-4.55 (4H, m), 4.72 (2H, s), 7.07-7.14 (1H, m), 7.18-7.26 (2H, m), 7.40 (1H, t, J=53.9 Hz), 8.47 (1H, s), 11.93 (1H, br s). MS m/z: 511 (M+1), 509 (M−1).

### Example 120

### Ethyl 6-{3-[(benzylsulfonyl)carbamoyl]azetidin-1-yl}-2-(chloromethyl)-5-cyanonicotinate

A microwave vial was charged with 6-chloro-2-(chloromethyl)-5-cyanonicotinate (417 mg, 1.61 mmol), N-(benzylsulfonyl)azetidine-3-carboxamide (429 mg, 1.69 mmol), TEA (407 mg, 4.02 mmol) and EtOH (5 mL) and heated to 100° C. for 10 minutes. The mixture was diluted with DCM (25 mL), water (10 mL) and concentrated HCl (226 □L). The phases was separated and the organic phase dried (MgSO₄) and evaporated to give the desired product as a pale yellow solid. Yield: 590 mg (77%).

¹H NMR (500 MHz, DMSO-d₆) [symbol] 1.32 (3H, t, J=7.1 Hz), 3.55-3.63 (1H, m), 4.28 (2H, q, J=7.1 Hz), 4.31-4.53 (4H, m), 4.76 (2H, s), 4.95 (2H, s), 7.31-7.43 (5H, m), 8.42 (1H, s), 11.83 (1H, s).

### Example 121

### Ethyl 5-cyano-6-(3-{[(3,4-difluorobenzyl)sulfonyl]carbamoyl}azetidin-1-yl)-2-(difluoromethyl)nicotinate

1-(3,4-difluorophenyl)methanesulfonamide (25 mg, 0.12 mmol) was added to a mixture of 1-[3-cyano-6-(difluoromethyl)-5-(ethoxycarbonyl)pyridin-2-yl]azetidine-3-carboxylic acid (28.9 mg, 0.1 mmol), PyBrop (70 mg, 0.15 mmol) and DIPEA (129 mg, 1 mmol) in DCM and the mixture was stirred at r.t. over night. Addition of 0.5 M KHSO₄ (2 mL) and collection of the organic phase using a phase separator gave a crude product which was subjected to Waters Oasis MAX cartridges (2×500 mg, tetra alkyl Ammonium phase). Addition of the product-mixture on the column was done at pH ca 10 (titration with 0.1 M NaOH) followed by washing with additional 0.1 M NaOH (2 mL), 1/1 CH₃CN/H₂O (4.5 mL) and 100% CH₃CN eluted the phosphorus triamide byproduct from the PyBrop reagent. The product was then eluted with 90% CH₃CN and 2% Formic acid. Evaporation of the solvent afforded the product as a white solid which was further purified by preparative HPLC according to the method described in the General Experimental Procedure to give ethyl 5-cyano-6-(3-{[(3,4-difluorobenzyl)sulfonyl]carbamoyl}azetidin-1-yl)-2-(difluoromethyl)nicotinate. Yield: 29 mg (56%).

¹H NMR (400 MHz, DMSO-d₆): δ 1.31 (3H, t, J=7.1 Hz), 3.55-3.64 (1H, m), 4.28 (2H, q, J=7.1 Hz), 4.32-4.39 (2H, m), 4.43-4.52 (2H, m), 4.77 (2H, s), 7.19-7.24 (1H, m), 7.40 (1H, t, J=54 Hz), 7.41-7.48 (2H, m), 8.48 (1H, s), 11.90 (1H, br s). MS m/z: 515 (M+1).

### Example 122

### Ethyl 5-cyano-6-(4-{[(3,4-difluorobenzyl)sulfonyl]carbamoyl}piperidin-1-yl)-2-(difluoromethyl)nicotinate

Prepared according to Example 121 from 1-[3-cyano-6-(difluoromethyl)-5-(ethoxycarbonyl)pyridin-2-yl]piperidine-4-carboxylic acid and 1-(3,4-difluorophenyl)methanesulfonamide to give ethyl 5-cyano-6-(4-{[(3,4-difluorobenzyl)sulfonyl]carbamoyl}piperidin-1-yl)-2-(difluoromethyl)nicotinate. Yield: 7 mg (13%).

¹H NMR (400 MHz, DMSO-d₆): [symbol] 1.32 (3H, t, J=7.1 Hz), 1.60-1.71 (2H, m), 1.84-1.91 (2H, m), 2.57-2.66 (1H, m), 3.19-3.28 (2H, m), 3.29 (2H, q, J=7.1 Hz), 4.54-4.61 (2H, m), 4.73 (2H, s), 7.12-7.16 (1H, m), 7.34-7.40 (1H, m), 7.40 (1H, t, J=54 Hz), 7.45-7.53 (1H, m), 8.51 (1H, s), 11.69 (1H, br s). MS m/z: 543 (M+1).

### Example 123

### Ethyl 5-cyano-6-(4-{[(2,4-difluorobenzyl)sulfonyl]carbamoyl}piperidin-1-yl)-2-(difluoromethyl)nicotinate

Prepared according to Example 121 from 1-[3-cyano-6-(difluoromethyl)-5-(ethoxycarbonyl)pyridin-2-yl]piperidine-4-carboxylic acid and 1-(2,4-difluorophenyl)methanesulfonamide to give ethyl 5-cyano-6-(4-{[(2,4-difluorobenzyl)sulfonyl]carbamoyl}piperidin-1-yl)-2-(difluoromethyl)nicotinate. Yield: 15 mg (27%).

¹H NMR (400 MHz, DMSO-d₆): [symbol] 1.32 (3H, t, J=7.1 Hz), 1.62-1.73 (2H, m), 1.88-1.95 (2H, m), 2.59-2.65 (1H, m), 3.19-3.28 (2H, m), 4.29 (2H, q, J=7.1 Hz), 4.55-4.62 (2H, m), 4.74 (2H, s), 7.14-7.21 (1H, m), 7.30-7.37 (1H, m), 7.40 (1H, t, J=54 Hz), 7.43-7.50 (1H, m), 8.51 (1H, s), 11.77 (1H, br s). MS m/z: 543 (M+1).

### Example 124

### Ethyl 6-{4-[(benzylsulfonyl)carbamoyl]piperidin-1-yl}-5-cyano-2-(2-fluoroethoxy)nicotinate

### (a) tert-Butyl 4-[allyl(benzylsulfonyl)carbamoyl]piperidine-1-carboxylate

A mixture of tert-butyl 4-[(benzylsulfonyl)carbamoyl]piperidine-1-carboxylate (11.47 g, 30 mmol, See Example 1(d)), 3-bromoprop-1-ene (10.89 g, 90 mmol) and DIPEA (7.76 g, 60 mmol) in DMF (30 mL) was stirred at r.t for 21 hours. Water (75 mL) was added and the aqueous phase was extracted with heptane/DCM 4/1 (3×75 mL). The combined organic phase was dried (MgSO₄), filtered and evaporated to give the product which was used without further purification.

### (b) N-allyl-N-(benzylsulfonyl)piperidine-4-carboxamide trifluoroacetate

TFA/DCM 2/1 (30 mL) was added to a stirred solution of tert-butyl 4-[allyl(benzylsulfonyl)carbamoyl]piperidine-1-carboxylate (12.68 g, 30 mmol) in DCM (10 mL) at 0° C. (ice/water bath) and the stirring was continued for 5 minutes followed by 4 hours at r.t. The solvent was evaporated and the mixture was co-evaporated with DCM twice to give the product as a TFA salt which was used in the next step without further purification.

### (c) N-allyl-N-(benzylsulfonyl)-1-(2-cyanoethanimidoyl)piperidine-4-carboxamide

N-allyl-N-(benzylsulfonyl)piperidine-4-carboxamide trifluoroacetate (30 mmol) was added to a cold (ice/water bath temperature) solution of ethyl 2-cyanoethanimidoate (See McElvain, S. M.; Schroeder, J. P.; J. Am. Chem. Soc. 71, p. 40 (1949)) (15.14 g, 101.25 mmol, 75% pure) and DIPEA (23.26 g, 180 mmol) in EtOH (200 mL) and the mixture was stirred for 10 minutes followed by 16 hours at r.t. LC-MS showed complete conversion of the starting material. This solution was used in the next step as such.

### (d) Ethyl 6-{4-[allyl(benzylsulfonyl)carbamoyl]piperidin-1-yl}-5-cyano-2-oxo-1,2-dihydropyridine-3-carboxylate

Diethyl (ethoxymethylene)malonate (8.43 g, 39 mmol) was added to the solution from step (d) above and the reaction mixture was stirred for 18 hours at r.t. Evaporation of the solvent gave 32 g of a crude product. 8 g (¼) of this was taken out and purified by preparative HPLC (Kromasil C₈, 10 μm, Eluent: A: CH₃CN; B: 0.2% AcOH in water/CH₃CN 95/5; C: 0.1 M NH₄OAc/CH₃CN 95/5. Using A/B/C 5/0/95 during injection and then eluting with a gradient going from A/B/C 5/95/0 to 100/0/0) to give two fractions containing the product. Fraction 1: 308 mg (8% chemical yield, 100% purity according to LC-MS and Fraction 2: 853 mg (76% pure according to LC-MS).

¹H-NMR (400 MHz, CDCl₃): δ 1.40 (3H, t, J=7.2 Hz), 1.57-1.80 (4H, m), 2.60-2.70 (1H, m), 2.92-3.03 (2H, m), 4.11-4.16 (2H, m), 4.39/2H, q, J=7.2 Hz), 4.61 (2H, s), 4.64-4.72 (2H, m), 5.19-5.30 (2H, m), 6.62-5.75 (1H, m), 7.31-7.45 (5H, m), 8.24 (1H, s), 11.90 (1H, br s, NH).

### (e) Ethyl 6-{4-[allyl(benzylsulfonyl)carbamoyl]piperidin-1-yl}-5-cyano-2-(2-fluoroethoxy)nicotinate

1-fluoro-2-iodoethane (142 mg, 0.82 mmol) was added to a mixture of ethyl 6-{4-[allyl(benzylsulfonyl)carbamoyl]piperidin-1-yl}-5-cyano-2-oxo-1,2-dihydropyridine-3-carboxylate (100 mg, 0.164 mmol) and Ag₂CO₃ (136 mg, 0.492 mmol) in acetonitrile (20 mL) under a nitrogen atmosphere and the mixture was heated to reflux for 1.5 hours. An additional 1-fluoro-2-iodoethane (142 mg, 0.82 mmol) was added and the reflux was continued for another 1.5 hours. LC-MS showed still some remaining starting material but after addition of an additional 1-fluoro-2-iodoethane (142 mg, 0.82 mmol) and refluxing over night the reaction was complete. The solvent was removed in vacuo and the crude product was used without further purification in the next step assuming a quantitative yield.

### (f) Ethyl 6-{4-[(benzylsulfonyl)carbamoyl]piperidin-1-yl}-5-cyano-2-(2-fluoroethoxy)nicotinate

Sodium 4-methylbenzenesulfonate (79 mg, 0.445 mmol) and tetrakis(triphenylphosphine)palladium (190 mg, 0.165 mmol) were added to a solution of ethyl 6-{4-[allyl(benzylsulfonyl)carbamoyl]piperidin-1-yl}-5-cyano-2-(2-fluoroethoxy)nicotinate (107 mg, 0.165 mmol, the crude product from the step above) in DCM (10 mL) under an atmosphere of nitrogen. The mixture was stirred 1 h at r.t and the solvent was removed in vacuo. The residue was purified by preparative HPLC (Kromasil C₈, 10 μm, 21.2×250 mm column using a gradient of 30% to 95% CH₃CN/0.1 M NH₄Oac) to give ethyl 6-{4-[(benzylsulfonyl)carbamoyl]piperidin-1-yl}-5-cyano-2-(2-fluoroethoxy)nicotinate as a yellow solid after freeze drying. Yield: 33 mg (38% over two steps).

¹H NMR (400 MHz, DMSO-d₆) δ 1.26 (3H, t, J=7.1 Hz), 1.55-1.70 (2H, m), 1.75-1.88 (2H, m), 2.25-2.39 (1H, m), 3.10-3.22 (2H, m), 4.19 (2H, q, J=7.3 Hz), 4.44-4.53 (2H, m), 4.53-4.57 (1H, m), 4.58-4.65 (3H, m), 4.66-4.71 (1H, m), 4.78-4.82 (1H, m), 7.24-7.30 (2H, m), 7.32-7.40 (3H, m), 8.28 (1H, s). MS m/z: 519 (M+1).

### Example 125

### Ethyl 6-{3-[(benzylsulfonyl)carbamoyl]azetidin-1-yl}-5-cyano-2-[(2,2,2-trifluoroethoxy)methyl]nicotinate

A microwave vial was charged with ethyl 6-{3-[(benzylsulfonyl)carbamoyl]azetidin-1-yl}-2-(chloromethyl)-5-cyanonicotinate (25 mg, 0.052 mmol, see Example 120), cesium carbonate (34 mg, 0.10 mmol), sodium iodide (8 mg, 0.052 mmol), 2,2,2-trifluoroethanol (0.36 mL, 5.0 mmol) and the reaction mixture was heated to 100° C. for 15 min in a microwave oven. LCMS indicated clean conversion to the desired product. Solvents was removed under reduced pressure and the remaining residue was partitioned between DCM and water. The organic phase was separated, concentrated under reduced pressure. The crude product obtained was purified by HPLC (See General experimental procedure) to give ethyl 6-{3-[(benzylsulfonyl)carbamoyl]azetidin-1-yl}-5-cyano-2-[(2,2,2-trifluoroethoxy)methyl]nicotinate. Yield: 5.6 mg (18%).

¹H NMR (600 MHz, DMSO-d₆): δ 1.27 (3H, t, J=7.2 Hz), 3.48-3.57 (1H, m), 4.18 (2H, q, J=9.3 Hz), 4.21 (2H, q, J=7.2 Hz), 4.28-4.34 (2H, m), 4.38-4.46 (2H, m), 4.70 (2H, br s), 4.97 (2H, s), 7.28-7.36 (5H, m), 8.32 (1H, s). MS m/z: 541 (M+1).

### Example 126

### Ethyl 6-{4-[(benzylsulfonyl)carbamoyl]piperidin-1-yl}-5-cyano-2-[(2,2,2-trifluoroethoxy)methyl]nicotinate

Prepared according to the procedure in example 125 using ethyl 6-{4-[(benzylsulfonyl)carbamoyl]piperidin-1-yl}-2-(chloromethyl)-5-cyanonicotinate to give ethyl 6-{4-[(benzylsulfonyl)carbamoyl]piperidin-1-yl}-5-cyano-2-[(2,2,2-trifluoroethoxy)methyl]nicotinate. Yield: 7.3 mg (24%).

¹H NMR (600 MHz, DMSO-d₆): δ 1.27 (3H, t, J=7.1 Hz), 1.58-1.66 (2H, m), 1.78-1.85 (2H, m), 3.13-3.21 (2H, m), 4.17 (2H, q, J=9.1 Hz), 4.22 (2H, q, J=6.9 Hz), 4.52-4.58 (2H, m), 4.66 (2H, s), 4.98 (2H, s), 7.24-7.28 (2H, m), 7.33-7.39 (3H, m), 8.35 (1H, s). MS m/z: 569 (M+1).

### Example 127

### Ethyl 6-{4-[(benzylsulfonyl)carbamoyl]piperidin-1-yl}-5-cyano-2-(difluoromethoxy)nicotinate

### (a) Ethyl 6-{4-[allyl(benzylsulfonyl)carbamoyl]piperidin-1-yl}-5-cyano-2-(difluoromethoxy)nicotinate

In a microwave vial was placed ethyl 6-{4-[allyl(benzylsulfonyl)carbamoyl]piperidin-1-yl}-5-cyano-2-oxo-1,2-dihydropyridine-3-carboxylate (103 mg, 0.20 mmol, See Example 124(d)) dissolved in acetonitrile (2.5 mL) and 2-(fluorosulphonyl)difluoroacetic acid (0.062 mL, 0.60 mmol) was added. The reaction mixture was heated in a microwave oven to 80° C. for 5 min. LC/MS showed 46% product with right mass and 20% starting material. 2-(Fluorosulphonyl)difluoroacetic acid (0.124 mL, 1.20 mmol) was added and the reaction mixture was heated in a microwave oven to 100° C. for 5 min. LC/MS showed 46% product with right mass and 7% starting material. The reaction mixture was heated in a microwave oven to 100° C. for 15 min. LC/MS showed no change. The mixture was extracted with DCM (3×20 mL) and the combined organics was washed with 10% Na₂CO₃ (20 mL). Brine (around 5 mL) had to be added to obtain separation. The organic layer was washed with brine (50 mL), dried over anhydrous sodium sulphate and concentrated under reduced pressure to give 110 mg crude ethyl 6-{4-[allyl(benzylsulfonyl)carbamoyl]piperidin-1-yl}-5-cyano-2-(difluoromethoxy)nicotinate that was used in the next step without further purification. MS m/z: 563 (M+1).

### (b) Ethyl 6-{4-[(benzylsulfonyl)carbamoyl]piperidin-1-yl}-5-cyano-2-(difluoromethoxy)nicotinate

The crude ethyl 6-{4-[allyl(benzylsulfonyl)carbamoyl]piperidin-1-yl}-5-cyano-2-(difluoromethoxy)nicotinate (110 mg, 0.16 mmol, 80%) from previous step was dissolved in DCM (3 mL) and tetrakis(triphenylphosphine)palladium (18 mg, 0.016 mmol) was added followed by sodium 4-toluenesulphonate (59 mg, 0.33 mmol). The reaction mixture was stirred at rt under nitrogen for 20 h. LC/MS showed complete conversion. Solvents was removed under reduced pressure and the crude was purified by preparative HPLC (Kromasil C₈ 10 μm, 50.8×300 mm column, Eluent A: 100% acetonitrile, Eluent B: 0.2% acetic acid in water containing 5% acetonitrile, flow 75 mL/min, using a increasing gradient of acetonitrile over 17 minutes) to give ethyl 6-{4-[(benzylsulfonyl)carbamoyl]piperidin-1-yl}-5-cyano-2-(difluoromethoxy)nicotinate. Yield: 18 mg (22%).

¹H NMR (300 MHz, CDCl₃): δ 1.37 (3H, t, J=7.2 Hz), 1.71-1.95 (4H, m), 2.40-2.53 (1H, m), 3.12-3.26 (2H, m), 4.32 (2H, q, J=7.2 Hz), 4.48-4.59 (2H, m), 4.64 (2H, s), 7.29-7.42 (6H, m), 8.20-8.35 (1H, br s), 8.42 (1H, s). MS m/z: 523 (M+1).

### Example 128

### Ethyl 6-{4-[(benzylsulfonyl)carbamoyl]piperidin-1-yl}-5-cyano-2-(2,2-difluoroethoxy)nicotinate

### (a) Ethyl 6-{4-[allyl(benzylsulfonyl)carbamoyl]piperidin-1-yl}-5-cyano-2-(2,2-difluoroethoxy)nicotinate

Ethyl 6-{4-[allyl(benzylsulfonyl)carbamoyl]piperidin-1-yl}-5-cyano-2-oxo-1,2-dihydropyridine-3-carboxylate (100 mg, 0.16 mmol) was dissolved in dry DMSO (15 mL), Ag₂CO₃ (136 mg, 0.49 mmol) was added and the mixture was stirred at rt for 5 min under N₂. 2-Iodo-1,1-difluoroethane (629 mg, 3.28 mmol) was added and the reaction mixture was heated to 95° C. After 5 h another 5 eq of 2-Iodo-1,1-difluoroethane (157 mg, 0.82 mmol) was added, the temperature was decreased to 85° C. and the reaction mixture was stirred at 85° C. over night. Water was added and the mixture was extracted with DCM (×3). The combined organics was concentrated and the crude ethyl 6-{4-[allyl(benzylsulfonyl)carbamoyl]piperidin-1-yl}-5-cyano-2-(2,2-difluoroethoxy)nicotinate was used in the next step without further purification. MS m/z: 577 (M+1).

### (b) Ethyl 6-{4-[(benzylsulfonyl)carbamoyl]piperidin-1-yl}-5-cyano-2-(2,2-difluoroethoxy)nicotinate

Ethyl 6-{4-[allyl(benzylsulfonyl)carbamoyl]piperidin-1-yl}-5-cyano-2-(2,2-difluoroethoxy)nicotinate (94 mg, 0.16 mmol) was dissolved in dry DCM (10 mL), tetrakis(triphenylphosphine)palladium (188 mg, 0.16 mmol), sodium 4-toluenesulphonate (78 mg, 0.44 mmol) were added and the reaction mixture was stirred at r.t for 1.5 h. The mixture was filtered, concentrated under reduced pressure and the crude was purified by preparative HPLC (Kromasil C₈ 10 μm, 50.8×300 mm column, Eluent A: 100% acetonitrile, Eluent B: 0.2% acetic acid in water containing 5% acetonitrile, flow 50 mL/min, using a gradient of 30-100% acetonitrile over 35 minutes) to give ethyl 6-{4-[(benzylsulfonyl)carbamoyl]piperidin-1-yl}-5-cyano-2-(2,2-difluoroethoxy)nicotinate as a white solid. Yield: 5.9 mg (6.5%).

¹H NMR (400 MHz, DMSO-d₆): δ 1.26 (3H, t, J=7.3 Hz), 1.58-1.70 (2H, m), 1.79-1.87 (2H, m), 2.97-3.03 (1H, m), 3.13-3.22 (2H, m), 4.19 (2H, q, J=7.2 Hz), 4.46-4.54 (2H, m), 4.56-4.69 (4H, m), 6.38 (1H, t, J=52.6 Hz), 7.24-7.40 (5H, m), 8.32 (1H, s), 11.59 (1H, br s). MS m/z: 537 (M+1), 535 (M−1).

### Example 129

### Ethyl 6-{4-[(benzylsulfonyl)carbamoyl]piperidin-1-yl}-5-cyano-2-(2,2,2-trifluoroethoxy)nicotinate

### (a) Ethyl 6-{4-[allyl(benzylsulfonyl)carbamoyl]piperidin-1-yl}-5-cyano-2-{[(trifluoromethyl)sulfonyl]oxy}nicotinate

Ethyl 6-{4-[allyl(benzylsulfonyl)carbamoyl]piperidin-1-yl}-5-cyano-2-oxo-1,2-dihydropyridine-3-carboxylate (308 mg, 0.60 mmol) was dissolved in DCM (7 mL) and cooled to 0° C. under N₂. Triethylamine (0.37 mL, 2.7 mmol) was added followed by dropwise addition of trifluoromethanesulfonic anhydride. The reaction mixture was stirred at 0° C. for 1 h. NaHCO₃ (aq,sat) (10 mL) was added, the organic layer was separated and the aqueous layer was extracted with DCM (×2). The combined organics was dried over anhydrous sodium sulphate, filtered and concentrated under reduced pressure to give crude ethyl 6-{4-[allyl(benzylsulfonyl)carbamoyl]piperidin-1-yl}-5-cyano-2-{[(trifluoromethyl)sulfonyl]oxy}nicotinate that was used in the next step without further purification, assuming quantitative yield. MS m/z: 645 (M+1).

### (b) ethyl 6-{4-[(benzylsulfonyl)carbamoyl]piperidin-1-yl}-5-cyano-2-(2,2,2-trifluoroethoxy)nicotinate

Ethyl 6-{4-[allyl(benzylsulfonyl)carbamoyl]piperidin-1-yl}-5-cyano-2-{[(trifluoromethyl)sulfonyl]oxy}nicotinate (150 mg, 0.23 mmol), Pd₂(dba)₃ (21.3 mg, 0.023 mmol), Xantphos (13.5 mg, 0.023 mmol) were mixed in dioxane (3 mL), DIPEA (0.1 mL, 0.57 mmol) and 2,2,2-trifluoroethanol (100 mg, 1.0 mmol) were added. The reaction mixture was heated to 160° C. for 10 min in a microwave oven. LCMS showed complete conversion of starting material. NaHCO₃ (aq) was added and the mixture was extracted with DCM (×3). The combined organic layer was run through a phase separator and evaporated. The crude product was purified by preparative HPLC (Kromasil C₈ 10 μm, 21.5×250 mm column, eluent A: 100% acetonitrile, eluent B: 0.1 M NH₄OAc in water containing 5% acetonitrile, flow 25 mL/min, using a gradient of 20-55% eluent A over 35 minutes) to give ethyl 6-{4-[(benzylsulfonyl)carbamoyl]piperidin-1-yl}-5-cyano-2-(2,2,2-trifluoroethoxy)nicotinate as a solid. Yield: 24 mg (19%).

¹H NMR (500 MHz, DMSO-d₆) δ 1.27 (3H, t, J=7.1 Hz), 1.62-1.71 (2H, m), 1.82-1.88 (2H, m), 2.57-2.64 (1H, m), 3.16-3.23 (2H, m), 4.22 (2H, q, J=7.1 Hz), 4.53-4.59 (2H, m), 4.69 (2H, s), 5.05 (2H, q, J=8.8 Hz), 7.28-7.32 (2H, m), 7.38-7.42 (3H, m), 8.36 (1H, s), 11.61 (1H, br s). MS m/z: 555 (M+1), MS m/z: 553 (M−1).

### Example 130

### Ethyl 5-cyano-2-(difluoromethyl)-6-[3-({[4-(hydroxymethyl)benzyl]sulfonyl}carbamoyl)azetidin-1-yl]nicotinate

### (a) tert-Butyl {[4-(chloromethyl)benzyl]oxy}dimethylsilane

4-Chloro methyl benzyl alcohol (1.35 g, 8.6 mmol) and imidazol (763 mg, 11.2 mmol) was dissolved in CH₂Cl₂ and cooled to 0° C. and TBDMSCl (1.43 g, 9.5 mmol) was added in portions. A white precipitate was formed and the reaction mixture was stirred for 1 h. Water (30 mL) and 1M KHSO₄ (30 mL) was added and the mixture was stirred an additional 3 min. The organic layer was separated using a phase separator and evaporated to give tert-butyl {[4-(chloromethyl)benzyl]oxy}dimethylsilane as an oil, that was used without further purification. Yield: 2.4 g (103%).

### (b) Methyl 3-{[4-({[tert-butyl(dimethyl)silyl]oxy}methyl)benzyl]sulfonyl}propanoate

SMOPS (1.76 g, 10.1 mmol, Wang et. al. Tetrahedron Letters 43, 2002, 8479-8483) was dissolved in DMSO (20 mL) using a ultrazonic bath and was then added to tert-butyl{[4-(chloromethyl)benzyl]oxy}dimethylsilane (2.4 g, 8.4 mmol) dissolved in DMSO (5 mL), and the reaction mixture was stirred at rt over night. Water (30 mL) was added and the mixture was extracted twice with EtOAc. The combined organics was dried over anhydrous Na₂SO₄, filtered and evaporated. ¹H NMR indicated some DMSO left. To eliminate DMSO the crude product was dissolved in CH₂Cl₂ (40 mL), water (20 mL) was added and the two phase system was stirred for 30 min. The organic layer was separated using a phase separator and evaporated to give methyl 3-{[4-({[tert-butyl(dimethyl)silyl]oxy}methyl)benzyl]sulfonyl}propanoate as a solid. Yield: 3.1 g (95%). MS m/z: 404 (NH+adduct).

### (c) 1-[4-({[tert-Butyl(dimethyl)silyl]oxy}methyl)phenyl]methanesulfonamide

Methyl 3-{[4-({[tert-butyl(dimethyl)silyl]oxy}methyl)benzyl]sulfonyl}propanoate (3.1 g, 8.0 mmol) was dissolved in dry THF (20 mL) and a sodium methoxide solution, freshly prepared from sodium (221 mg, 9.6 mmol) in dry methanol (3 mL), was added at rt under nitrogen, LCMS check after 30 min revealed ca 10% starting material still present. Further sodium methoxide solution was added until the all starting material was consumed. To this reaction mixture was a water (30 mL) solution of hydroxylamine O-sulfonic acid (2.27 g, 20 mmol) and sodium acetate (2.5 g, 30 mmol) (acting as buffer) added and the reaction was stirred over night. Extraction with EtOAc (×2), drying over anhydrous Na₂SO₄, concentration and final removal of acetic acid using vacuum pump to give 1-[4-({[tert-butyl(dimethyl)silyl]oxy}methyl)phenyl]methanesulfonamide as a white solid. The crude product was used without further purification. Yield: 2.5 g (99%). MS m/z: 314 (M+1).

### (d) Ethyl 6-[3-({[4-({[tert-butyl(dimethyl)silyl]oxy}methyl)benzyl]sulfonyl}carbamoyl)azetidin-1-yl]-5-cyano-2-(difluoromethyl)nicotinate

1-[3-Cyano-6-(difluoromethyl)-5-(ethoxycarbonyl)pyridin-2-yl]azetidine-3-carboxylic acid (100 mg, 0.31 mmol, See example 9(a)) and 1-[4-({[tert-butyl(dimethyl)silyl]oxy}methyl)phenyl]methanesulfonamide (116 mg, 0.37 mmol) were charged together with PyBrop (215 mg, 0.46 mmol) in a glass flask (16 mL tube) when CH₂Cl₂ (4.5 mL) was added. To this stirred slurry was added DIPEA (0.54 mL, 3.1 mmol) and the reaction mixture turned into a clear solution. LCMS after 1 h showed complete conversion of starting material. Water was added, the organic layer was separated using a phase separator and concentrated on a vacuum centrifuge. The crude product was purified by preparative HPLC to give ethyl 6-[3-({[4-({[tert-butyl(dimethyl)silyl]oxy}methyl)benzyl]sulfonyl}carbamoyl)azetidin-1-yl]-5-cyano-2-(difluoromethyl)nicotinate as a white solid. Yield: 103 mg (48%).

### (e) Ethyl 5-cyano-2-(difluoromethyl)-6-[3-({[4-(hydroxymethyl)benzyl]sulfonyl}carbamoyl)azetidin-1-yl]nicotinate

Ethyl 6-[3-({[4-({[tert-butyl(dimethyl)silyl]oxy}methyl)benzyl]sulfonyl}carbamoyl)azetidin-1-yl]-5-cyano-2-(difluoromethyl)nicotinate (103 mg, 0.17 mmol) was dissolved in TFA at rt. LCMS after 15 min showed complete conversion of starting material to desired product and ca 15% TFA-ester. The reaction was unfortunately left over night resulting in full conversion to the TFA-ester. To the concentrated TFA-ester was NH₃(aq), 26% (1.5 mL) and CH₃CN (2 mL) added. After cleavage of the TFA-ester the mixture was evaporated on a vacuum centrifuge. Freeze drying from CH₃CN/H₂O gave a white powder. This crude solid containing NH₄TFA was dissolved in H₂O/CH₃CN and pH adjusted to ca 10 with 0.1M NaOH. The solution was charged on a basic column (Waters, Oasis MAX, 500 mg) and washed with 1; 0.1M NaOH. 2; 50% CH₃CN/H₂O. 3; 100% CH₃CN and eluted and collected with 90% CH₃CN/2% formic acid. After freeze drying this yielded a white solid of ethyl 5-cyano-2-(difluoromethyl)-6-[3-({[4-(hydroxymethyl)benzyl]sulfonyl}carbamoyl) azetidin-1-yl]nicotinate. Yield: 59 mg (70%).

¹H NMR (400 MHz, DMSO-d₆) δ 1.31 (3H, t, J=7.1 Hz), 3.51-3.67 (1H, m), 4.28 (2H, q, J=7.1 Hz), 4.34-4.43 (2H, m), 4.43-4.55 (4H, m), 4.72 (2H, s), 5.15-5.25 (1H, m), 7.25-7.57 (1H, m), 7.29 (2H, d, J=8.3 Hz), 7.32 (2H, d, J=8.3 Hz), 8.48 (1H, s), 11.74-11.88 (1H, br s). MS m/z: 509 (M+1).

### Example 131

### Ethyl 5-cyano-2-(difluoromethyl)-6-[4-({[4-(hydroxymethyl)benzyl]sulfonyl}carbamoyl)piperidin-1-yl]nicotinate

Prepared essentially to example 130 using 1-[3-cyano-6-(difluoromethyl)-5-(ethoxycarbonyl)pyridin-2-yl]piperidine-4-carboxylic acid in step (d) followed by step (e) to give ethyl 5-cyano-2-(difluoromethyl)-6-[4-({[4-(hydroxymethyl)benzyl]sulfonyl}carbamoyl)piperidin-1-yl]nicotinate as a white solid. Yield: 66 mg (76%).

¹H NMR (400 MHz, DMSO-d₆) δ 1.32 (3H, t, J=7.1 Hz), 1.60-1.75 (2H, m), 1.82-1.94 (2H, m), 2.54-2.72 (1H, m), 3.12-3.31 (2H, m), 4.29 (2H, q, J=7.1 Hz), 4.50 (2H, d, J=5.4 Hz), 4.54-4.63 (2H, m), 4.67 (2H, s), 5.22 (1H, t, J=5.7 Hz), 7.24 (2H, d, J=8.1 Hz), 7.34 (2H, d, J=8.1 Hz), 7.41 (1H, t, J=54.0 Hz), 8.51 (1H, s), 11.53-11.70 (1H, br s). MS m/z: 537 (M+1).

### Example 132

### Ethyl 6-{3-[(benzylsulfonyl)carbamoyl]azetidin-1-yl}-5-cyano-2-(2,2-difluoroethoxy)nicotinate

### (a) 1-(Trifluoroacetyl)azetidine-3-carboxylic acid

Trifluoroacetic anhydride (93.5 g, 445 mmol) was added to solid acetidine-3-carboxylic acid (15 g, 148 mmol) at 0° C. (ice/water bath cooling). The mixture was stirred manually with a spatula for 30 minutes followed by mechanical stirring (the mixture became homogenous after 40 minutes) for another 2 hours and 40 minutes. The mixture was concentrated in vacuo and the residual yellow oil was partitioned between EtOAc (300 mL) and water (50 mL). The phases was separated and the organic phase was washed with water (2×50 mL) and Brine (20 mL), dried (Na₂SO₄), filtered and evaporated to give a yellow oil. Drying in vacuo at r.t over night gave the product as a yellow solid. Yield: 29.2 g (100%).

### (b) tert-Butyl 1-(trifluoroacetyl)azetidine-3-carboxylate

1,1-di-tert-butoxy-N,N-dimethylmethanamine (16.5 g, 81 mmol) was added to a solution of 1-(trifluoroacetyl)azetidine-3-carboxylic acid (5 g, 25 mmol) and the mixture was heated to reflux for 8 hours. LC-MS showed remaining starting material and therefore an additional amount of 1,1-di-tert-butoxy-N,N-dimethylmethanamine (21.2 g, 81 mmol) was added and the heating was continued over night. LC-MS showed still some remaining starting material (starting material/product about ½) and the THF was exchanged for toluene (100 mL) and the mixture heated to 100° C. (oil bath temperature) for 2 hours. The solvent was evaporated and the residue dissolved in EtOAc (200 mL). The organic phase was washed with NaHCO₃(sat) (2×50 mL), water (2×50 mL), Brine (50 mL), dried (Na₂SO₄), filtered and evaporated to give the desired product. Yield: 4.5 g (70%).

### (c) tert-Butyl azetidine-3-carboxylate

Potassium carbonate (7.37 g, 53.3 mmol) was added to a solution of tert-butyl 1-(trifluoroacetyl)azetidine-3-carboxylate (4.5 g, 17.8 mmol) in methanol/water (7/3, 71 mL) and the mixture was stirred at r.t for 3.5 hours. The methanol was evaporated and DCM (200 mL) was added. The phases were separated and the water phase was extracted with DCM (2×100 mL). The combined organic phase was washed with water (2×50 mL), brine (1×50 mL), dried (Na₂SO₄), filtered and evaporated to give the desired product as a yellow oil. Yield: 1.19 g (40%).

### (d) tert-Butyl 1-(2-cyanoethanimidoyl)azetidine-3-carboxylate

A microwave vial was charged with tert-butyl azetidine-3-carboxylate (1.1 g, 6.65 mmol, 95% pure), ethyl 2-cyanoethanimidoate (See McElvain, S. M.; Schroeder, J. P.; J. Am. Chem. Soc. 71, p. 40 (1949)) (1.12 g, 7.98 mmol, 80% pure) and EtOH (15 mL) and heated to 100° C. for 10 minutes. This mixture was used as such in the next step assuming 100% yield.

### (e) Ethyl 6-[3-(tert-butoxycarbonyl)azetidin-1-yl]-5-cyano-2-oxo-1,2-dihydropyridine-3-carboxylate

Diethyl (ethoxymethylene)malonate (2.16 g, 9.98 mmol) was added to the solution from step (d) above and the reaction mixture was stirred at r.t for 18 hours followed by 10 minutes at 100° C. and 10 minutes at 110° C. using microwave single node heating. The solvent was evaporated and the residue was dissolved in DCM and passed through a plug of silica gel (Eluted with DCM (100%), DCM/MeOH (10/1), (5/1) and (1/1). The fractions containing the product was collected and evaporated to give a crude product (3.1 g). The crude product was purified by preparative HPLC (Kromasil C₈, 10 μm, using a gradient of 25 to 70% CH₃CN/0.2% AcOH in water) to give ethyl 6-[3-(tert-butoxycarbonyl)azetidin-1-yl]-5-cyano-2-oxo-1,2-dihydropyridine-3-carboxylate as a solid. Yield: 1.043 g (36%).

### (f) Ethyl 6-[3-(tert-butoxycarbonyl)azetidin-1-yl]-5-cyano-2-(2,2-difluoroethoxy)nicotinate

Ethyl 6-[3-(tert-butoxycarbonyl)azetidin-1-yl]-5-cyano-2-oxo-1,2-dihydropyridine-3-carboxylate (200 mg, 0.576 mmol) and Ag₂CO₃ (397 mg, 1.44 mmol) were dissolved in DMSO (15 mL) and after 5 min in rt, 2-Iodo-1,1-Difluoroethane (2.21 g, 11.5 mmol) was added. The reaction mixture was heated to 95° C. over night. LCMS shows product and no SM left. The mixture was filtered and diluted with water and extracted with DCM (×3) and EtOAc (×1). The combined organics was run through a phase separator and concentrated under reduced pressure to give the desired product. The crude product was used without further purification. Assumed quantitative yield. MS m/z: 412 (M+1).

### (g) 1-[3-Cyano-6-(2,2-difluoroethoxy)-5-(ethoxycarbonyl)pyridin-2-yl]azetidine-3-carboxylic acid

Ethyl 6-[3-(tert-butoxycarbonyl)azetidin-1-yl]-5-cyano-2-(2,2-difluoroethoxy)nicotinate (237 mg, 0.576 mmol) was dissolved in 90% Formic acid (9 mL) and the reaction mixture was stirred at rt over night. Concentrated and co-concenrated from DCM and freeze dried to give 1-[3-cyano-6-(2,2-difluoroethoxy)-5-(ethoxycarbonyl)pyridin-2-yl]azetidine-3-carboxylic acid as a solid. Yield: 194 mg (95%).

¹H NMR (500 MHz, DMSO-d₆): δ 1.25 (3H, t, J=7.1 Hz), 3.53-3.61 (1H, m), 4.17 (2H, q, J=7.1 Hz), 4.32-4.42 (2H, m), 4.46-4.56 (2H, m), 4.60 (2H, td, J=14.8, 3.5 Hz), 6.37 (1H, tt, J=54.6, 3.5 Hz), 8.27 (1H, s), 12.83 (1H, s). MS m/z: 356 (M+1).

### (h) Ethyl 6-{3-[(benzylsulfonyl)carbamoyl]azetidin-1-yl}-5-cyano-2-(2,2-difluoroethoxy)nicotinate

1-phenylmethanesulfonamide (18.8 mg, 0.11 mmol) was charged in a 16 mL vial and PyBrop (70 mg, 0.15 mmol) dissolved in DCM (1 mL) was added. 1-[3-cyano-6-(2,2-difluoroethoxy)-5-(ethoxycarbonyl)pyridin-2-yl]azetidine-3-carboxylic acid (35.5 mg, 0.11 mmol) dissolved in DCM (2 mL) and DIPEA (0.17 mL, 1.0 mmol) was added. The reaction mixture was stirred at rt for 40 min. The mixture was washed with 1% KHSO₄ solution (1 mL) and the aqueous phase was extracted with DCM (0.5 mL). The combined organics was passed through a phase separator and evaporated in vacuum centrifuge. The crude was purified by preparative HPLC (Waters Fraction Lynx II Purification System. Column: Sunfire Prep C₁₈, 5 μm OBD, 19×150 mm columns. Gradient: 5-95% MeCN in 0.11 mM HCOOH, pH3. MS triggered fraction collection was used. Mass spectra were recorded on either a Micromass ZQ single quadrupole or a Micromass Quattro micro, both equipped with a pneumatically assisted electrospray interface.) to give ethyl 6-{3-[(benzylsulfonyl)carbamoyl]azetidin-1-yl}-5-cyano-2-(2,2-difluoroethoxy)nicotinate. Yield: 28.3 mg (50%).

¹H NMR (600 MHz, DMSO-d₆): δ 1.24 (3H, t, J=7.1 Hz), 3.50-3.56 (1H, m), 4.16 (2H, q, J=7.1 Hz), 4.23-4.43 (4H, m), 4.56-4.63 (2H, m), 4.72 (2H, s), 6.37 (1H, t, J=55.5 Hz), 7.29-7.36 (5H, m), 8.28 (1H, s). MS m/z: 509 (M+1).

### Example 133

### ethyl 5-cyano-2-(2,2-difluoroethoxy)-6-(3-{[(4-fluorobenzyl)sulfonyl]carbamoyl}azetidin-1-yl)nicotinate

Prepared according to the procedure in example 132 (h) using 1-(4-fluorophenyl)methanesulfonamide to give ethyl 5-cyano-2-(2,2-difluoroethoxy)-6-(3-{[(4-fluorobenzyl)sulfonyl]carbamoyl}azetidin-1-yl)nicotinate. Yield: 32 mg (55%).

¹H NMR (600 MHz, DMSO-d₆): δ 1.24 (3H, t, J=7.1 Hz), 3.51-3.57 (1H, m), 4.16 (2H, q, J=7.1 Hz), 4.24-4.33 (2H, m), 4.35-4.46 (2H, m), 4.56-4.63 (2H, m), 4.73 (2H, s), 6.37 (1H, t, J=55.0 Hz), 7.17-7.21 (2H, m), 7.35-7.40 (2H, m), 8.27 (1H, s). MS m/z: 527 (M+1).

### Example 134

### Ethyl 5-cyano-2-(2,2-difluoroethoxy)-6-(3-{[(2-fluorobenzyl)sulfonyl]carbamoyl}azetidin-1-yl)nicotinate

Prepared according to the procedure in Example 132 (h) using 1-(2-fluorophenyl)methanesulfonamide to give ethyl 5-cyano-2-(2,2-difluoroethoxy)-6-(3-{[(2-fluorobenzyl)sulfonyl]carbamoyl}azetidin-1-yl)nicotinate. Yield: 33.2 mg (57%).

¹H NMR (600 MHz, DMSO-d₆): δ 1.24 (3H, t, J=7.3 Hz), 3.54-3.60 (1H, m), 4.16 (2H, q, J=7.3 Hz), 4.29-4.50 (4H, m), 4.57-4.64 (2H, m), 4.78 (2H, s), 6.37 (1H, t, J=54.2 Hz), 7.20-7.25 (2H, m), 7.41-7.46 (2H, m), 8.28 (1H, s). MS m/z: 527 (M+1).

### Example 135

### Ethyl 5-cyano-6-(3-{[(2,4-difluorobenzyl)sulfonyl]carbamoyl}azetidin-1-yl)-2-(2,2-difluoroethoxy)nicotinate

Prepared according to the procedure in Example 132 (h) using 1-(2,4-difluorophenyl)methanesulfonamide to give ethyl 5-cyano-2-(2,2-difluoroethoxy)-6-(3-{[(2,4-difluorobenzyl)sulfonyl]carbamoyl}azetidin-1-yl)nicotinate. Yield: 33.4 mg (55%)

¹H NMR (600 MHz, DMSO-d₆): δ 1.23 (3H, t, J=7.0 Hz), 3.52-3.59 (1H, m), 4.16 (2H, q, J=7.0 Hz), 4.22-4.32 (2H, m), 4.36-4.47 (2H, m), 4.55-4.62 (2H, m), 4.76 (2H, s), 6.36 (1H, t, J=54.2 Hz), 7.18-7.21 (1H, m), 7.39-7.46 (2H, m), 8.27 (1H, s). MS m/z: 545 (M+1).

### Example 136

### Isopropyl 6-{3-[(benzylsulfonyl)carbamoyl]azetidin-1-yl}-5-cyano-2-(difluoromethyl)nicotinate

### (a) 6-{3-[(Benzylsulfonyl)carbamoyl]azetidin-1-yl}-5-cyano-2-(difluoromethyl)nicotinic acid

Ethyl 6-(3-{[(benzylsulfonyl)amino]carbonyl}azetidin-1-yl)-5-cyano-2-(difluoromethyl)nicotinate (15.5 mg, 0.032 mmol) was suspensioned in 1M NaOH (0.4 mL, 0.4 mmol) and CH₃CN (0.1 mL) was added. The reaction mixture was stirred at rt for 1 h. The mixture was diluted with water, made acidic with formic acid and extracted with EtOAc (×3). The combined organics was evaporated and the crude product was used without further purification. Assumed quantitative yield. MS m/z: 451 (M+1).

### (b) Isopropyl 6-{3-[(benzylsulfonyl)carbamoyl]azetidin-1-yl}-5-cyano-2-(difluoromethyl)nicotinate

6-{3-[(Benzylsulfonyl)carbamoyl]azetidin-1-yl}-5-cyano-2-(difluoromethyl)nicotinic acid (14.6 mg, 0.032 mmol), DMAP (4.4 mg, 0.036 mmol) and EDC (6.8 mg, 0.036 mmol) were suspended in IPA (2 mL) and TEA (5 μL, 0.032 mmol) was added. The reaction mixture was stirred at 50° C. over night. HATU (12.2 mg, 0.032 mmol) was added at 50° C. and the reaction mixture was stirred at 50° C. for 4 h. The mixture was diluted with DCM, washed with 1% KHSO₄ solution and the aqueous phase was extracted with DCM (×3). The combined organics was concentrated under reduced pressure and the crude product was purified by preparative HPLC (Kromasil C₈ 10 μm, 21.5×250 mm column, eluent A: 100% acetonitrile, eluent B: 0.2% acetic acid in water containing 5% acetonitrile, flow 25 mL/min, using a gradient of 30-100% eluent A over 30 minutes) to give isopropyl 6-{3-[(benzylsulfonyl)carbamoyl]azetidin-1-yl}-5-cyano-2-(difluoromethyl)nicotinate as a white solid. Yield: 3 mg, (19%).

¹H NMR (400 MHz, DMSO-d₆): δ 1.30 (6H, d, J=6.3 Hz), 4.34 (2H, br s), 4.37-4.49 (2H, m), 4.51-4.67 (2H, m), 5.08 (1H, quintet, J=6.3 Hz), 7.31 (5H, br s), 7.38 (1H, t, J=54.3 Hz), 8.43 (1H, s). Note! One H signal is overlapping with the DMSO signal. MS m/z: 493 (M+1), 491 (M−1).

### Example 137

### Ethyl 5-cyano-6-[3-({[(4-methylcyclohexyl)methyl]sulfonyl}carbamoyl)azetidin-1-yl]-2-(trifluoromethyl)nicotinate

Prepared according to Method D from 1-[3-cyano-5-(ethoxycarbonyl)-6-(trifluoromethyl)pyridin-2-yl]azetidine-3-carboxylic acid and 1-(4-methylcyclohexyl)methanesulfonamide to give ethyl 5-cyano-6-[3-({[(4-methylcyclohexyl)methyl]sulfonyl}carbamoyl)azetidin-1-yl]-2-(trifluoromethyl)nicotinate. Yield: 43 mg (55%). MS m/z: 517 (M+1).

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, and the like) cited in the present application is incorporated herein by reference in its entirety.

What is claimed is:

1. A compound of formula I or a pharmaceutically acceptable salt thereof: 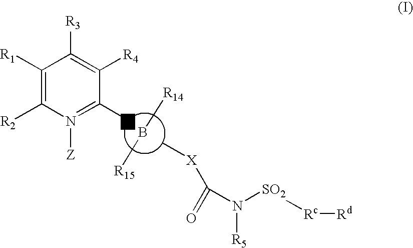
wherein:
R₁ represents R₆OC(O), R₇C(O), R₁₆SC(O), R₁₇S, R₁₈C(S) or a group gII 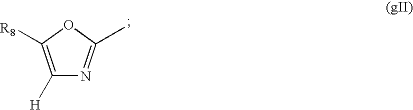
R₂ represents (C₁-C₁₂)alkyl optionally interrupted by oxygen and wherein the alkyl is substituted by one or more halogen atoms; or
R₂ represents (C₁-C₁₂)alkoxy substituted by one or more halogen atoms;
R₃ represents H, CN, NO₂, halogen, (C₁-C₁₂)alkyl optionally interrupted by oxygen and/or optionally substituted by OH, aryl, cycloalkyl, heterocyclyl or one or more halogen atoms; or
R₃ represents (C₁-C₁₂)alkoxy optionally substituted by one or more halogen atoms; or
R₃ represents (C₃-C₆)cycloalkyl, hydroxy(C₁-C₁₂)alkyl, (C₁-C₁₂)alkylC(O), (C₁-C₁₂)alkylthiOC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(3)}R^{b(3)} in which R^{a(3)} and R^{b(3)} independently represent H, (C₁-C₁₂)alkyl, (C₁-C₁₂)alkylC(O) or R^{a(3)} and R^{b(3)} together with the nitrogen atom represent piperidine, pyrrolidine, azetidine or aziridine;
R₄ represents H, CN, NO₂, halogen, (C₁-C₁₂)alkyl optionally interrupted by oxygen and/or optionally substituted by OH, COOH, (C₁-C₆)alkoxycarbonyl, aryl, cycloalkyl, heterocyclyl or one or more halogen atoms; or
R₄ represents (C₃-C₆)cycloalkyl, hydroxy(C₁-C₁₂)alkyl, (C₁-C₁₂)alkylC(O), (C₁-C₁₂)alkylcycloalkyl, (C₁-C₁₂)alkoxy wherein the alkoxy group may optionally be substituted by one or more halogen atoms, OH and/or COOH and/or (C₁-C₆)alkoxycarbonyl; or
R₄ represents (C₁-C₁₂)alkylthiOC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(4)}R^{b(4)} in which R^{a(4)} and R^{b(4)} independently represent H, (C₁-C₁₂)alkyl, (C₁-C₁₂)alkylC(O) or R^{a(4)} and R^{b(4)} together with the nitrogen atom represent piperidine, pyrrolidine, azetidine or aziridine;
Z represents O or is absent;
R₅ represents H or (C₁-C₁₂)alkyl;
R₆ represents (C₁-C₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; or
R₆ represents (C₃-C₆)cycloalkyl, hydroxy(C₂-C₁₂)alkyl, aryl or heterocyclyl;
R₇ represents (C₁-C₁₂)alkyl optionally interrupted by oxygen, and/or optionally substituted by OH, aryl, cycloalkyl, heterocyclyl or one or more halogen atoms; or
R₇ represents (C₃-C₆)cycloalkyl, hydroxy(C₁-C₁₂)alkyl, aryl or heterocyclyl;
R₈ represents H, (C₁-C₁₂)alkyl optionally interrupted by oxygen, and/or optionally substituted by aryl, cycloalkyl, heterocyclyl or one or more halogen atoms; or
R₈ represents (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;
R₁₄ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen atoms, OH, aryl, cycloalkyl and heterocyclyl; or
R₁₄ represents aryl, heterocyclyl, one or more halogen atoms, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl, a group of formula NR^{a(14)}R^{b(14)} in which R^{a(14)} and R^{b(14)} independently represent H, (C₁-C₁₂)alkyl, (C₁-C₁₂)alkylC(O), (C₁-C₁₂)alkoxyC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen atoms, OH, aryl, cycloalkyl and heterocyclyl; or
R₁₅ represents aryl, heterocyclyl, one or more halogen atoms, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(15)}R^{b(15)} in which R^{a(15)} and R^{b(15)} independently represent H, (C₁-C₁₂)alkyl, (C₁-C₁₂)alkylC(O)), (C₁-C₁₂)alkoxyC(O) or R^{a(15)} and R^{b(15)} together with the nitrogen atom represent piperidine, pyrrolidine, azetidine or aziridine;
R₁₆ represents (C₁-C₁₂)alkyl optionally interrupted by oxygen and/or optionally substituted by OH, aryl, cycloalkyl, heterocyclyl or one or more halogen atoms; or
R₁₆ represents (C₃-C₆)cycloalkyl, hydroxy(C₂-C₁₂)alkyl, (C₁-C₁₂)alkoxy, (C₃-C₆)cycloalkoxy, aryl or heterocyclyl;
R₁₇ represents (C₁-C₁₂)alkyl optionally interrupted by oxygen and/or optionally substituted by OH, aryl, cycloalkyl, heterocyclyl or one or more halogen atoms; or
R₁₇ represents (C₃-C₆)cycloalkyl, hydroxy(C₁-C₁₂)alkyl, (C₁-C₁₂)alkoxy, (C₃-C₆)cycloalkoxy, aryl or heterocyclyl;
R₁₈ represents (C₁-C₁₂)alkyl optionally interrupted by oxygen and/or optionally substituted by OH, aryl, cycloalkyl, heterocyclyl or one or more halogen atoms; or
R₁₈ represents (C₃-C₆)cycloalkyl, hydroxy(C₁-C₁₂)alkyl, (C₁-C₁₂)alkoxy, (C₃-C₆)cycloalkoxy, aryl or heterocyclyl;
R^{c} is absent or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogen (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; or
R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above;
R₁₉, when present, represents H or (C₁-C₄)alkyl;
R^{d} represents (C₁-C₁₂)alkyl, (C₃-C₈)cycloalkyl, aryl or heterocyclyl, and anyone of these groups optionally substituted with one or more halogen atoms and/or one or more of the following groups: OH, CN, NO₂, (C₁-C₁₂)alkyl, (C₁-C₁₂)alkoxyC(O), (C₁-C₁₂)alkoxy, halogen substituted (C₁-C₁₂)alkyl, halogen substituted (C₁-C₁₂)alkoxy, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;
X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆)alkyl; or
X represents a group (—CH₂—)ₙ wherein n=2-6, which optionally is unsaturated and/or substituted by one or more substituent chosen among halogen, hydroxyl or (C₁-C₆)alkyl; and
B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) with the proviso that B is not piperazine, and further the B-ring/ring system is connected to X in another of its positions; the substituents R₁₄ and R₁₅ are connected to the B ring/ring system in such a way that no quarternary ammonium compounds are formed (by these connections).

2. A compound according to claim 1 wherein:
R₁ represents R₆OC(O), R₇C(O), R₁₆SC(O), R₁₇S, R₁₈C(S) or a group gII, 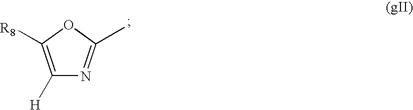
R₂ represents (C₁-C₆)alkyl optionally interrupted by oxygen and wherein the alkyl is substituted by one or more halogen atoms; or
R₂ represents (C₁-C₆)alkoxy substituted by one or more halogen atoms;
R₃ represents H, CN, NO₂, halogen, (C₁-C₆)alkyl optionally interrupted by oxygen and/or optionally substituted by OH, aryl, cycloalkyl, heterocyclyl or one or more halogen atoms; or
R₃ represents (C₁-C₆)alkoxy optionally substituted by one or more halogen atoms; or
R₃ represents (C₃-C₆)cycloalkyl, hydroxy(C₁-C₆)alkyl, (C₁-C₆)alkylC(O), (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents H, CN, NO₂, halogen, (C₁-C₆)alkyl optionally interrupted by oxygen and/or optionally substituted by OH, COOH, (C₁-C₆)alkoxycarbonyl, aryl, cycloalkyl, heterocyclyl or one or more halogen atoms; or
R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by one or more halogen atoms, OH and/or COOH and/or (C₁-C₃)alkoxycarbonyl; or
R₄ represents (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;
R₅ represents H or (C₁-C₆)alkyl;
R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen atoms; or
R₆ represents (C₃-C₆)cycloalkyl, hydroxy(C₂-C₆)alkyl, aryl or heterocyclyl;
R₇ represents (C₁-C₆)alkyl optionally interrupted by oxygen, and/or optionally substituted by OH, aryl, cycloalkyl, heterocyclyl or one or more halogen atoms; or
R₇ represents (C₃-C₆)cycloalkyl, hydroxy(C₁-C₆)alkyl, aryl or heterocyclyl;
R₈ represents H, (C₁-C₆)alkyl optionally interrupted by oxygen, and/or optionally substituted by aryl, cycloalkyl, heterocyclyl or one or more halogen atoms; or
R₈ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₄ represents aryl, heterocyclyl, one or more halogen atoms, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₅ represents aryl, heterocyclyl, one or more halogen atoms, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;
R₁₆ represents (C₁-C₆)alkyl optionally interrupted by oxygen and/or optionally substituted by OH, aryl, cycloalkyl, heterocyclyl or one or more halogen atoms; or
R₁₆ represents (C₃-C₆)cycloalkyl, hydroxy(C₂-C₆)alkyl, (C₁-C₆)alkoxy, (C₃-C₆)cycloalkoxy, aryl, or heterocyclyl;
R₁₇ represents (C₁-C₆)alkyl optionally interrupted by oxygen and/or optionally substituted by OH, aryl, cycloalkyl, heterocyclyl or one or more halogen atoms; or
R₁₇ represents (C₃-C₆)cycloalkyl, hydroxy(C₁-C₆)alkyl, (C₁-C₆)alkoxy, (C₃-C₆)cycloalkoxy, aryl or heterocyclyl;
R₁₈ represents (C₁-C₆)alkyl optionally interrupted by oxygen and/or optionally substituted by OH, aryl, cycloalkyl, heterocyclyl or one or more halogen atoms; or
R₁₈ represents (C₃-C₆)cycloalkyl, hydroxy(C₁-C₆)alkyl, (C₁-C₆)alkoxy, (C₃-C₆)cycloalkoxy, aryl or heterocyclyl;
R₁₉, when present, represents H or (C₁-C₄)alkyl; and
R^{d} represents (C₁-C₆)alkyl, (C₃-C₈)cycloalkyl, aryl or heterocyclyl, and anyone of these groups optionally substituted with one or more halogen atoms and/or one or more of the following groups: OH, CN, NO₂, (C₁-C₆)alkyl, (C₁-C₆)alkoxyC(O), (C₁-C₆)alkoxy, halogen substituted (C₁-C₆)alkyl, halogen substituted (C₁-C₆)alkoxy, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine.

3. A compound according to claim 2 wherein:
R₁ represents R₆OC(O), R₁₆SC(O), or a group gII, 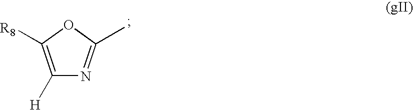
R₃ represents H, CN, NO₂, halogen, (C₁-C₆)alkyl optionally interrupted by oxygen and/or optionally substituted by OH, aryl, cycloalkyl, heterocyclyl or one or more halogen atoms; or
R₃ represents (C₁-C₆)alkoxy optionally substituted by one or more halogen atoms; or
R₃ represents (C₃-C₆)cycloalkyl, hydroxy(C₁-C₆)alkyl, (C₁-C₆)alkylC(O), (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents H, CN, NO₂, halogen, (C₁-C₆)alkyl optionally interrupted by oxygen and/or optionally substituted by OH, COOH, aryl, cycloalkyl, heterocyclyl or one or more halogen atoms; or
R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by one or more halogen atoms, OH and/or COOH and/or methoxycarbonyl; or
R₄ represents (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;
R₈ represents H, (C₁-C₆)alkyl optionally interrupted by oxygen, and/or optionally substituted by aryl, cycloalkyl, heterocyclyl or one or more halogen atoms; or
R₈ represents (C₃-C₆)cycloalkyl, hydroxy(C₁-C₆)alkyl, (C₁-C₆)alkoxy, (C₃-C₆)cycloalkoxy, aryl or heterocyclyl;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₄ represents aryl, heterocyclyl, one or more halogen atoms, (C₃-C₆)cycloalkyl, hydroxy(C₁-C₆)alkyl, (C₁-C₆)alkoxy, (C₃-C₆)cycloalkoxy, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₅ represents aryl, heterocyclyl, one or more halogen atoms, (C₃-C₆)cycloalkyl, hydroxy(C₁-C₆)alkyl, (C₁-C₆)alkoxy, (C₃-C₆)cycloalkoxy, or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;
R₁₆ is ethyl; and
R^{d} represents (C₁-C₆)alkyl, (C₃-C₈)cycloalkyl, aryl or heterocyclyl, and anyone of these groups optionally substituted with one or more halogen atoms and/or one or more of the following groups: CN, NO₂, (C₁-C₆)alkyl, (C₁-C₆)alkoxy, halogen substituted (C₁-C₆)alkyl, halogen substituted (C₁-C₆)alkoxy, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl.

4. A compound according to claim 1 wherein:
R₁ represents R₆OC(O);
R₂ represents (C₁-C₄)alkyl substituted by one or more halogen atoms;
R₃ represents H;
R₄ represents CN or halogen;
Z is absent;
R₅ represents H;
R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms;
R₁₄ represents H;
R₁₅ represents H;
R^{c} is absent or represents an unsubstituted (C₁-C₄)alkylene group;
R^{d} represents (C₁-C₆)alkyl, (C₃-C₈)cycloalkyl, aryl or heterocyclyl, and any one of these groups optionally substituted with one or more halogen atoms and/or one or more of the following groups: CN, (C₁-C₆)alkyl, (C₁-C₆)alkoxy, halogen substituted (C₁-C₆)alkyl, halogen substituted (C₁-C₆)alkoxy;
X represents a single bond or methylene (—CH₂—); and
B is a monocyclic, 4 to 7-membered heterocyclic ring/ring system comprising one or more nitrogen and optionally one or more atoms selected from oxygen or sulphur, which nitrogen is connected to the pyridine-ring (according to formula I) with the proviso that B is not piperazine, and further the B-ring/ring system is connected to X in another of its positions; the substituents R₁₄ and R₁₅ are connected to the B ring/ring system in such a way that no quarternary ammonium compounds are formed (by these connections).

5. A compound according to claim 1 wherein:
R₁ is ethoxycarbonyl or isopropoxycarbonyl;
R₂ is chosen from a group consisting of fluoromethyl, chloromethyl, difluoromethyl, trifluoromethyl, pentafluoroethyl, 1-fluoroethyl, 2-fluoroethoxy, 2,2,2,-trifluoroethoxy, difluoromethoxy and 2,2-difluoroethoxy;
R₃ is H;
R₄ is chosen from chloro or cyano;
Z is absent;
R₅ is H;
R₆ is ethyl or isopropyl;
R₁₄ is H;
R₁₅ is H;
R^{c} is absent or is chosen from methylene (—CH₂—) or ethylene (—CH₂CH₂—);
R^{d} is chosen from a group consisting of n-butyl, 4-methylcyclohexyl, phenyl, 3-methylphenyl, 4-methylphenyl, 2-(trifluoromethoxy)phenyl, 4-(trifluoromethoxy)phenyl, 2-fluorophenyl, 3-fluorophenyl, 4-fluorophenyl, 2-chlorophenyl, 3-chlorophenyl, 4-chlorophenyl, 2,4-dichlorophenyl, 2-cyanophenyl, 3-cyanophenyl, 4-cyanophenyl, 3-methoxyphenyl, 2-naphtyl, 2,6-difluorophenyl, 4-fluoro-3-methylphenyl, 2-chloro-4-fluorophenyl, 2,3,6-trifluorophenyl, 2,4-difluorophenyl, 4-chloro-2-fluorophenyl, 5-fluoro-2-methylphenyl, 2-fluoro-5-methylphenyl, 3-methoxyphenyl, 3,4-difluorophenyl, 4-hydroxymethylphenyl and 5-chloro-2-thienyl;
X represents a single bond or methylene (—CH₂—); and
B is chosen from the group consisting of 4-piperidin-1-ylene, 3-pyrrolidine-1-ylene and 3-azetidin-1-ylene, and the substituents R₁₄ and R₁₅ are connected to the B ring/ring system, in such a way that no quarternary ammonium compounds are formed (by these connections).

6. A compound according to claim 1 which is of the formula (Ia):

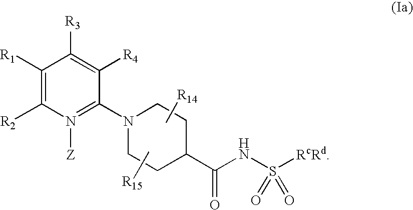

7. A compound according to claim 1 which is of the formula (Ib):

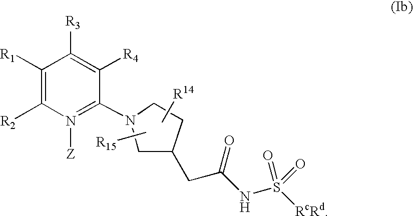

8. A compound according to claim 1 which is of the formula (Ic):

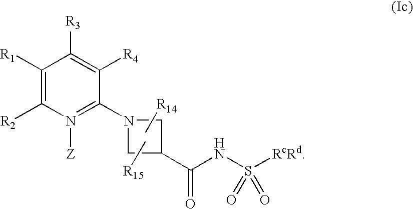

9. A compound according to claim 1 which is of the formula (Id):

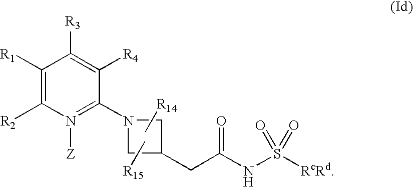

10. A compound according to claim 1 wherein Z is absent.

11. A compound according to claim 1 wherein Z is O.

12. A compound according to claim 1 wherein R₁ represents R₆OC(O).

13. A compound according to claim 12 which is of the formula (Iaa):

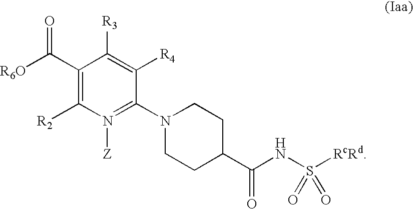

14. A compound according to claim 12 which is of the formula (Ibb):

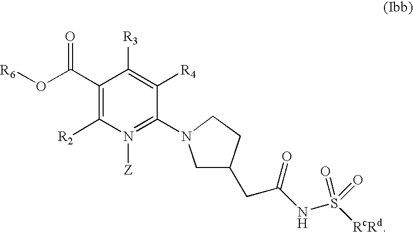

15. A compound according to claim 12 which is of the formula (Icc):

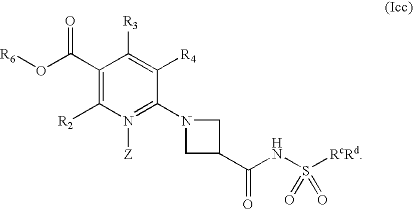

16. A compound according to claim 12 which is of the formula (Idd):

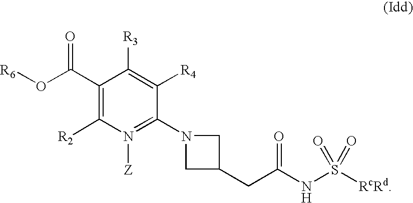

17. A compound according to claim 1 wherein R₁ represents R₆OC(O), R₁₆SC(O) or a group gII

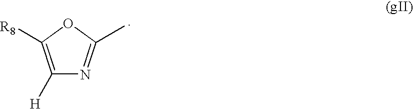

18. A compound selected from:
ethyl 6-(4-{[(benzylsulfonyl)amino]carbonyl}piperidin-1-yl)-5-chloro-2-(difluoromethyl)nicotinate;
ethyl 6-(4-{[(benzylsulfonyl)amino]carbonyl}piperidin-1-yl)-5-cyano-2-(difluoromethyl)nicotinate;
ethyl 6-(4-{[(benzylsulfonyl)amino]carbonyl}piperidin-1-yl)-5-cyano-2-(trifluoromethyl)nicotinate;
ethyl 6-(3-{[(benzylsulfonyl)amino]carbonyl}azetidin-1-yl)-5-cyano-2-(difluoromethyl)nicotinate;
ethyl 6-(3-{[(benzylsulfonyl)amino]carbonyl}azetidin-1-yl)-5-cyano-2-(trifluoromethyl)nicotinate;
ethyl 6-(4-{[(benzylsulfonyl)amino]carbonyl}piperidin-1-yl)-5-cyano-2-(fluoromethyl)nicotinate;
ethyl 6-(3-{[(benzylsulfonyl)amino]carbonyl}azetidin-1-yl)-5-cyano-2-(fluoromethyl)nicotinate;
ethyl 5-cyano-2-(difluoromethyl)-6-{4-[({[(4-methylcyclohexyl)methyl]sulfonyl}amino)carbonyl]piperidin-1-yl}nicotinate;
ethyl 5-cyano-2-(difluoromethyl)-6-[3-({[(2-fluorobenzyl)sulfonyl]amino}carbonyl)azetidin-1-yl]nicotinate;
ethyl 5-cyano-2-(difluoromethyl)-6-[4-({[(2-fluorobenzyl)sulfonyl]amino}carbonyl)piperidin-1-yl]nicotinate;
ethyl 5-cyano-2-(difluoromethyl)-6-[4-({[(3-fluorobenzyl)sulfonyl]amino}carbonyl)piperidin-1-yl]nicotinate;
ethyl 5-cyano-2-(difluoromethyl)-6-[4-({[(4-fluorobenzyl)sulfonyl]amino}carbonyl)piperidin-1-yl]nicotinate;
ethyl 6-[4-({[(2-chlorobenzyl)sulfonyl]amino}carbonyl)piperidin-1-yl]-5-cyano-2-(difluoromethyl)nicotinate;
ethyl 6-[4-({[(3-chlorobenzyl)sulfonyl]amino}carbonyl)piperidin-1-yl]-5-cyano-2-(difluoromethyl)nicotinate;
ethyl 6-[4-({[(4-chlorobenzyl)sulfonyl]amino}carbonyl)piperidin-1-yl]-5-cyano-2-(difluoromethyl)nicotinate;
ethyl 5-cyano-2-(difluoromethyl)-6-[4-({[(3-methylbenzyl)sulfonyl]amino}carbonyl)piperidin-1-yl]nicotinate;
ethyl 5-cyano-2-(difluoromethyl)-6-[4-({[(4-methylbenzyl)sulfonyl]amino}carbonyl)piperidin-1-yl]nicotinate;
ethyl 5-cyano-6-[4-({[(2,4-dichlorobenzyl)sulfonyl]amino}carbonyl)piperidin-1-yl]-2-(difluoromethyl)nicotinate;
ethyl 5-cyano-2-(difluoromethyl)-6-[3-({[(3-fluorobenzyl)sulfonyl]amino}carbonyl)azetidin-1-yl]nicotinate;
ethyl 5-cyano-2-(difluoromethyl)-6-[3-({[(4-fluorobenzyl)sulfonyl]amino}carbonyl)azetidin-1-yl]nicotinate;
ethyl 6-[3-({[(2-chlorobenzyl)sulfonyl]amino}carbonyl)azetidin-1-yl]-5-cyano-2-(difluoromethyl)nicotinate;
ethyl 6-[3-({[(3-chlorobenzyl)sulfonyl]amino}carbonyl)azetidin-1-yl]-5-cyano-2-(difluoromethyl)nicotinate;
ethyl 6-[3-({[(4-chlorobenzyl)sulfonyl]amino}carbonyl)azetidin-1-yl]-5-cyano-2-(difluoromethyl)nicotinate;
ethyl 5-cyano-2-(difluoromethyl)-6-[3-({[(3-methylbenzyl)sulfonyl]amino}carbonyl)azetidin-1-yl]nicotinate;
ethyl 5-cyano-2-(difluoromethyl)-6-[3-({[(4-methylbenzyl)sulfonyl]amino}carbonyl)azetidin-1-yl]nicotinate;
ethyl 5-cyano-6-[3-({[(2,4-dichlorobenzyl)sulfonyl]amino}carbonyl)azetidin-1-yl]-2-(difluoromethyl)nicotinate;
ethyl 5-cyano-2-(difluoromethyl)-6-{3-[({[(4-methylcyclohexyl)methyl]sulfonyl}amino)carbonyl]azetidin-1-yl}nicotinate;
ethyl 5-cyano-6-[3-({[(3-cyanophenyl)sulfonyl]amino}carbonyl)azetidin-1-yl]-2-(difluoromethyl)nicotinate;
ethyl 5-cyano-6-[3-({[(4-cyanophenyl)sulfonyl]amino}carbonyl)azetidin-1-yl]-2-(difluoromethyl)nicotinate;
ethyl 5-cyano-2-(difluoromethyl)-6-{3-[({[4-(trifluoromethoxy)phenyl]sulfonyl}amino)carbonyl]azetidin-1-yl}nicotinate;
ethyl 5-cyano-2-(difluoromethyl)-6-{3-[({[2-(trifluoromethoxy)phenyl]sulfonyl}amino)carbonyl]azetidin-1-yl}nicotinate;
ethyl 5-cyano-6-[3-({[(2-cyanobenzyl)sulfonyl]amino}carbonyl)azetidin-1-yl]-2-(difluoromethyl)nicotinate;
ethyl 5-cyano-2-(difluoromethyl)-6-(3-{[(2-naphthylsulfonyl)amino]carbonyl}azetidin-1-yl)nicotinate;
ethyl 6-(3-{[(butylsulfonyl)amino]carbonyl}azetidin-1-yl)-5-cyano-2-(difluoromethyl)nicotinate;
ethyl 5-cyano-6-[4-({[(3-cyanophenyl)sulfonyl]amino}carbonyl)piperidin-1-yl]-2-(difluoromethyl)nicotinate;
ethyl 5-cyano-6-[4-({[(4-cyanophenyl)sulfonyl]amino}carbonyl)piperidin-1-yl]-2-(difluoromethyl)nicotinate;
ethyl 5-cyano-2-(difluoromethyl)-6-{4-[({[4-(trifluoromethoxy)phenyl]sulfonyl}amino)carbonyl]piperidin-1-yl}nicotinate;
ethyl 5-cyano-2-(difluoromethyl)-6-{4-[({[2-(trifluoromethoxy)phenyl]sulfonyl}amino)carbonyl]piperidin-1-yl}nicotinate;
ethyl 5-cyano-6-[4-({[(2-cyanobenzyl)sulfonyl]amino}carbonyl)piperidin-1-yl]-2-(difluoromethyl)nicotinate;
ethyl 5-cyano-2-(difluoromethyl)-6-(4-{[(2-naphthylsulfonyl)amino]carbonyl}piperidin-1-yl)nicotinate;
ethyl 6-(4-{[(butylsulfonyl)amino]carbonyl}piperidin-1-yl)-5-cyano-2-(difluoromethyl)nicotinate;
ethyl 6-(3-{2-[(benzylsulfonyl)amino]-2-oxoethyl}pyrrolidin-1-yl)-5-cyano-2-(trifluoromethyl)nicotinate;
ethyl 5-cyano-6-[3-(2-oxo-2-{[(2-phenylethyl)sulfonyl]amino}ethyl)pyrrolidin-1-yl]-2-(trifluoromethyl)nicotinate;
ethyl 6-[3-(2-{[(5-chloro-2-thienyl)sulfonyl]amino}-2-oxoethyl)pyrrolidin-1-yl]-5-cyano-2-(trifluoromethyl)nicotinate;
ethyl 5-cyano-6-[3-({[(4-fluorobenzyl)sulfonyl]amino}carbonyl)azetidin-1-yl]-2-(trifluoromethyl)nicotinate;
ethyl 5-cyano-6-[3-({[(3-fluorobenzyl)sulfonyl]amino}carbonyl)azetidin-1-yl]-2-(trifluoromethyl)nicotinate;
ethyl 5-cyano-6-[3-({[(2-fluorobenzyl)sulfonyl]amino}carbonyl)azetidin-1-yl]-2-(trifluoromethyl)nicotinate;
ethyl 5-cyano-6-[3-({[(4-methylbenzyl)sulfonyl]amino}carbonyl)azetidin-1-yl]-2-(trifluoromethyl)nicotinate;
ethyl 5-cyano-6-[3-({[(3-methylbenzyl)sulfonyl]amino}carbonyl)azetidin-1-yl]-2-(trifluoromethyl)nicotinate;
ethyl 6-[3-({[(4-chlorobenzyl)sulfonyl]amino}carbonyl)azetidin-1-yl]-5-cyano-2-(trifluoromethyl)nicotinate;
ethyl 6-[3-({[(2-chlorobenzyl)sulfonyl]amino}carbonyl)azetidin-1-yl]-5-cyano-2-(trifluoromethyl)nicotinate;
ethyl 6-[3-({[(3-chlorobenzyl)sulfonyl]amino}carbonyl)azetidin-1-yl]-5-cyano-2-(trifluoromethyl)nicotinate;
ethyl 5-cyano-6-[3-({[(2,4-dichlorobenzyl)sulfonyl]amino}carbonyl)azetidin-1-yl]-2-(trifluoromethyl)nicotinate;
ethyl 6-[3-({[(5-chloro-2-thienyl)sulfonyl]amino}carbonyl)azetidin-1-yl]-5-cyano-2-(trifluoromethyl)nicotinate;
ethyl 5-cyano-6-[4-({[(4-fluorobenzyl)sulfonyl]amino}carbonyl)piperidin-1-yl]-2-(trifluoromethyl)nicotinate;
ethyl 5-cyano-6-[4-({[(3-fluorobenzyl)sulfonyl]amino}carbonyl)piperidin-1-yl]-2-(trifluoromethyl)nicotinate;
ethyl 5-cyano-6-[4-({[(2-fluorobenzyl)sulfonyl]amino}carbonyl)piperidin-1-yl]-2-(trifluoromethyl)nicotinate;
ethyl 5-cyano-6-[4-({[(4-methylbenzyl)sulfonyl]amino}carbonyl)piperidin-1-yl]-2-(trifluoromethyl)nicotinate;
ethyl 5-cyano-6-[4-({[(3-methylbenzyl)sulfonyl]amino}carbonyl)piperidin-1-yl]-2-(trifluoromethyl)nicotinate;
ethyl 6-[4-({[(4-chlorobenzyl)sulfonyl]amino}carbonyl)piperidin-1-yl]-5-cyano-2-(trifluoromethyl)nicotinate;
ethyl 6-[4-({[(2-chlorobenzyl)sulfonyl]amino}carbonyl)piperidin-1-yl]-5-cyano-2-(trifluoromethyl)nicotinate;
ethyl 6-[4-({[(3-chlorobenzyl)sulfonyl]amino}carbonyl)piperidin-1-yl]-5-cyano-2-(trifluoromethyl)nicotinate;
ethyl 5-cyano-6-[4-({[(2,4-dichlorobenzyl)sulfonyl]amino}carbonyl)piperidin-1-yl]-2-(trifluoromethyl)nicotinate;
ethyl 6-[4-({[(5-chloro-2-thienyl)sulfonyl]amino}carbonyl)piperidin-1-yl]-5-cyano-2-(trifluoromethyl)nicotinate;
ethyl 5-cyano-6-[3-({[(2-fluorobenzyl)sulfonyl]amino}carbonyl)azetidin-1-yl]-2-(fluoromethyl)nicotinate;
ethyl 5-cyano-6-[3-({[(3-fluorobenzyl)sulfonyl]amino}carbonyl)azetidin-1-yl]-2-(fluoromethyl)nicotinate;
ethyl 5-cyano-6-[3-({[(4-fluorobenzyl)sulfonyl]amino}carbonyl)azetidin-1-yl]-2-(fluoromethyl)nicotinate;
ethyl 6-[3-({[(2-chlorobenzyl)sulfonyl]amino}carbonyl)azetidin-1-yl]-5-cyano-2-(fluoromethyl)nicotinate;
ethyl 6-[3-({[(3-chlorobenzyl)sulfonyl]amino}carbonyl)azetidin-1-yl]-5-cyano-2-(fluoromethyl)nicotinate;
ethyl 6-[3-({[(4-chlorobenzyl)sulfonyl]amino}carbonyl)azetidin-1-yl]-5-cyano-2-(fluoromethyl)nicotinate;
ethyl 5-cyano-2-(fluoromethyl)-6-[3-({[(3-methylbenzyl)sulfonyl]amino}carbonyl)azetidin-1-yl]nicotinate;
ethyl 5-cyano-2-(fluoromethyl)-6-[3-({[(4-methylbenzyl)sulfonyl]amino}carbonyl)azetidin-1-yl]nicotinate;
ethyl 5-cyano-6-[3-({[(2,4-dichlorobenzyl)sulfonyl]amino}carbonyl)azetidin-1-yl]-2-(fluoromethyl)nicotinate;
ethyl 5-cyano-2-(fluoromethyl)-6-{3-[({[(4-methylcyclohexyl)methyl]sulfonyl}amino)carbonyl]azetidin-1-yl}nicotinate;
ethyl 5-cyano-6-[4-({[(2-fluorobenzyl)sulfonyl]amino}carbonyl)piperidin-1-yl]-2-(fluoromethyl)nicotinate;
ethyl 5-cyano-6-[4-({[(3-fluorobenzyl)sulfonyl]amino}carbonyl)piperidin-1-yl]-2-(fluoromethyl)nicotinate;
ethyl 5-cyano-6-[4-({[(4-fluorobenzyl)sulfonyl]amino}carbonyl)piperidin-1-yl]-2-(fluoromethyl)nicotinate;
ethyl 6-[4-({[(2-chlorobenzyl)sulfonyl]amino}carbonyl)piperidin-1-yl]-5-cyano-2-(fluoromethyl)nicotinate;
ethyl 6-[4-({[(3-chlorobenzyl)sulfonyl]amino}carbonyl)piperidin-1-yl]-5-cyano-2-(fluoromethyl)nicotinate;
ethyl 6-[4-({[(4-chlorobenzyl)sulfonyl]amino}carbonyl)piperidin-1-yl]-5-cyano-2-(fluoromethyl)nicotinate;
ethyl 5-cyano-2-(fluoromethyl)-6-[4-({[(3-methylbenzyl)sulfonyl]amino}carbonyl)piperidin-1-yl]nicotinate;
ethyl 5-cyano-2-(fluoromethyl)-6-[4-({[(4-methylbenzyl)sulfonyl]amino}carbonyl)piperidin-1-yl]nicotinate;
ethyl 5-cyano-6-[4-({[(2,4-dichlorobenzyl)sulfonyl]amino}carbonyl)piperidin-1-yl]-2-(fluoromethyl)nicotinate;
ethyl 5-cyano-2-(fluoromethyl)-6-{4-[({[(4-methylcyclohexyl)methyl]sulfonyl}amino)carbonyl]piperidin-1-yl}nicotinate;
ethyl 6-(3-{2-[(benzylsulfonyl)amino]-2-oxoethyl}azetidin-1-yl)-5-cyano-2-(difluoromethyl)nicotinate;
ethyl 5-cyano-6-(3-{[(2-cyanobenzyl)sulfonyl]carbamoyl}azetidin-1-yl)-2-(trifluoromethyl)nicotinate;
ethyl 5-cyano-6-(3-{[(2,6-difluorobenzyl)sulfonyl]carbamoyl}azetidin-1-yl)-2-(fluoromethyl)nicotinate;
ethyl 5-cyano-2-(fluoromethyl)-6-(3-{[(4-fluoro-3-methylbenzyl)sulfonyl]carbamoyl}azetidin-1-yl)nicotinate;
ethyl 6-(3-{[(2-chloro-4-fluorobenzyl)sulfonyl]carbamoyl}azetidin-1-yl)-5-cyano-2-(fluoromethyl)nicotinate;
ethyl 5-cyano-2-(fluoromethyl)-6-(3-{[(2,3,6-trifluorobenzyl)sulfonyl]carbamoyl}azetidin-1-yl)nicotinate;
ethyl 5-cyano-6-(3-{[(2,4-difluorobenzyl)sulfonyl]carbamoyl}azetidin-1-yl)-2-(fluoromethyl)nicotinate;
ethyl 6-(3-{[(4-chloro-2-fluorobenzyl)sulfonyl]carbamoyl}azetidin-1-yl)-5-cyano-2-(fluoromethyl)nicotinate;
ethyl 5-cyano-6-(3-{[(2,6-difluorobenzyl)sulfonyl]carbamoyl}azetidin-1-yl)-2-(difluoromethyl)nicotinate;
ethyl 5-cyano-2-(difluoromethyl)-6-(3-{[(4-fluoro-3-methylbenzyl)sulfonyl]carbamoyl}azetidin-1-yl)nicotinate;
ethyl 6-(3-{[(2-chloro-4-fluorobenzyl)sulfonyl]carbamoyl}azetidin-1-yl)-5-cyano-2-(difluoromethyl)nicotinate;
ethyl 5-cyano-2-(difluoromethyl)-6-(3-{[(5-fluoro-2-methylbenzyl)sulfonyl]carbamoyl}azetidin-1-yl)nicotinate;
ethyl 5-cyano-6-(3-{[(2,4-difluorobenzyl)sulfonyl]carbamoyl}azetidin-1-yl)-2-(difluoromethyl)nicotinate;
ethyl 6-(3-{[(4-chloro-2-fluorobenzyl)sulfonyl]carbamoyl}azetidin-1-yl)-5-cyano-2-(difluoromethyl)nicotinate;
ethyl 5-cyano-6-(3-{[(2,6-difluorobenzyl)sulfonyl]carbamoyl}azetidin-1-yl)-2-(trifluoromethyl)nicotinate;
ethyl 5-cyano-6-(3-{[(4-fluoro-3-methylbenzyl)sulfonyl]carbamoyl}azetidin-1-yl)-2-(trifluoromethyl)nicotinate;
ethyl 6-(3-{[(2-chloro-4-fluorobenzyl)sulfonyl]carbamoyl}azetidin-1-yl)-5-cyano-2-(trifluoromethyl)nicotinate;
ethyl 5-cyano-6-(3-{[(5-fluoro-2-methylbenzyl)sulfonyl]carbamoyl}azetidin-1-yl)-2-(trifluoromethyl)nicotinate;
ethyl 5-cyano-6-(3-{[(2,3,6-trifluorobenzyl)sulfonyl]carbamoyl}azetidin-1-yl)-2-(trifluoromethyl)nicotinate;
ethyl 6-(3-{[(4-chloro-2-fluorobenzyl)sulfonyl]carbamoyl}azetidin-1-yl)-5-cyano-2-(trifluoromethyl)nicotinate;
ethyl 5-cyano-6-(4-{[(2,6-difluorobenzyl)sulfonyl]carbamoyl}piperidin-1-yl)-2-(difluoromethyl)nicotinate;
ethyl 5-cyano-2-(difluoromethyl)-6-(4-{[(4-fluoro-3-methylbenzyl)sulfonyl]carbamoyl}piperidin-1-yl)nicotinate;
ethyl 5-cyano-2-(fluoromethyl)-6-(3-{[(2-fluoro-5-methylbenzyl)sulfonyl]carbamoyl}azetidin-1-yl)nicotinate;
ethyl 5-cyano-6-(4-{[(2-fluoro-5-methylbenzyl)sulfonyl]carbamoyl}piperidin-1-yl)-2-(trifluoromethyl)nicotinate;
ethyl 5-cyano-6-(3-{[(2-fluoro-5-methylbenzyl)sulfonyl]carbamoyl}azetidin-1-yl)-2-(trifluoromethyl)nicotinate;
ethyl 5-cyano-2-(difluoromethyl)-6-(4-{[(2-fluoro-5-methylbenzyl)sulfonyl]carbamoyl}piperidin-1-yl)nicotinate;
ethyl 5-cyano-2-(difluoromethyl)-6-(3-{[(3-methoxybenzyl)sulfonyl]carbamoyl}azetidin-1-yl)nicotinate;
ethyl 6-{4-[(benzylsulfonyl)carbamoyl]piperidin-1-yl}-5-cyano-2-(pentafluoroethyl)nicotinate;
ethyl 6-{3-[(benzylsulfonyl)carbamoyl]azetidin-1-yl}-5-cyano-2-(pentafluoroethyl)nicotinate;
ethyl 6-{3-[(benzylsulfonyl)carbamoyl]azetidin-1-yl}-5-cyano-2-(1-fluoroethyl)nicotinate;
ethyl 6-{4-[(benzylsulfonyl)carbamoyl]piperidin-1-yl}-5-cyano-2-(1-fluoroethyl)nicotinate;
ethyl 6-(4-{[(2-chloro-4-fluorobenzyl)sulfonyl]carbamoyl}piperidin-1-yl)-5-cyano-2-(fluoromethyl)nicotinate;
ethyl 5-cyano-6-(4-{[(2,4-difluorobenzyl)sulfonyl]carbamoyl}piperidin-1-yl)-2-(fluoromethyl)nicotinate;
ethyl 6-{4-[(benzylsulfonyl)carbamoyl]piperidin-1-yl}-2-(chloromethyl)-5-cyanonicotinate;
ethyl 5-cyano-2-(difluoromethyl)-6-(3-{[(2-fluoro-5-methylbenzyl)sulfonyl]carbamoyl}azetidin-1-yl)nicotinate;
ethyl 6-{3-[(benzylsulfonyl)carbamoyl]azetidin-1-yl}-2-(chloromethyl)-5-cyanonicotinate;
ethyl 5-cyano-6-(3-{[(3,4-difluorobenzyl)sulfonyl]carbamoyl}azetidin-1-yl)-2-(difluoromethyl)nicotinate;
ethyl 5-cyano-6-(4-{[(3,4-difluorobenzyl)sulfonyl]carbamoyl}piperidin-1-yl)-2-(difluoromethyl)nicotinate;
ethyl 5-cyano-6-(4-{[(2,4-difluorobenzyl)sulfonyl]carbamoyl}piperidin-1-yl)-2-(difluoromethyl)nicotinate;
ethyl 6-{4-[(benzylsulfonyl)carbamoyl]piperidin-1-yl}-5-cyano-2-(2-fluoroethoxy)nicotinate;
ethyl 6-{3-[(benzylsulfonyl)carbamoyl]azetidin-1-yl}-5-cyano-2-[(2,2,2-trifluoroethoxy)methyl]nicotinate;
ethyl 6-{4-[(benzylsulfonyl)carbamoyl]piperidin-1-yl}-5-cyano-2-[(2,2,2-trifluoroethoxy)methyl]nicotinate;
ethyl 6-{4-[(benzylsulfonyl)carbamoyl]piperidin-1-yl}-5-cyano-2-(difluoromethoxy)nicotinate;
ethyl 6-{4-[(benzylsulfonyl)carbamoyl]piperidin-1-yl}-5-cyano-2-(2,2-difluoroethoxy)nicotinate;
ethyl 6-{4-[(benzylsulfonyl)carbamoyl]piperidin-1-yl}-5-cyano-2-(2,2,2-trifluoroethoxy)nicotinate;
ethyl 5-cyano-2-(difluoromethyl)-6-[3-({[4-(hydroxymethyl)benzyl]sulfonyl}carbamoyl)azetidin-1-yl]nicotinate;
ethyl 5-cyano-2-(difluoromethyl)-6-[4-({[4-(hydroxymethyl)benzyl]sulfonyl}carbamoyl)piperidin-1-yl]nicotinate;
ethyl 6-{3-[(benzylsulfonyl)carbamoyl]azetidin-1-yl}-5-cyano-2-(2,2-difluoroethoxy)nicotinate;
ethyl 5-cyano-2-(2,2-difluoroethoxy)-6-(3-{[(4-fluorobenzyl)sulfonyl]carbamoyl}azetidin-1-yl)nicotinate;
ethyl 5-cyano-2-(2,2-difluoroethoxy)-6-(3-{[(2-fluorobenzyl)sulfonyl]carbamoyl}azetidin-1-yl)nicotinate;
ethyl 5-cyano-6-(3-{[(2,4-difluorobenzyl)sulfonyl]carbamoyl}azetidin-1-yl)-2-(2,2-difluoroethoxy)nicotinate;
isopropyl 6-{3-[(benzylsulfonyl)carbamoyl]azetidin-1-yl}-5-cyano-2-(difluoromethyl)nicotinate;
ethyl 5-cyano-6-[3-({[(4-methylcyclohexyl)methyl]sulfonyl}carbamoyl)azetidin-1-yl]-2-(trifluoromethyl)nicotinate;
or a pharmaceutically acceptable salt thereof.

19. A pharmaceutical composition comprising a compound according to claim 1 and a pharmaceutically acceptable adjuvant, diluent and/or carrier.

20. A method of treatment of a platelet aggregation disorder comprising administering to a patient suffering from such a disorder a therapeutically effective amount of a compound according to claim 1.

21. A method of inhibition of the P2Y₁₂ receptor in a cell comprising administering to a patient having such receptor an effective amount of a compound according to claim 1.
